(19) United States
(12) Patent Application Publication
(10) Pub. No.: US 2024/0148835 A1
(43) Pub. Date: May 9, 2024

(54) TREATING DISORDERS ASSOCIATED WITH DYRK1A DYSFUNCTION

(71) Applicant: UNIVERSITY OF FLORIDA RESEARCH FOUNDATION, INCORPORATED, GAINESVILLE, FL (US)

(72) Inventors: Damon PAGE, Jupiter, FL (US); Jenna LEVY, Jupiter, FL (US)

(21) Appl. No.: 18/274,980

(22) PCT Filed: Jan. 28, 2022
(86) PCT No.: PCT/US2022/014412

§ 371 Date: Jul. 28, 2023

Related U.S. Application Data
(60) Provisional application No. 63/142,720, filed on Jan. 28, 2021.

(51) Int. Cl. A61K 38/30; A61P 25/00; C12N 15/113

(52) U.S. Cl. CPC A61K 38/30; A61P 25/00; C12N 15/1137; C12N 2310/11; C12N 2310/14

(57) ABSTRACT

The present invention provides methods for treating a condition or disorder that is associated with or mediated by dysregulation or dysfunction of the dual-specificity tyrosine phosphorylation-regulated kinase 1A (DYRK1A), e.g., microcephaly, autism spectrum disorder (ASD), or intellectual disability (ID). The methods of the invention involve administering to a subject in need of treatment a pharmaceutical composition that stimulates or upregulates a signaling cascade selected from BDNF-TrkB, mTOR, IGF-1 and ERK/MAPK signaling pathways.

### CROSS REFERENCE TO RELATED APPLICATION

This application claims the benefit of U.S. 63/142,720 filed Jan. 28, 2021, which is incorporated by reference in its entirety for all purposes.

### REFERENCE TO A SEQUENCE LISTING

The Sequence Listing written in file 573965SEQLIST.txt is 159,437 bytes, was created on Jan. 28, 2022, and is hereby incorporated by reference.

### BACKGROUND OF THE INVENTION

The dual-specificity tyrosine phosphorylation-regulated kinase 1A gene (Dyrk1a) encodes a serine/threonine kinase, DYRK1A (7). DYRK1A targets proteins critical for proliferation and differentiation in the developing mammalian brain, including Gsk3β (8), CyclinD1 (9), Foxo1 (10), Notch Intracellular Domain (NICD) (11), and P53 (12). Mutations in Dyrk1a impact cell cycle and proliferation through these phosphorylation targets. Germline Dyrk1a^{+/-} mice exhibit decreased brain and body mass in adulthood as well as behavioral deficits (13-15) and altered proliferation (9, 12, 13). Dyrk1a mutations also cause decreased complexity of cultured neurons (16). However, the molecular mechanisms governing these phenotypes are currently unknown.

A number of conditions or disorders are associated with dysregulation of DYRK1A. For example, loss of function mutations in DYRK1A (OMIM #600855) are strongly associated with autism spectrum disorder (ASD), intellectual disability (ID) and microcephaly in human. Multiple signaling cascades have been implicated in the pathogenesis of ASD, including Wnt/GSK3β, mTOR, and ERK/MAPK signaling (17-21). These molecular cascades are critical for regulating growth and proliferation in the developing mammalian brain (22-24) and have been proposed as signaling “hubs” for various etiologies of ASD (21, 25, 26). Convergence between Wnt/GSK3β, ERK/MAPK and PI3K/Akt/mTOR pathways in the developing brain (27-29) illustrate the abundant crosstalk between signaling cascades implicated in ASD pathobiology. Multiple studies indicate that Dyrk1a may interact with these ASD-associated signaling cascades. For example, Dyrk1a overexpression causes increased MAPK signaling in PC12 cells (30) and increased ERK/Akt activation in mice (31). Dyrk1a is a priming kinase for GSK3β (32), and PI3K/Akt/mTOR signaling is hyperactivated in human brain tissue from patients with Down syndrome, caused by trisomy of chromosome 21 including DYRK1A (33).

There is a unmet need in the art for novel and effective methods for treating the various diseases or conditions that are associated with or mediated by disruption of the normal DYRK1A function. The present invention addresses this and other unmet needs in the art.

### SUMMARY OF THE INVENTION

In one aspect, the invention provides a method of treating a DYRK1A-related disorder comprising administering IGF-1, (1-3)IGF-1, Trofinetide, or NNZ-2591. In some methods, the DYRK1A-related disorder comprises a DYRK1A syndrome, an autism spectrum disorder (ASD), intellectual disability (ID), microcephaly and sociability deficits. In some methods, treating the DYRK1A-related disorder comprises improving sociability, decreasing microcephaly, increasing spine density, and/or improving synaptic function.

In another aspect, the invention provides a method for increasing cortical mass, cortical cell growth, neuronal cell growth, protein synthesis, and/or spinal density in a subject having a loss of function DYRK1A mutation comprising administering IGF-1, (1-3)IGF-1, Trofinetide, or NNZ-2591. In some methods, the increase in cortical cell growth is an increase in cell size and/or cell number. In some methods, the increase in neuronal cell growth is an increase in cell size and/or cell number.

In another aspect, the invention provides a method of treating a DYRK1A-related disorder comprising administering a Gsk3β inhibitor. In some methods, the DYRK1A-related disorder comprises a DRK1A syndrome, an autism spectrum disorder (ASD), intellectual disability (ID), microcephaly and sociability deficits. In some methods, treating the DYRK1A-related disorder comprises improving sociability, decreasing microcephaly, increasing spine density, and/or improving synaptic function.

In some methods, the Gsk30 inhibitor comprises an antisense oligonucleotide or an siRNA wherein the antisense oligonucleotide or the siRNA targets the Gsk3β gene and causes a decrease in expression of the Gsk3β gene. In some methods, the Gsk3β inhibitor comprises SB216763. SAR502250, indirubin-3-oxime, CHIR-99021, AT7519, TWS119, indirubin, SB415286, CHIR-98014, lithium, zinc, tungstate, an indirubin, 6-BIO, hymenialdisine, dibromocantharelline, a meridianin, an aminopyrimidine, CT98014, CT98023, CT99021, TWS119, an arylindolemaleimide, SB-41528, a thiazole, AR-A014418, AZD-1080, a paullone, kenpaullone, alsterpaullone, cazpaullone, an aloisine, a manzamine, manzamine A, a furanosesquiterpene, palinurine, tricantine, a thiadiazolidindione, TDZD-8, NP00111, NP031115, NP031112 (tideglusib), a halomethylketone, HMK-32, or L803-mts.

In another aspect, the invention provides a method for increasing cortical mass, cortical cell growth, neuronal cell growth, protein synthesis, and/or spinal density in a subject having a loss of function DYRK1A mutation comprising administering a Gsk3β inhibitor. In some methods, the increase in cortical cell growth is an increase in cell size and/or cell number. In some methods, the increase in neuronal cell growth is an increase in cell size and/or cell number.

In another aspect, the invention provides a method of treating a DYRK1A-related disorder comprising administering BDNF, TrkB, mTOR, IGF-1 ERK1, or ERK2, nucleic acid encoding BDNF, TrkB, mTOR, IGF-1 ERK1, or ERK2, or a positive effector of BDNF, TrkB, mTOR, IGF-1 ERK1, or ERK2. In some methods, the DYRK1A-related disorder comprises a DYRK1A syndrome, an autism spectrum disorder (ASD), intellectual disability (ID), microcephaly and sociability deficits. In some methods, treating the DYRK1A-related disorder comprises improving sociability, decreasing microcephaly, increasing spine density, and/or improving synaptic function.

In some methods, the positive effector of BDNF comprises 7,8-dihydroxyflavone. In some methods, the positive effector of TrkB comprises 7,8-dihydroxyflavone, deoxygedunin, LM22A-4, HIOC, R7, R13, AS86, Ab1A01, Ab104, Ab4B19, Ab2C03, Ab303, Ab1104, or Ab2908. In some methods, the nucleic acid encoding BDNF, TrkB, mTOR, IGF-1 ERK1, or ERK2 comprises a plasmid, a viral vector, or an mRNA.

In another aspect, the invention provides a method of treating a DYRK1A-related disorder comprising administering a Pten inhibitor. In some methods, the DYRK1A-related disorder comprises a DYRK1A syndrome, an autism spectrum disorder (ASD), intellectual disability (ID), microcephaly and sociability deficits. In some methods, treating the DYRK1A-related disorder comprises improving sociability, decreasing microcephaly, increasing spine density, and/or improving synaptic function.

In some methods, the Pten inhibitor comprises an antisense oligonucleotide or an siRNA wherein the antisense oligonucleotide or the siRNA targets the PTen gene and causes a decrease in expression of the Pten gene. In some methods, the Pten inhibitor comprises bpV(phen), bpV(pic), bpV(HOpic), bpV(pis), VO-OHpic, or SF1670. A further understanding of the nature and advantages of the present invention may be realized by reference to the remaining portions of the specification and claims.

### DESCRIPTION OF THE DRAWINGS

FIG. 1. Cortical mutations in Dyrk1a model microcephaly and ASD-relevant behaviors. (A) Breeding scheme used to generate cHets and cKOs. cKOs do not survive past birth and are analyzed only at P0 as reflected in the data points. (B-C) Cortical deletion of Dyrk1a causes decreased brain (B) and cortex (C) mass at birth, throughout development, and in adulthood. P0 analyzed by one-way ANOVA with Tukey's post hoc multiple comparisons tests showed significant effects of genotype on brain (F_{2,29}=41.15, P<0.0001) and cortex mass (F_{2, 21}=22.14, P<0.0001). P7 and adult timepoints were analyzed by independent sample t-test (P7 brain: t₂₈=4.472, P=0.00186; P7 cortex: t₁₇=2.437, P=0.026081; adult brain: t₂₅=3.602, P=0.001366 adult cortex: t₁₁=3.811, P=0.002889). N=8-15/genotype per timepoint. (D) Dorsal view of dissected whole brains at birth from each genotype (left panel). Representative images of coronal brain sections showcasing the morphological abnormalities in the cKO brain at birth (top panel). Representative coronal brain images of adult control cHet brains at Bregma 0.26 mm (bottom right panel) with schematic showing measurements used in (G-I) to determine cortical thickness (ML=midline). (E) The ventricles in adult cHet brains are significantly larger compared to controls (t₅=4.551, P=0.0061). (F) The corpus callosum is significantly thinner in adult cHets (t₄=2.833, P=0.0472). Ventricle and corpus callosum measurements were analyzed by independent sample t-tests. N=3-4/genotype. (G) The thickness of the cortex in the cKO brain is significantly reduced at multiple measurement points with no gross alterations in the cHets at birth. Analysis by repeated measures two-way ANOVA (F_{10,36}=1.244, P=0.2977) shows no significant interaction of angle measured X genotype. Despite absence of significant interactions between layer and genotype, planned comparisons between genotypes at each layer (with Tukey's post hoc multiple comparisons tests) showed significant differences (one-way ANOVAs; 120°: F_{2, 6}=1.893, P=0.2305; 90°: F_{2, 6}=4.871, P=0.0554; 60°: F_{2, 6}=7.852, P=0.0211; 45°: F_{2, 6}=19.99, P=0.0022; 30°: F_{2, 6}=38.20, P=0.0004; ML°: F_{2,6}=13.89, P=0.0056). (H) At P7, the cortex is thinner at multiple angles in cHets. Repeated measure two-way ANOVA with Sidak's post hoc multiple comparisons tests showed a significant interaction between genotype and measurement angle (F_{5,20}=12.52, P<0.0001). (I) Adult cHet cortex is thinner at multiple measurement angles as analyzed by repeated measures two-way ANOVA (F_{5,30}=0.8433, P=0.5299). While there were no significant interactions between layer and genotype, planned comparisons between genotypes at each layer showed significant differences (independent sample t-tests; 120°: t₆=2.14646, P=0.075474; 90°: t₆=2.67184, P=0.036936; 60°: t₆=2.34475, P=0.057470; 45°: t₆=3.75555, P=0.009448; 30°: t₆=2.74530, P=0.033499; ML°: t₆=2.96656, P=0.025069). N=3-4/genotype. Results from post hoc and t-tests indicated on graphs. Error bars represent mean+/−SEM. *P<0.05, **P<0.01, ***P<0.001, ****P<0.0001.

FIG. 2: cHets exhibit autism-relevant behaviors but no alterations in locomotor activity, anxiety-like behavior, or depressive-like behavior. (A) Analysis of 3-chamber social approach assay shows that controls exhibit a significant preference for the chamber with the stimulus mouse compared to the chamber with the empty tube (paired sample t-test within control group, t₁₀=3.576, P=0.0050). cHets do not exhibit this preference and spend equal time in the social and nonsocial chambers (paired sample t-test within cHet group, t₁₃=0.5569, P=0.5871). (B) cHets spend significantly less time with the social stimulus compared to controls. Analyzed by independent sample t-test (t₂₃=2.082, P=0.0487). (C) Averaged heat map from controls and cHets during 3-chamber social approach assay where “M” represents the chamber with the stimulus mouse and “E” represents the chamber with the empty tube. N=11-14 males/genotype. (D) In the pre-pulse inhibition assay, there is no difference in percent inhibition between controls and cHets. Repeated measures two-way ANOVA shows no significant interaction between genotype and volume of pre-pulse (F_{2,52}=0.9034, P=0.4114); however, there is a significant effect of pre-pulse volume (F_{2,52}=0.70.39, P<0.0001). (E) Acoustic startle measured within the pre-pulse inhibition assay shows no differences between controls and cHets. While there is no significant interaction between timing and genotype shown by repeated measures two-way ANOVA, (F_{2,52}=0.6061, P=0.5493), there is a trend towards an effect of genotype (F_{1,26}=3.638, P=0.0676) and significant effect of timing of startle (F_{1.408,36.62}=4.339, P=0.0319). (F) cHets bury significantly fewer marbles in 30 minutes compared to controls (t₂₆=3.806, P=0.0008). (G-I) In the open field test, cHets do not exhibit any alterations in distance traveled (G; t₂₅=1.705, P=0.1005), velocity (H; t₂₅=1.698, P=0.1018), or thigmotaxis (I; Center; t₂₅=0.498910, P=0.622205; Sides/corners; t₂₅=0.511655, P=0.613383). (J-K) In the elevated plus maze, cHets do not exhibit alterations in closed arm duration (J; t₂₄=0.04611, P=0.9636) or open arm duration (K; t₂₄=1.283, P=0.2117). (L) In the tail suspension test, cHets do not exhibit any alterations in immobility (t₂₃=0.9501, P=0.3519). For all behavioral experiments, N=11-14 males/genotype. Analyzed by independent sample t-test unless otherwise specified. Results from post hoc and t-tests indicated on graphs. Error bars represent mean+/−SEM. *P<0.05, **P<0.01, ***P<0.001, ****P<0.0001.

FIG. 3: Microcephaly in conditional Dyrk1a mutants is driven by decreased cell size at birth and decreased cell number after birth. (A) Schematic of isotropic fractionator protocol. (B) At birth, P0 cHets and cKOs do not exhibit alterations in cell number in the cortex. At P7 and adulthood, cell number is significantly reduced relative to controls. P0 analyzed by one-way ANOVA with Tukey's post hoc multiple comparisons tests (shown on graph; F_{2,16}=0.01390, P=0.9862). P7 and adult timepoints analyzed by independent sample t-test (P7: t₈=2.663, P=0.028690; adult: t₈=2.579, P=0.032651). (C) At P0, cortical cell density is significantly increased in cKOs and is normalized by P7 and through adulthood. P0 analyzed by one-way ANOVA with Tukey's post hoc multiple comparisons tests (shown on graph; F_{2,16}=18.31, P<0.0001). P7 and adult timepoints analyzed by independent sample t-tests (P7: t₈=1.345, P=0.215528; adult: t₈=1.942, P=0.0881). For all ages, N=4-8/genotype. (D) Representative images of Nissl-stained cell somas in layer V of somatosensory cortex at birth with quantification showing significant decreases in multiple layers of P0 cHet and cKO cortices. Analyzed by one-way ANOVA [I-IV (F_{2,6}=18.83, P=0.0026), V (F_{2,6}=65.83, P<0.0001), VI (F_{2,6}=21.62 P=0.0018)] with Tukey's post hoc multiple comparisons tests. (E) Representative Nissl-stained cell somas at P7 with quantification showing a significant decrease in layer V of somatosensory cortex in cHets [independent sample t-tests: II/III (t₆=1.842, P=0.114989), IV (t₆=1.897, P=0.106656), V (t₆=2.462, P=0.048976), VI (t₆=1.328, P=0.23571)]. (F) Representative images of Nissl-stained cell somas in layer V of somatosensory cortex in adult animals with quantification showing that somas in layers V and VI are significantly smaller in cHets [independent sample t-tests (t₄=1.612, P=0.182235), IV (t₄=1.045, P=0.355062), V (t₄=5.107, P=0.006961), VI (t₄=7.862, P=0.001414)]. For all ages, N=3-4/genotype. (G) Representative images of CC3 staining in the cortex at birth. (H) Representative images of increased CC3 in the cortex at P4 with quantification of CC3+ cells per cortical hemisphere using immunohistochemistry. Analyzed by independent sample t-test (t₄=10.21, P=0.0005). N=3/genotype. (I) Breeding scheme used to generate Rbp4-cre⁺; Dyrk1a^{loxP/loxP} pups. (J) Representative images of Nissl-stained cell somas in layer V of somatosensory cortex at birth in Rbp4-cKOs. (K) Quantification of soma size using Nissl-stained sections shows that Rbp4-cKOs exhibit decreased soma size in layer V of somatosensory cortex compared to controls [independent sample t-tests: II-IV (t₄=4.00343, P=0.091760), V (t₄=6.48343, P=0.006136), VI (t₄=2.46921, P=0.021545)]. N=3/genotype. (L) Representative images of CC3 staining in the cortex at birth shows that Rbp4-cKOs exhibit increased CC3+ apoptotic cells in layer V. Results from post hoc and t-tests indicated on graphs. Error bars represent mean+/−SEM. *P<0.05, **P<0.01, ***P<0.001, ****P<0.0001.

FIG. 4: Dyrk1a cHets exhibit altered neuronal morphology of layer V pyramidal neurons and decreased cortico-subcortical projection density. (A) Representative Golgi-stained layer V area of the mPFC from adult control and cHet brains. (B) Reconstructed neurons from the representative images in (A) using Neuromantic software. (C) Sholl analysis of neuronal arborization on individual neurons shows that cHets exhibit significantly decreased neuronal arborization (independent sample t-test of area under the curve generated for each genotype: t₄=8.818, P=0.0009). N=10-15 neurons/mouse, 3 mice/genotype across two sections. (D) Adult cHets exhibit decreased soma size in layer V pyramidal neurons in the mPFC (t₄=4.775, P=0.0088). (E) cHet pyramidal neurons exhibit more circular somas measured by the circularity plugin in ImageJ (t₄=2.991, P=0.0403). (F) cHet layer V pyramidal neurons contain less complex basal branches, measured by counting the endpoints on the reconstructed neurons (apical: t₄=0.9301, P=0.404961; basal: t₄=4.026, P=0.015785). (G) Overview of injection site in the mPFC where 50 nL AAV-DIO-EYFP was injected in adult mice (Bregma 1.98 mm). Zoomed inset shows comparable density of virus in the mPFC. Inset of the BLA shows decreased EYFP+ projections in the BLA in cHets (Bregma −1.58 mm). (H) cHets exhibit decreased projection density in the BLA (t₄=2.933, P=0.0427). Measured by fiber reconstruction in ImageJ relative to size of the ROI. N=3/genotype. (I) Control sagittal brain section showing tdTomato expression under the Emx1-cre promoter. Dotted rectangle delineates the area in which the image was taken with enhancement of the red fluorophore expressed in the fibers of the corticospinal tract (CST). When comparing matched sections from a control and cHet, the qualitative difference is striking. (J) Quantification of the fiber density from the cortex to the corticospinal tract shows cHets have decreased intensity of the red fluorophore expressed in these fibers (t₄=3.977, P=0.0164). Analyzed by averaging the mean gray intensity in the CST in 3 matched sections per animal normalized to the size of the CST. N=3/genotype. Results analyzed by independent sample t-tests unless otherwise specified. Results from post hoc and t-tests indicated on graphs. Error bars represent mean+/−SEM. *P<0.05, **P<0.01, ***P<0.001, ****P<0.0001.

FIG. 5: Dyrk1a targets TrkB-BDNF signaling cascades in the developing cortex. (A) Volcano plot showing number of significantly altered proteins after FDR correction. A false discovery rate (FDR) of 0.01 was used and only peptides that had an adjusted P value of <0.05 after correction were analyzed. Additionally, only phosphopeptides with a probability value >75% were included. Individual proteins were used to compare cKOs to controls using the ANOVA option in Proteome Discoverer, and adjusted P values were generated. N=6-7 mice/genotype, males and females. (B) Log₂ abundance ratio of proteins (identified in proteome data set) that are encoded for by ASD/ID risk genes or genes involved in regulating brain size as identified in DBDB. For SFARI gene, only genes with confidence score 1-3 were considered. (C) Volcano plot showing number of significantly altered peptides after FDR correction. N=3-4 female mice/genotype. (D) Log₂ abundance ratio of proteins (identified in phospho-proteome dataset) that are encoded for by ASD/ID risk genes or genes involved in regulating brain size as identified in DBDB. For SFARI gene, only genes with confidence score 1-3 were considered. (E) Dot plot of GO terms enriched in both down-regulated proteins and down-regulated phosphopeptides. Abbreviations: development (dev.), cellular (cell.), morphogenesis (morph.), projection (proj.), membrane (mem.), and regulation (reg.). Dot size corresponds to the number of proteins in the dataset identified under the GO term. Color corresponds to FDR-corrected P value. The size of the dot is relative to the dataset, such that the smallest dot represents 6 proteins. (F) Western blot of P0 control and cKO cortical lysate using antibodies against phospho-proteins and total proteins identified by mass spectrometry. (G) Quantification of western blot where phospho-proteins are normalized to total protein and to a loading control ((3-actin). Analyzed by multiple t-tests. N=3-4/genotype. Results from post hoc and t-tests indicated on graphs. Error bars represent mean+/−SEM. *P<0.05, **P<0.01, ***P<0.001, ****P<0.0001.

FIG. 6: Activation of the mTOR pathway is decreased in layer V and throughout the cortex of conditional Dyrk1a mutants. (A) P0 cKO cortices exhibit decreased activation of mTOR signaling molecules. Western blot of cortical lysate from P0 pups with quantification measured as relative abundance in ImageJ. Phospho-protein was normalized to total protein. Analyzed by one-way ANOVA with Sidak's post hoc multiple comparisons tests (p-mTOR/mTOR: F_{2,12}=3.406, P=0.0674; p-S6K/S6K: F_{2,14}=3.757, P=0.0494; p-S6/S6: F_{2,11}=7.371, P=0.0093; p-ERK1/ERK1: F_{2, 6}=13.55, P=0.006; p-ERK2/ERK2: F_{2, 6}=28.9, P=0.0008). (B) P7 cHets exhibit decreased activation of S6K in the cortex (t₄=2.85833, P=0.046007). Quantified using relative abundance in ImageJ. (C) Adult cHets exhibit decreased activation of S6K in the cortex (t₆=2.49104, P=0.047096). Quantified using relative abundance in ImageJ. For all ages, N=3-5/genotype. (D) Representative images from coronal sections in Si showing decreased p-S6 intensity in P0 cHets and cKOs. (E) p-S6 intensity quantified by measuring mean gray intensity of the green fluorophore (Alexafluor 488) per Ctip2+ layer V neuron. 25-30 neurons measured per section in 2-3 plane-matched sections per animal. Analyzed by one-way ANOVA with Sidak's post hoc multiple comparisons tests (F_{2,6}=88.93 P<0.0001). (F) p-S6 per layer V neuron is decreased at P7 (t₄=3.013, P=0.0394). (G) p-S6 per layer V neuron is decreased in adults (t₆=3.374, P=0.0150). For all ages, N=3/genotype. Results analyzed by independent sample t-tests unless otherwise specified. Results from post hoc and t-tests indicated on graphs. Error bars represent mean+/−SEM. *P<0.05, **P<0.01, ***P<0.001, ****P<0.0001.

FIG. 7: Genetic suppression of Pten and pharmacological treatment with (1-3)IGF-1 rescues microcephaly and cellular undergrowth in neonatal cHets. (A) Schematic of a genetic rescue approach. dHet (Emx1-cre; Dyrk1^{lox/P+}; Pten^{loxP/+}) pups were collected at P7 for analysis. (B) Genetic suppression of Pten in Dyrk1a cHets rescues cortex mass (F_{2,6}=11.76, P=0.0084). N=3/genotype. (C) Representative images of Nissl-stained layer V cell somas (top) and p-S6/Ctip2 stained cells (bottom). (D) Quantification of cell soma size in layer V shows a partial rescue of cell size in dHets (F_{2,6}=6.724, P=0.0294). N=3/genotype. (E) Quantification of p-S6 fluorescence per layer V neuron as identified by positive staining for Ctip2. dHets rescued the decreased p-S6 in cHets (F_{2,6}=17.71, P=0.003). N=3/genotype. (F) Schematic of neonatal (1-3)IGF-1 injection paradigm. Pups were injected with 10 μg/g (1-3)IGF-1 in saline in the morning from P1 daily until P7. They were sacrificed 8 hours post-injection on P7. (G) (1-3)IGF-1 injection rescues the decreased cortex mass observed in cHets treated with vehicle. Two-way ANOVA with Tukey's post hoc multiple comparisons tests showed a significant interaction of drug and genotype (F_{1,13}=13.70, P=0.0027). N=3-7/genotype. (H) Representative images of Nissl-stained layer V cell somas (top) and p-S6/Ctip2 stained cells (bottom). (I) Neonatal (1-3)IGF-1 injection rescues the decreased soma size observed in cHets treated with vehicle. Two-way ANOVA with Tukey's post hoc multiple comparisons tests showed a significant interaction of drug and genotype (F_{1,8}=13.4, P=0.0064). N=3/genotype. (J) Neonatal (1-3)IGF-1 injection rescues the decreased p-S6 in layer V neurons observed in vehicle-treated cHets. Two-way ANOVA with Tukey's post hoc multiple comparisons tests showed a significant interaction of drug and genotype (F_{1,8}=24.19, P=0.0012). N=3/genotype. Results analyzed by one-way ANOVA with Tukey's post hoc multiple comparisons tests unless otherwise specified. Results from post hoc and t-tests indicated on graphs. Error bars represent mean+/−SEM. *P<0.05, **P<0.01, ***P<0.001, ****P<0.0001.

FIG. 8: Expression of Dyrk1a in the mutant cortex over time and analysis of cortical thickness. Western blot of cortical lysate from P0 pups with quantification measured as relative abundance in ImageJ. Dyrk1a was normalized to β-Actin. P0 analyzed by one-way ANOVA with Tukey's post hoc multiple comparisons tests (F_{2,8}=22.01, P=0.0006). P7 and adult measured by independent sample t-tests (P7: t₈=3.099, P=0.014, Adult: t₆=7.782, P=0.0002). N=3-5/genotype. Results from post hoc and t-tests indicated on graphs. Error bars represent mean+/−SEM. *P<0.05, **P<0.01, ***P<0.001, ****P<0.0001.

FIG. 9: Alterations in neuronal and non-neuronal populations drive postnatal microcephaly in cHets. (A) The proportion of Neu-N+ nuclei is unchanged at P0 and adult but decreased at P7. P0 analyzed by one-way ANOVA F_{2,8}=1.704, P=0.2419. P7 and adults measured by independent sample t-tests (P7: t₄=4.866, P=0.008, Adult: t₆=2.323, P=0.84). (B) The proportion of Neu-N− nuclei is unchanged at P0 and adult but increased at P7. P0 analyzed by one-way ANOVA F_{2,8}=1.704, P=0.2419. P7 and adults measured by independent sample t-tests (P7: t₄=4.866, P=0.008, Adult: t₆=2.323, P=0.84). (C) The total number of neuronal nuclei is unchanged at P0 but decreased at P7 and adult. P0 analyzed by one-way ANOVA F_{2,7}=1.162, P=0.3667. P7 and adults measured by independent sample t-tests (P7: t₄=4.718, P=0.009, Adult: t₆=2.577, P=0.042). (D) The total number of non-neuronal nuclei is unchanged at P0 and P7 but decreased in adults. P0 analyzed by one-way ANOVA F_{2,7}=3.148, P=0.1059. P7 and adults measured by independent sample t-tests (P7: t₄=0.37, P=0.73, Adult: t₆=3.408, P=0.014). N=3-4/genotype. Results from post hoc and t-tests indicated on graphs. Error bars represent mean+/−SEM. *P<0.05, **P<0.01, ***P<0.001, ****P<0.0001.

FIG. 10: Conditional Dyrk1a mutants exhibit decreased proportion of layer V neurons. (A) Representative image of the cortical column in somatosensory cortex of P0 mice stained with Ctip2. Quantification shows there are no alterations in Ctip2+ proportions in cHets. In cKOs, there is a significant decrease in bins related to the approximate layer V location and an increase in the uppermost bin closest to the pial surface (PS) and no alterations near the ventricular zone (VZ). Repeated measure two-way ANOVA with Tukey's post hoc multiple comparisons tests show a significant interaction between bin and genotype (F_{18,54}=4.541, P<0.0001). (B) At P7, there is a significant decrease in the Ctip2+ proportion in the bin corresponding to layer V. Repeated measure two-way ANOVA with Sidak's post hoc multiple comparisons tests show a significant interaction between bin and genotype (F_{9,18}=3.264 P=0.0156). (C) In adulthood, there is a significant decrease in the Ctip2+ proportion in the bins corresponding to layer V and VI. Repeated measure two-way ANOVA with Sidak's post hoc multiple comparisons tests show a significant interaction between bin and genotype (F_{9,36}=2.412, P=0.0294). For all ages, N=3/genotype. Results from post hoc and t-tests indicated on graphs. Error bars represent mean+/−SEM. *P<0.05, **P<0.01, ***P<0.001, ****P<0.0001.

FIG. 11: Dyrk1a cHets exhibit decreased complexity of layer II/III neurons. (A) Sholl analysis of neuronal arborization on individual layer II/III pyramidal neurons shows that cHets exhibit significantly decreased neuronal arborization (independent sample t-test of area under the curve generated for each genotype: t₄=7.234, P=0.0019). N=10-15 neurons/mouse, 3 mice/genotype across 2 sections. (B) cHet layer II/III pyramidal neurons contain less complex basal branches, measured by counting the endpoints on the reconstructed neurons (Apical: t₄=3.378, P=0.027848, Basal: t₄=4.066, P=0.015269). (C) Adult cHets putative pyramidal neurons exhibit decreased cell soma size in layer II/III pyramidal neurons in the mPFC (t₄=9.835, P=0.0006). (D) cHet layer II/III pyramidal neurons exhibit more circular somas measured by the circularity plugin in ImageJ (t₄=5.282, P=0.0062). N=3/genotype. Results analyzed by independent sample t-tests unless otherwise specified. Results from post hoc and t-tests indicated on graphs. Error bars represent mean+/−SEM. *P<0.05, **P<0.01, ***P<0.001, ****P<0.0001.

FIG. 12: Proposed pathway. A schema of a proposed signaling mechanism by which mTOR and TrkB signaling are regulated in Dyrk1a mutants. Decreased Dyrk1a dosage causes increased inhibition of GSK3(3, which alone is sufficient to cause decreased proliferation and growth, but additionally causes decreased phosphorylation of its target MAPT. MAPT is known to be phosphorylated by Dyrk1a at 5202 and 5404 (14); however, the phosphopeptide identified by the proteomic experiment is 5704. MAPT is a critical regulator of microtubule dynamics and is a negative regulator of Pten. Decreased activation of MAPT would, in turn, cause increased activation of Pten and thus decreased mTOR activity, causing microcephaly and the observed decreased neuronal growth. Cdk1 targets MAPT (15) and TSC2 (16) (a negative regulator of mTOR). The inhibitory phosphorylation of Cdk1 (Y15) is increased in Dyrk1a cKOs, which would theoretically lead to decreased phosphorylation of MAPT and decreased inhibition of TSC2, resulting in decreased mTOR signaling. This was validated using western blot, as p-CDK1 (Y15) was found to be increased relative to total CDK1. Dyrk1a is known to phosphorylate Tau (14) and this phosphorylation site is decreased in the phospho-proteome. MAPT is a negative regulator of Pten (17) and thus is an interesting link between Dyrk1a and Pten/mTOR signaling.

FIG. 13: Activation of the mTOR pathway is decreased in the cortex of conditional Dyrk1a mutants. (A) P0 cKO cortices exhibit decreased activation of mTOR signaling molecules. Western blot on cortical lysate normalized to β-Actin from P0 pups analyzed by one-way ANOVA with Tukey's post hoc multiple comparisons tests (p-mTOR: F_{2,10}=12.35, P=0.002; p-S6K: F_{2,10}=6.5, P=0.0155; p-S6: F_{2,10}=15.82, P=0.0008). (B) P7 cHets exhibit decreased activation of S6K in the cortex (t₅=3.76018, P=0.0132) and a trend towards decreased activation of S6 (t₅=2.38094, P=0.063) and ERK1 (t₇=2.21842, P=0.062). Analyzed by independent sample t-tests. (C) Adult cHets do not exhibit decreased activation of mTOR relative to a housekeeping gene in the cortex. Analyzed by independent sample t-tests. Quantified using relative abundance in ImageJ. For all ages, N=3-5/genotype. Results from t-tests indicated on graphs. Error bars represent mean+/−SEM. *P<0.05, **P<0.01, ***P<0.001, ****P<0.0001.

FIG. 14: Deletion of Dyrk1a in layer V neurons is sufficient to drive a reduction in mTOR activation. (A) Breeding scheme used to generate layer V knockouts of Dyrk1a. (B) Images of the mouse cerebral cortex showing the Rbp4-cKO cortex (right) lacks enriched p-S6 staining (green) in layer V neurons (magenta). (C) Breeding scheme used to generate cortical mTOR knockouts. (D) Images of coronal brain section at birth from control (top) and mTOR cKO (bottom) showing that mTOR cKO exhibits a thin/absent corpus callosum, thin cerebral cortex, and abnormally shaped ventricles. (E) Representative image of Nissl-stained somas in P0 control and mTOR cKO cortices. (F) Quantification of soma size in all layers of the P0 cortex shows mTOR cKOs exhibit decreased soma size throughout S1 (II-IV: t₄=7.296, P=0.001876; V: t₄=12.57, P=0.000231; VI: t₄=9.835, P=0.000599). Analyzed by multiple t-tests. N=3/genotype. Results from t-tests indicated on graphs. Error bars represent mean+/−SEM. *P<0.05, **P<0.01, ***P<0.001, ****P<0.0001.

FIG. 15: Pharmacological treatment with (1-3)IGF-1 rescues decreased arborization of cHet neurons in vitro. (A) Representative neurons at 40× magnification on DIV15 treated with vehicle or (1-3)IGF-1. (B) Sholl analysis of neuronal arborization on individual neurons. Two-way ANOVA with Tukey's post hoc multiple comparisons tests on the area under the curve from the Sholl profile indicates a significant interaction between genotype and drug treatment (F_{1,8}=5.457, P=0.0477), showing that cHet pyramidal neurons exhibit significantly decreased neuronal arborization. N=30-35 neurons from 3 animals/genotype/treatment group. Results from post hoc tests indicated on graphs. Error bars represent mean+/−SEM. *P<0.05, **P<0.01, ***P<0.001, ****P<0.0001.

FIG. 16: Cellular events underlying altered postnatal brain growth trajectory in cHets. At birth, decreased cortical mass in cHets is driven primarily by decreased neuronal size. Increased apoptosis between P0 and P7 causes a decrease in the cHet cortical cell number, which is driven by a decrease in the total neuronal population. Between P7 and adult, we hypothesize that non-neuronal cells are lost due to apoptosis, causing a more severe decrease in cell number driven by a reduction in both putative neuronal and glial populations. Note that this trajectory applies to Dyrk1a cHets, and the trajectory for other models, such as germline Dyrk1a mutants, may be somewhat different due to factors such as the timing of Cre-mediated recombination or the perdurance of Dyrk1a protein.

FIGS. 17A-171 show that Chronic (1-3)IGF-1 treatment rescues cortical undergrowth in Dyrk1a cHets.

FIGS. 18A-181 show that chronic (1-3)IGF-1 treatment rescues sociability deficits in Dyrk1a cHets.

FIGS. 19A-19E show that locomotor, anxiety-like, and sensorimotor gating behaviors are unaffected by Dyrk1a mutation or (1-3)IGF-1 treatment.

FIGS. 20A-20G show that (1-3)IGF-1 treatment increases reduced protein synthesis in the mPFC of cHets.

FIGS. 21A-21F show that GSK3β is a hyperactive kinase in cHets.

FIGS. 22A-22J show that genetic suppression of GSK3β rescues microcephaly and ASD-relevant behaviors.

FIGS. 23A-23H show that genetic suppression of GSK3β rescues cell number of both neuronal and non-neuronal populations.

FIGS. 24A-24B show that anxiety-like behavior and locomotion are unaltered in cHets and dHets.

FIGS. 25A-25D show that pharmacological inhibition of GSK3β rescues microcephaly in neonatal cHets

FIGS. 26A-26D show that genetic suppression of Gsk3b rescues decreased protein synthesis in neonatal and adult cHets.

FIGS. 27A-27G is a table showing significantly altered proteins in the cKO cortex at birth.

FIGS. 28A-28P is a table showing significantly altered phosphopeptides in the cKO cortex at birth.

### DEFINITIONS

An “agonist” is a compound that activates a protein, increases activity of a protein, or increase stability of a protein compared to the activation, activity, or stability in the absence of the agonist. An agonist can be a reversible agonist or an irreversible agonist. An irreversible agonist may bind permanently to a target protein, such as through formation of a covalent bond, or chemical alteration of the target protein. The protein can be, but is not limited to, a receptor or an enzyme.

An “antagonist” or “inhibitor” is a compound that deactivates a protein, decreases (inhibits) activity of a protein, or decrease stability of a protein compared to the activation, activity, or stability in the absence of the antagonist. An antagonist can be a reversible antagonist or an irreversible antagonist. An irreversible antagonist may bind permanently to a target protein, such as through formation of a covalent bond, or chemical alteration of the target protein. The protein can be, but is not limited to, a receptor or an enzyme. An antagonist can be a competitive antagonist or a non-competitive antagonist. A competitive antagonist competes with an agonist for binding to the target protein. A non-competitive antagonist does not compete with an agonist for binding to a target protein.

A “positive effector” is a compound that activates a protein or gene, increases activity of a protein, increases expression of a gene, or increases stability of a protein or mRNA compared to the activation, activity, expression, or stability in the absence of the agonist. A positive effector can be, but is not limited to, an agonist ligand, an agonist antibody, or a transcription factor.

A “negative effector” is a compound that deactivates a protein or gene, decreases (inhibits) activity of a protein, decreases (inhibits) expression of a gene, or decrease stability of a protein or mRNA compared to the activation, activity, expression, or stability in the absence of the antagonist. A negative effector can be, but is not limited to, an antagonist ligand, an antagonist antibody, a transcription inhibitor, or a RNA function inhibitor.

A functional RNA comprises any RNA that is not translated into protein but whose presence in the cell alters the endogenous properties of the cell.

A RNA function inhibitor comprises any polynucleotide or nucleic acid analog containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. RNA function inhibitors are selected from the group comprising: siRNA, microRNA, interfering RNA or RNAi, dsRNA, RNA Polymerase II transcribed DNAs encoding siRNA or antisense oligonucleotides, RNA Polymerase III transcribed DNAs encoding siRNA or antisense oligonucleotides, ribozymes, antisense oligonucleotides, and antisense nucleic acid, which may be RNA, DNA, or artificial nucleic acid. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding RNA gene products about 22 nt long that direct destruction or translational repression of their mRNA targets. Antisense oligonucleotides comprise sequence that is complimentary to an mRNA. Antisense oligonucleotides include, but are not limited to: morpholinos, 2′-O-methyl polynucleotides, DNA, RNA and the like. RNA polymerase III transcribed DNAs contain promoters selected from the list comprising: U6 promoters, H1 promoters, and tRNA promoters. RNA polymerase II promoters include U1, U2, U4, and U5 promoters, snRNA promoters, microRNA promoters, and mRNA promoters. These DNAs can be delivered to a cell wherein the DNA is transcribed to produce small hairpin siRNAs, separate sense and anti-sense strand linear siRNAs, or RNAs that can function as antisense RNA or ribozymes.

An RNA function inhibitor may be polymerized in vitro, recombinant RNA, contain chimeric sequences, or derivatives of these groups. The RNA function inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

An RNA interference (RNAi) polynucleotide is a molecule capable of inducing RNA interference through interaction with the RNA interference pathway machinery of mammalian cells to degrade or inhibit translation of messenger RNA (mRNA) transcripts of a transgene a sequence specific manner. RNAi polynucleotides may be selected from the group comprising: siRNA, microRNA, double-strand RNA (dsRNA), short hairpin RNA (shRNA), and expression cassettes encoding RNA capable of inducing RNA interference. siRNA comprises a double stranded structure typically containing 15-50 base pairs and preferably 21-25 base pairs and having a nucleotide sequence identical (perfectly complementary) or nearly identical (partially complementary) to a coding sequence in an expressed target gene or RNA within the cell. An siRNA may have overhangs, such as dinucleotide 3′ overhangs.

An siRNA may be composed of two annealed polynucleotides or a single polynucleotide that forms a hairpin structure. An siRNA molecule of the invention comprises a sense region and an antisense region. In one embodiment, the siRNA of the conjugate is assembled from two oligonucleotide fragments wherein one fragment comprises the nucleotide sequence of the antisense strand of the siRNA molecule and a second fragment comprises nucleotide sequence of the sense region of the siRNA molecule. In another embodiment, the sense strand is connected to the antisense strand via a linker molecule, such as a polynucleotide linker or a non-nucleotide linker.

MicroRNAs (miRNAs) are small noncoding RNA gene products about 22 nucleotides long that direct destruction or translational repression of their mRNA targets. For miRNAs, the complex binds to target sites usually located in the 3′ UTR of mRNAs that typically share only partial homology with the miRNA. A “seed region”—a stretch of about seven (7) consecutive nucleotides on the 5′ end of the miRNA that forms perfect base pairing with its target—plays a key role in miRNA specificity. Binding of the RISC/miRNA complex to the mRNA can lead to either the repression of protein translation or cleavage and degradation of the mRNA. Recent data indicate that mRNA cleavage happens preferentially if there is perfect homology along the whole length of the miRNA and its target instead of showing perfect base-pairing only in the seed region (Pillai et al, 2007).

An antisense oligonucleotide (ASOs) comprises an oligonucleotide having a nucleobase sequence that is complementary to a target nucleic acid or region or segment thereof. In certain embodiments, an antisense oligonucleotide is specifically hybridizable to a target nucleic acid or region or segment thereof. ASOs can comprise nucleobase sequence and optionally one or more additional features, such as a conjugate group or terminal group. ASOs may be single-stranded and double-stranded compounds. ASOs include, but are not limited to, oligonucleotides, ribozymes, and morpholinos (peptide-conjugated phosphorodiamidate oligonucleotides (PPMOs) or simply phosphorodiamidate oligonucleotides (PMOs)). Antisense nucleic acids act by hybridization of an antisense nuclei acid to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target. Antisense nucleic acid can inhibit gene expression by reducing the levels of target RNA in a cell or by inhibiting translation, splicing, or activity of an RNA in a cell.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “complementarity” refers to the ability of a polynucleotide to form hydrogen bond(s) (hybridize) with another polynucleotide sequence by either traditional Watson-Crick or other non-traditional types. A percent complementarity indicates the percentage of bases, in a contiguous strand, in a first nucleic acid sequence which can form hydrogen bonds (e.g., Watson-Crick base pairing) with a second nucleic acid sequence (e, g., 5, 6, 7, 8, 9, 10 out of 10 being 50%, 60%, 70%, 80%, 90%, and 100% complementary). Percent complementarity is calculated in a similar manner to percent identify.

A “vector” comprises a nucleic acid sequence encoding an expression product (e.g., a peptide (i.e., polypeptide or protein such as any of the described proteins) or an RNA). A vector can be, but is not limited to, a plasmid, viral vector, a construct, or composition comprising a nucleic acid encoding the expression product. A vector may comprise one or more sequences that facilitate replication of a sequence in a cell and/or integration of a sequence into a target nucleic acid sequence. A vector may comprise one or more sequences necessary for expression of the encoded expression product in a cell. The vector may comprise one or more of: an enhancer, a promoter, intron, a terminator, and a polyA signal operably linked to the DNA coding sequence. A vector may also comprise one or more sequences that alter stability of a messenger RNA (mRNA), RNA processing, or efficiency of translation. The viral vector can be, but is not limited to, an AAV vector, an adenovirus, a retrovirus, a lentivirus, a vaccinia virus, an alphavirus, or a herpesvirus. An adeno-associated virus can be, but is not limited to, AAV1, AAV2, AAV2/1, AAV2/2, AAV2/4, AAV2/5, AAV2/6, AAV2/7, AAV2/8, AAV2/9, AAV4, AAV5, AAV6, AAV7, AAV7m8, AAV8, AAV9, and AAV44. Nucleic acid encoding the desired protein can be packaged into the viral vectors using methods and constructs known in the art. Vectors can be manufactured in large scale quantities and/or in high yield. Vectors can be manufactured using GMP manufacturing. Vectors are can be formulated to be safe and effective for injection into a mammalian subject. Vectors can be delivery to a subject, to an organ or tissue in the subject or to cells in a subject using method known in the art for gene therapy.

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339: 823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.
“ERK1/2” is ERK1 and/or ERK2.

Compositions or methods “comprising” or “including” one or more recited elements may include other elements not specifically recited.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value. Unless otherwise apparent from the context, the term “about” encompasses values within ±5% or ±10% of a stated value.

The singular forms of the articles “a,” “an,” and “the” include plural references unless the context clearly dictates otherwise.

### DETAILED DESCRIPTION

The present invention is predicated in part on the discovery by the inventors of a previously unknown mechanism through which Dyrk1a mutations that disrupt growth factor signaling in the developing brain, thus influencing neuronal growth and connectivity. As detailed below, the inventors found that cortical deletion of Dyrk1a in mice causes decreased brain mass and neuronal size, structural hypoconnectivity, and autism-relevant behaviors. Using phospho-proteomic screening, the inventors identified growth-associated signaling cascades dysregulated upon Dyrk1a deletion, including the TrkB/BDNF (brain-derived neurotrophic factor) and the mTOR signaling pathways. It was further observed by the inventors that genetic suppression of Pten or pharmacological treatment with IGF-1, both of which impinge on these signaling cascades, rescued microcephaly and neuronal undergrowth in neonatal mutants.

The invention accordingly provides novel methods for treating or ameliorating symptoms of DYRK1A associated disorders. DYRK1A associated disorders refer to any disorders or medical conditions that are associated with or mediated by dysregulation or dysfunction of DYRK1A (e.g., due to mutations in the Dyrk1a gene). Some DYRK1A associated disorders refer to conditions that are caused by loss of function mutations in the Dyrk1a gene. Notable examples of DYRK1A associated disorders include a DYRK1A syndrome, microcephaly, sociability deficits, autism spectrum disorder (ASD) and intellectual disability (ID). Various other DYRK1A associated medical conditions are also well known in the art, e.g., developmental delay, anxiety, seizures, speech and motor difficulties, and vision abnormalities (1-6). As detailed herein, the methods of the invention involve stimulating or up-regulating one or more of the signaling cascades identified herein that are suppressed or down-regulated by dysfunctional DYRK1A, e.g., mTOR and BDNF-TrkB signaling pathways. In some preferred embodiments, the subject to be treated with methods of the invention is one who has dysfunctional DYRK1A or loss of function mutation(s) in the Dyrk1a gene. In some methods, treating a DYRK1A-related disorder comprises improving sociability, decreasing microcephaly, increasing spine density, and/or improving synaptic function. Methods of the invention are useful in increasing cortical mass, cortical cell growth [cell size and/or number], neuronal cell growth [cell size and/or number], protein synthesis, and/or spinal density in a subject having a loss of function DYRK1A mutation. An increase in cortical cell growth may be an increase in cell size and/or number. An increase in neuronal cell growth may be an increase in cell size and/or number. To stimulate the signaling pathway that is disrupted or suppressed by DYRK1A dysfunction, either a positive effector or a negative effector molecule of the pathway can be targeted. For example, the methods can entail up-regulation of one or more positive effectors in these pathways. These include, e.g., BDNF, TrkB, mTOR, IGF-1, and ERK1/2.

An exemplary BDNF is characterized by an amino acid sequence of SEQ ID NO:1. An exemplary TrkB is characterized by an amino acid sequence of SEQ ID NO:3. An exemplary mTOR is characterized by an amino acid sequence of SEQ ID NO:5. An exemplary IGF-1 is characterized by an amino acid sequence of SEQ ID NO:7. An exemplary ERK1 is characterized by an amino acid sequence of SEQ ID NO:9. An exemplary ERK2 is characterized by an amino acid sequence of SEQ ID NO:11.

In some of these embodiments, up-regulation of a positive effector of these pathways can be implemented by administering the effector molecule itself or a variant or analog thereof, e.g., BDNF, TrkB, mTOR, ERK1/2, IGF-1, or the (1-3)IGF-1 tripeptide as exemplified herein. In some embodiments, the positive effector of IGF-1 comprises Trofinetide (IGF-1 synthetic peptide analog AKA NNZ-2566) or NNZ-2591.

In some embodiments BDNF protein is delivered systemically. In some of these embodiments, up-regulation of a positive effector of these pathways can be implemented by administering a nucleic acid encoding BDNF, TrkB, mTOR, IGF-1 ERK1, or ERK2. In some embodiments a nucleic acid encoding BDNF is delivered by a viral vector. In some of these embodiments, the nucleic acid encoding BDNF, TrkB, mTOR, IGF-1 ERK1, or ERK2 comprises a plasmid, a viral vector, or an mRNA. An exemplary nucleotide sequence encoding BDNF is SEQ ID NO:2. An exemplary nucleotide sequence encoding TrkB is SEQ ID NO:4. An exemplary nucleotide sequence encoding mTOR is SEQ ID NO:6. An exemplary nucleotide sequence encoding IGF-1 is SEQ ID NO:8. An exemplary nucleotide sequence encoding ERK1 is SEQ ID NO:10. An exemplary nucleotide sequence encoding ERK2 is SEQ ID NO:12.

In some embodiments, up-regulation of a positive effector of these pathways can be achieved via using a small molecule agonist, e.g., BDNF/TrkB agonist 7,8-dihydroxyflavone. In some embodiments, the positive effector of TrkB comprises deoxygedunin. In still some embodiments, up-regulation of the positive effector can be performed with an antibody agonist, e.g., any of the TrkB agonist antibodies well known in the art. See, e.g., Guo et al., Neurobiol. Dis., 132 (2019), Article 104590, for example LM22A-4, HIOC, R7, R13, AS86, Ab1A01, Ab104, Ab4B19, Ab2C03, Ab303, Ab1104, or Ab2908.

In some other methods, down-regulation of a negative effector or inhibitor of these pathways can be pursued. In some other embodiments, suppression of the negative effector can be performed with an inhibitory polynucleotide molecule, for example siRNA, microRNA, interfering RNA or RNAi, dsRNA, RNA Polymerase II transcribed DNAs encoding siRNA or antisense oligonucleotides, RNA Polymerase III transcribed DNAs encoding siRNA or antisense oligonucleotides, ribozymes, antisense oligonucleotides, and antisense nucleic acid, which may be RNA, DNA, or artificial nucleic acid. In some embodiments, RNAi, CRISPR, or an agonist antibody is used to suppress inhibitors of mTOR, S6K1, or ERK1/2 signaling. An exemplary S6K1 is characterized by an amino acid sequence of SEQ ID NO:133. An exemplary nucleotide sequence encoding S6K1 is SEQ ID NO:134.

For example, as exemplified herein, the methods can involve suppression or inhibition of Gsk3β. An exemplary Gsk3β is characterized by an amino acid sequence of SEQ ID NO:129. An exemplary nucleotide sequence encoding Gsk3β is SEQ ID NO:130. The suppression can be achieved, e.g., via genetic manipulation or chemical inhibition. A number of Gsk3β inhibitors known in the art can be readily employed in these methods. See, e.g., F Eldar-Finkelman Hagit and Martinez Ana, Front. Mol. Neurosci., 2011, Volume 4, Article 4. In some embodiments, the Gsk3β inhibitor comprises lithium, zinc, tungstate, an indirubin, 6-BIO, hymenialdisine, dibromocantharelline, a meridianin, an aminopyrimidine, CT98014, CT98023, CT99021, TWS119, an arylindolemaleimide, SB-216763, SB-41528, a thiazole, AR-A014418, AZD-1080, a paullone, kenpaullone, alsterpaullone, cazpaullone, an aloisine, a manzamine, manzamine A, a furanosesquiterpene, palinurine, tricantine, a thiadiazolidindione, TDZD-8, NP00111, NP031115, NP031112 (tideglusib), a halomethylketone, HMK-32, or L803-mts. In some embodiments, the Gsk3β inhibitor comprises SB216763. In other embodiments, the Gsk3β inhibitor comprises SAR502250, indirubin-3-oxime, alsterpaullone. CHIR-99021, AT7519, TWS119, indirubin, SB415286, CHIR-98014, or Tideglusib. In some embodiments, the Gsk3β inhibitor comprises an antisense oligonucleotide or an siRNA wherein the antisense oligonucleotide or the siRNA targets the Gsk3β gene and causes a decrease in expression of the Gsk3/3 gene.

For example, as exemplified herein, the methods can involve suppression or inhibition of Pten, a negative regulator of the mTOR pathway. An exemplary Pten is characterized by an amino acid sequence of SEQ ID NO:131. An exemplary nucleotide sequence encoding Pten is SEQ ID NO:132. The suppression can be achieved, e.g., via genetic manipulation or chemical inhibition. In some embodiments, a chemical inhibitor agent can be used to down-regulate Pten. A number of small molecule Pten inhibitors known in the art can be readily employed in these methods. See, e.g., Borges et al., Regenerative Med. 2020, Vol. 15, No. 2; and Pulido, Molecules. 2018 February; 23(2): 285. In some embodiments, the Pten inhibitor comprises bpV(phen), bpV(pic), bpV(HOpic), bpV(pis), VO-OHpic, or SF1670. In some other embodiments, suppression of the negative effector can be performed with an inhibitory polynucleotide molecule, e.g., siRNA agents targeting Pten. In some embodiments, the Pten inhibitor comprises an antisense oligonucleotide or an siRNA wherein the antisense oligonucleotide or the siRNA targets the Pten gene and causes a decrease in expression of the Pten gene.

In pharmacology, a route of administration is the path by which a drug, fluid, or other substance is brought into contact with the body. In general, methods of administering drugs and nucleic acids for treatment of a mammal are well known in the art and can be applied to administration of the described compounds and compositions. The described compounds and compositions can be administered via any suitable route in a preparation appropriately tailored to that route. In general, any suitable method recognized in the art for delivering a therapeutic drug, protein, or nucleic acid can be adapted for use with a herein described compounds and compositions. Routes of administration include, but are not limited to, parenteral, local, direct injection, intraparenchymal, intratumoral, intramuscular, implantation, topical, systemic, intravascular, intravenous, intra-arterial, intraventricular, intralymphatic, transdermal, intracutaneous, intradermal, subdermal, subcutaneous, intraperitoneal, intracranial, subdural, intrathecal, epidural, rectal, airway (aerosol), nasal, oral, buccal (mouth/cheek), and sublingual (under the tongue) administration. In some embodiments, the compositions are administered by subcutaneous or intravenous infusion or injection.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients, or auxiliaries. The formulation depends on the route of administration chosen.

A polynucleotide can be delivered by a viral vector system. A number of viral vector systems can be used including retroviral systems (see, e.g., Lawrie and Tumin, Cur. Opin. Genet. Develop. 3, 102-109 (1993)) including retrovirus derived vectors such MMLV, HIV-1, and ALV; adenoviral vectors {see, e.g., Bett et al, J. Virol. 67, 591 1 (1993)); adeno-associated virus vectors {see, e.g., Zhou et al., J. Exp. Med. 179, 1867 (1994)), lentiviral vectors such as those based on HIV or FIV gag sequences, viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see U.S. Pat. No. 5,643,576) and rhabdoviruses, such as vesicular stomatitis virus (see WO 96/34625) and papillomaviruses (Ohe et al., Human Gene Therapy 6, 325-333 (1995); Woo et al, WO 94/12629 and Xiao & Brandsma, Nucleic Acids. Res. 24, 2630-2622 (1996)).

CRISPR systems can be used to modify or mutate of one or more target loci (genes) in a cell. A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the cell in the form of a protein or a nucleic acid (DNA or RNA) encoding the Cas protein (e.g., operably linked to a promoter expressible in the cell). The guide RNA can be introduced into the cell in the form of RNA or a DNA encoding the guide RNA (e.g., operably linked to a promoter expressible in the cell). In some embodiments, the CRISPR system can be delivered to a cell via a viral vector.

A CRISPR system is designed to target one or more of the described target genes. The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

Suitable guide sequences include 17-20 nucleotide sequences complementary to a target sequence that are unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ in a target gene or a complement thereof can be used in forming a gRNA. In some embodiments, the guide sequence is 100% complementary to the target sequence. In some embodiments, the guide sequence is at least 90% or at least 95% complementary to the target sequence. In some embodiments, the guide sequence contains 1 or 2 mismatches when hybridized to the target sequence. In some embodiments, a mismatch, if present, is located distal to the PAM, in the 5′ end of the guide sequence.

Unless otherwise specified herein, the materials and reagents required for practicing the invention, e.g., the encoding polynucleotides, expression vectors and host cells, as well as the related therapeutic methods, can all be generated or performed in accordance with the procedures exemplified herein or routinely practiced protocols well known in the art. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Sambrook et al., Molecular Cloning: A Laboratory Manual, Cold Spring Harbor Press, N.Y., (3^{rd} ed., 2000); Brent et al., Current Protocols in Molecular Biology, John Wiley & Sons, Inc. (ringbou ed., 2003); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the compositions and methods of the present invention.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000).

The invention is exemplified in and fully supported by the specific studies set forth in the Examples below. Specifically, utilizing both heterozygous and homozygous conditional mutants has enabled the inventors to study a clinically relevant model with similar construct validity to patients and a loss-of-function model to investigate the role of Dyrk1a, respectively. The investigation of cKOs is particularly interesting because germline Dyrk1a knockouts (Dyrk1a^{-/-}) die by embryonic day 13.5 (13). As described herein, we examined the altered trajectory of postnatal brain growth and found that cortical Dyrk1a mutations cause decreased brain and cortex mass at birth, during development, and in adulthood. cHets also exhibit cortical thinning and enlarged ventricles, which may be explained by the excessive cell death observed. Interestingly, MRI reveals patients with DYRK1A mutations also display enlarged ventricles and a hypoplastic corpus callosum (4). In addition to morphological aberrations, mouse models of Dyrk1a mutations exhibit behavioral abnormalities, including altered social behavior (14, 15, 46, 47), and our finding of altered social interest in cHets illustrates the necessity of Dyrk1a in the cortex for encoding this behavior. Thus, we have shown that the conditional Dyrk1a mutant accurately models phenotypes observed in humans with DYRK1A mutations in both brain size/morphology and ASD-relevant behavioral deficits.

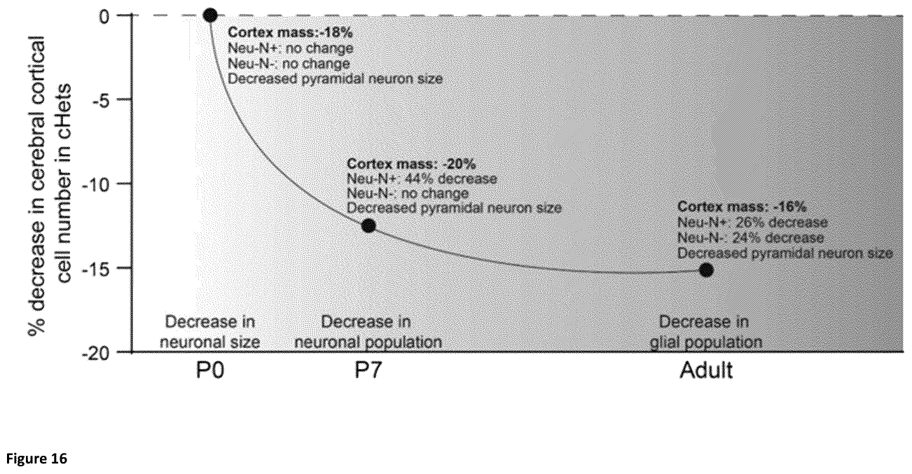

Dyrk1a plays an evolutionarily conserved role in regulating proliferation in neural progenitor cells in both vertebrates and invertebrates (48-50). Thus, we were surprised that cortical cell number was not changed in cHets or cKOs as compared to controls at birth. This may differ from findings in germline Dyrk1a^{+/-} mutants due to the timing of Dyrk1a deletion using Emx1-Cre. However, germline Dyrk1a^{+/-} mutants exhibit increased density in the cortex, which suggests the presence of smaller cells (13). This supports our finding of decreased cell size at all ages in the cHet and cKO cortex. Layer V pyramidal neurons are one of the largest neuronal types in the brain (51), send subcortical projections that are critical for social behavior and other ASD-relevant behaviors (52, 53), and their size is correlated with the amount of mTOR activity in the neuron (20). The decreased complexity of layer V neurons in the mPFC together with the decrease in neuronal number and layer V (Ctip2+) neuronal density suggest potential cellular mechanisms responsible for altered connectivity and behavior in Dyrk1a mutants. A proposed cellular mechanism driving the altered postnatal growth trajectory in cHets is summarized in FIG. 16.

Using high-throughput proteomics and phospho-proteomics, we identified signatures of altered growth, development, and microtubule dynamics in cKOs at birth. The role of Dyrk1a in regulating microtubule dynamics has been described (54), but implication of both BDNF-TrkB signaling and mTOR signaling is novel. The nature of the interaction between Dyrk1a and BDNF remains unclear; however, our findings provide a mechanism by which Dyrk1a regulates survival and growth through BDNF-TrkB signaling. It is important to note that mutations in TrkB cause decreased cellular size and neuronal complexity as well as a compressed cortex (55). TrkB is a critical receptor that activates multiple growth signaling cascades, suggesting changes in activation of TrkB by Dyrk1a will result in alterations in plasticity through CAMK2 and PKC, transcription and translation through AKT/mTOR, and growth and differentiation through ERK (56). Additionally, Xu et al. noted “rounder” cell somas in cortical pyramidal neurons of TrkB mutants, supporting our finding that Golgi-stained pyramidal neurons in cHets exhibit more circular cell somas (55).

Concordant with the predicted involvement of mTOR signaling in Dyrk1a mutants by IPA, p-S6 levels are decreased in layer V neurons at all ages in the conditional Dyrk1a mutant, and mTOR cKO phenocopies the pyramidal neuronal undergrowth observed in Dyrk1a cKOs. While these data underscore a novel role of mTOR dysregulation in Dyrk1a mutants, our findings cannot rule out other contributing mechanisms. Signaling through the mTOR pathway is critical for regulating neuronal growth via protein synthesis and controlling proliferation, differentiation, migration, and autophagy in diverse neuronal cell types (28, 57). Deviations from optimal levels of protein synthesis—either increased or decreased—are detrimental for synaptic connectivity and cognitive function (58). Genetic suppression of Pten in cHets not only rescued the observed microcephaly and decreased neuronal growth but also provided evidence of a novel genetic interaction between two highly penetrant ASD/ID risk genes that impact brain growth. We also utilized (1-3)IGF-1 treatment, which has been used in Shank3 and Mecp2 mutant mouse models of ASD to rescue growth and behavioral deficits (45, 59, 60). To our knowledge, this is the first investigation of the efficacy of an FDA-approved drug (Increlex, FDA reference ID 3517143) to rescue neurodevelopmental deficits caused by Dyrk1a haploinsufficiency. Neonatal treatment with IGF-1 rescued brain and neuronal undergrowth in neonatal cHets, corresponding with an increase in p-S6 levels. Additionally, (1-3)IGF-1 treatment rescued neuronal complexity in primary neurons in vitro, suggesting the potential to rescue altered connectivity and behavior in vivo.

In summary, the exemplified studies identified Dyrk1a as a gene that impinges on growth-associated signaling cascades, including BDNF-TrkB and mTOR. These studies revealed a novel molecular mechanism through which mutations in Dyrk1a cause microcephaly and decreased neuronal size via decreased growth factor signaling and tested a therapeutically relevant avenue to rescue the observed deficits.

Loss-of-function mutations in DYRK1A, a gene in the down syndrome critical region of chromosome 21, cause autism spectrum disorder (ASD) and intellectual disability (ID) in addition to macroscale and microscale alterations in the morphology of the brain. As many as 90% of patients with DYRK1A mutations exhibit primary microcephaly and this often coincides with morphological characteristics including hypomyelination of the corpus callosum and enlarged ventricles and behavioral characteristics including anxiety and severe speech delay (IA-6A). Previous work showed that Dyrk1a mutations cause cortical undergrowth via decreased cellular size at birth and a postnatal decrease in cellular number in mice (7A). This, combined with decreased complexity of subcortical projection neurons and decreased cortico-subcortical connectivity results in ASD-relevant behaviors in these conditional mutant mice including decreased sociability.

Dyrk1a kinase activity impacts several targets including Forkhead Box 01 (FOXO1), P53, Cyclin Dependent Kinase Inhibitor 1B (CDKN1B/P27Kip1), and Glycogen Synthase Kinase 3 Beta (GSK3β)(8A-12A). In addition to direct inactivation of GSK3β, Dyrk1a influences downstream signaling of GSK3β targets by acting as a priming kinase(13A-16A). Through these targets, Dyrk1a regulates proliferation, differentiation, and cell cycle exit in the developing mammalian brain. It was previously unknown whether Dyrk1a directly targets effectors within mTOR/growth signaling cascades and how this impacts overall brain growth and connectivity. Mutations in Dyrk1a result in altered activation of ERK/Akt and mTOR signaling has been shown to be dysregulated in postmortem brain tissue of patients with DYRK1A mutations, however no causative link between Dyrk1a mutations and altered growth factor signaling had been identified(17A-19A). Using mass spectrometry proteomic analysis of the Dyrk1a mutant cortex, we identified a signature of altered growth signaling and validated our findings at both the cortical and neuronal level through alteration of mTOR signaling cascade components. Signaling downstream of receptor tyrosine kinases (RTKs) Insulin-like Growth Factor 1 (IGF-1) and Tropomyosin Receptor Kinase B (TrkB) regulates critical cellular processes including growth and protein synthesis (20A-23A). Moreover, dysregulated protein synthesis occurs in a number of autism models and has been proposed to be a convergent pathogenic mechanism for a number of ASD etiologies (24A-28A). Thus, we are testing the hypothesis that Dyrk1a mutations cause altered protein synthesis as a pathogenic mechanism of undergrowth downstream of mTOR signaling.

Currently, there are no therapeutic strategies being tested for patients with DYRK1A mutations and identification of potential translational strategies is difficult due to the narrow critical period that occurs early in development and has been suggested to have exacerbated restriction in children with ASD (29). IGF-1 is an FDA-approved drug (Increlex) which has been shown to safely and effectively treat short stature in children (30A). IGF-1 and its active peptides ameliorate brain undergrowth and other deleterious phenotypes caused by Mecp2 mutations in mice (31A, 32A). Furthermore, we have shown that neonatal treatment of Dyrk1a conditional mutant mice with the active peptide (1-3)IGF-1 rescues microcephaly through increased neuronal size and mTOR activity and (1-3)IGF-1 treatment normalized reduced neuronal complexity caused by Dyrk1a mutations in vitro. Taken together, there is promising therapeutic potential for (1-3)IGF-1 in ameliorating undergrowth in Dyrk1a mutant mice; however, it is not yet known whether (1-3)IGF-1 treatment rescues microcephaly and ASD-relevant behavior in adult Dyrk1a mutant mice. As we are testing whether altered protein synthesis is a pathogenic mechanism caused by Dyrk1a mutations, it is also critical to determine whether this is ameliorated by treatment with (1-3)IGF-1.

Loss-of-function mutations in DYRK1A are a strong genetic cause of ASD, ID, and microcephaly in humans (1A-3A, 5A, 6A). Similarly, a number of genetic mutations that cause neurodevelopmental disorders converge on undergrowth of the head and brain including Mecp2, Slc9a6, Ube3a, and Ranbpl, and studies of these genetic determinants inform multiple points of convergence in the underlying pathogenic mechanisms (43A-47A). One commonly posited point of convergence amongst genetic mutations that impact brain growth is that they do so through targeting of mTOR signaling and/or protein translation (25A, 28A, 48A). Previous findings from the lab indicate that not only is mTOR/growth factor signaling decreased, but that administration of an active peptide of IGF-1 ((1-3)IGF-1) rescues cortical and neuronal undergrowth in cHets. Moreover, (1-3)IGF-1 has been shown to have beneficial effects in another mouse model of neurodevelopmental disorders and is currently FDA-approved to treat children with short stature (Increlex)(30A, 32A). Here, we find that chronic administration of (1-3)IGF-1 rescues microcephaly and sociability deficits in cHets without negatively impacting control mice. We find that the cellular correlate of normalized cortical mass is rescued neuronal complexity and spine density, both of which are decreased in cHets. This promising therapeutic effect is advantageous not only for application toward DYRK1A mutations, but potentially MECP2 mutations as well. We were surprised to find that chronic administration beginning at P14 was sufficient to rescue undergrowth and behavioral deficits, as this timepoint occurs after proliferation, neurogenesis, and synaptogenesis (49A-51A). In support of this later developmental treatment, the same administration paradigm was utilized in a mouse model of Rett syndrome and chronic administration of (1-3)IGF-1 significantly improved lifespan, motor coordination, cardiac function, microcephaly, spine density, and synaptic function (32A). Conversely, several mouse models of overgrowth or hyperconnectivity show a failure to rescue these phenotypes when treatment is begun after early development, including Pten, Tscl, Nfl, and Fmrl mutations (27A, 52A-55A). From this data, we suggest that models of undergrowth are less sensitive to critical periods than models of overgrowth. Importantly, we have identified a potential therapeutic for DYRK1A syndrome and elucidated that the rescue by (1-3)IGF-1 is dependent on mTOR and S6K1. A limitation of the current study is the reduction of body mass by co-administration of (1-3)IGF-1 and Rapamycin. This suggests that for therapeutic efficacy, S6K1 may be a more advantageous target compared to mTOR.

Through high-throughput phosphoproteomics, bioinformatic analysis, and direct probing of cortical lysate, we identified GSK30 as a hyperactive kinase responsible for altered targeting of downstream phosphopeptides in the Dyrk1a mutant cortex. Furthermore, Dyrk1a and GSK3β are highly correlated in the developing human brain transcriptome, as identified via Brainspan (Allen Institute for Brain Science). Dyrk1a directly targets and inhibits GSK3β at T356 and Dyrk1a acts as a priming kinase for GSK3β, thereby influencing signaling downstream of GSK3β kinase activity (9A, 13A, 14A, 16A). Furthermore, GSK3β activity is required for protein synthesis through phosphorylation of eIF2B, as inhibition of GSK3β results in increased protein synthesis and this is blocked by the additional silencing of p-eIF2B (56A). Lastly, mice with constitutively active GSK3β exhibit microcephaly, phenocopying the undergrowth we observe in Dyrk1a mutant mice (57A). Genetic suppression of Gsk3β rescued cortical and neuronal undergrowth as well as sociability deficits and decreased spine density in cHets. Moreover, pharmacological administration of GSK3 inhibitor SB216763 resulted in normalized cortex mass and cellular number in neonatal cHets. Future studies will evaluate whether chronic administration of SB216763 is sufficient to ameliorate microcephaly and ASD-relevant behaviors in adult cHets. Altogether, we have identified two novel therapeutic approaches toward DYRK1A syndrome.

### EXAMPLES

### Example 1. Conditional Mutations in Dyrk1a Cause Microcephaly and ASD-Relevant Behaviors

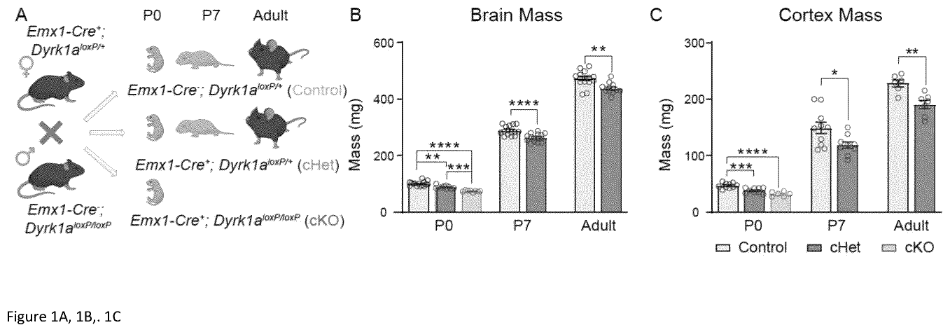

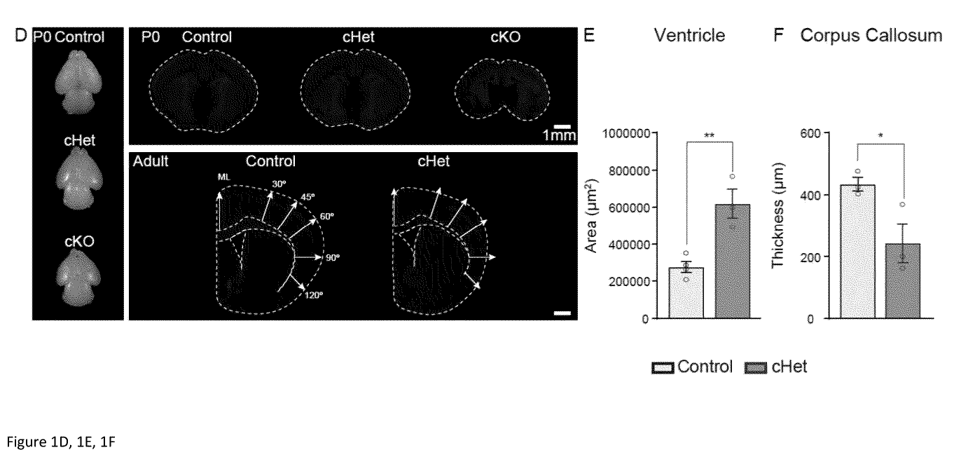

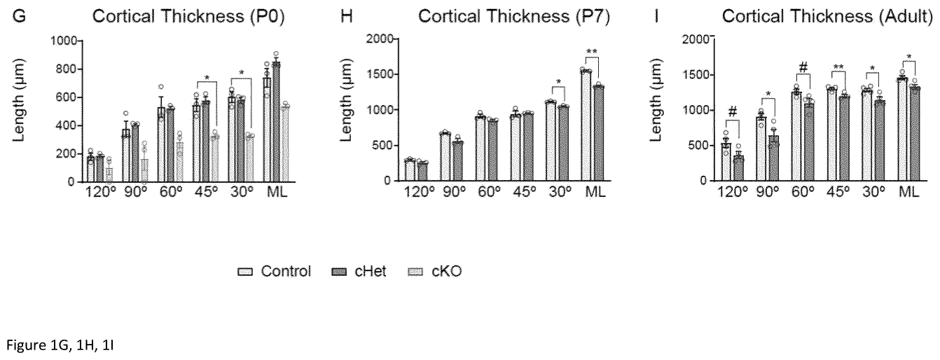

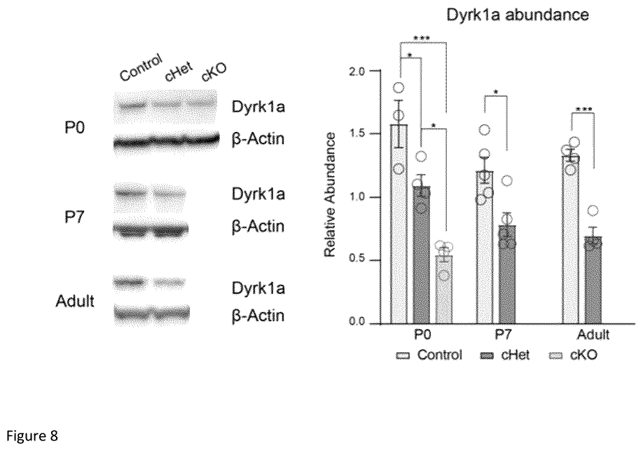

To circumvent pleiotropic effects outside the brain and isolate the effects of Dyrk1a mutation in the cerebral cortex, which is heavily impacted in microcephaly, we employed a conditional approach. Using an Emx1-cre driver, which is expressed in −88% of neurons in the neocortex and hippocampus as well as a subset of cells in the astrocyte and oligodendrocyte lineages (34), we generated heterozygous (Emx1-cre⁺; Dyrk1a^{loxP/+}, “cHet”) and homozygous (Emx1-cre⁺; Dyrk1a^{loxP/loxP}, “cKO”) mutants (FIGS. 1A, 8). cKOs survive to birth but die by the end of postnatal day 0 (P0). Thus, cKO data was only collected at P0 for all assays. We found that cHets and cKOs exhibit decreased brain and cortex mass at birth (P0), throughout development (P7), and in adulthood (>P56) (FIGS. 1B, C) indicating that cortical mutations in Dyrk1a cause microcephaly. cKOs exhibit morphological abnormalities including increased ventricle size, lack of a corpus callosum, and an absent lateral cortex (FIG. 1D, top panel). Adult cHets also exhibit significantly enlarged ventricles and a thinner corpus callosum (FIG. 1E, F). We measured cortical thickness as the distance from the ventricle to pial surface at several positions on the mediolateral axis (FIG. 1D (bottom panel)) and found that the cHet cortex is not altered at birth. However, the cKO cortex is markedly thinner (FIG. 1G). P7 and adult cHets exhibit decreased cortical thickness at multiple measurement points (FIGS. 1H, I).

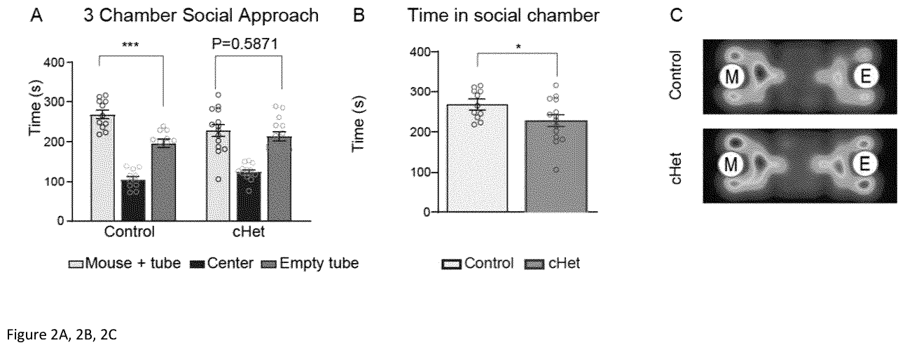

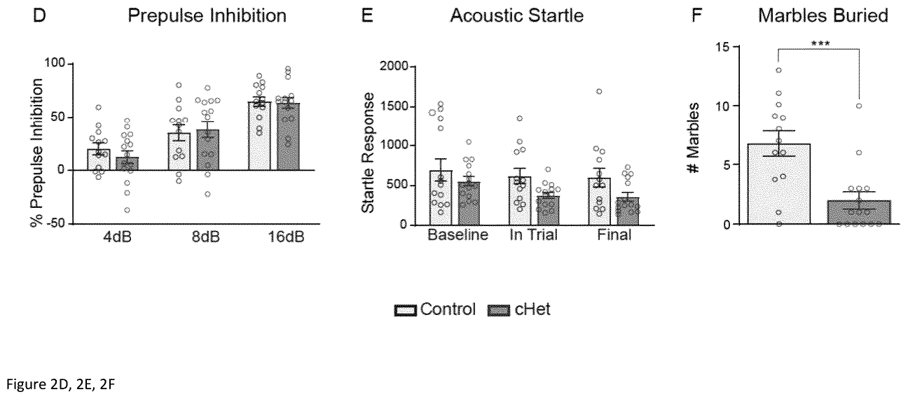

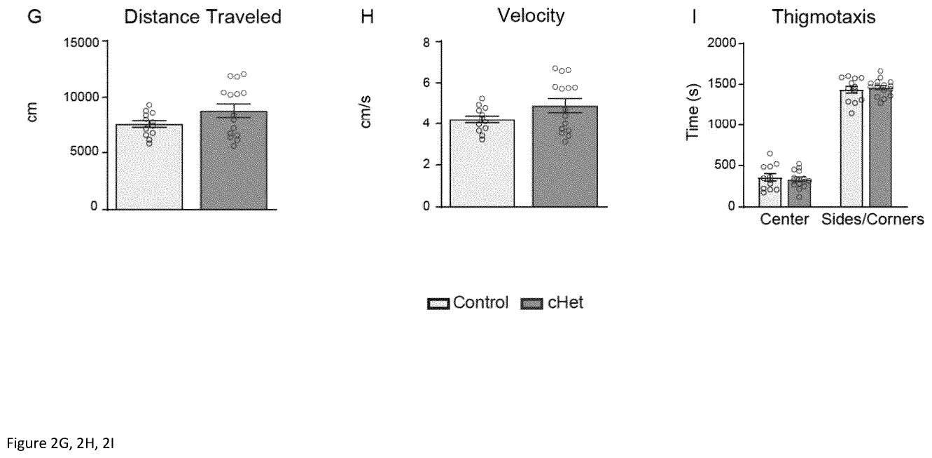

To determine whether cHets model phenotypes relevant to patients with DYRK1A mutations, we investigated ASD-relevant behaviors. In the three-chamber social approach assay, control mice exhibit a significant preference for interaction with a stimulus mouse, while cHets spend equal amounts of time in the chamber containing a stimulus mouse and the chamber with an empty tube (FIG. 2A). cHets spend significantly less time in the social chamber, suggesting a social deficit (FIGS. 2B, C). Further, cHets bury significantly fewer marbles thereby displaying an altered repetitive behavior (FIG. 2F). cHets do not exhibit alterations in sensorimotor gating (FIGS. 2D, E), locomotion (FIGS. 2G, H), anxiety-like behavior (FIGS. 2I-K), or depression-like behavior (FIG. 2L). These data show that a cortical Dyrk1a mutation is sufficient to cause microcephaly and ASD-relevant behavioral deficits.

### Example 2. Microcephaly in Dyrk1a Conditional Mutants Corresponds to Reduced Cell Size at Birth and Fewer Cells During Postnatal Development

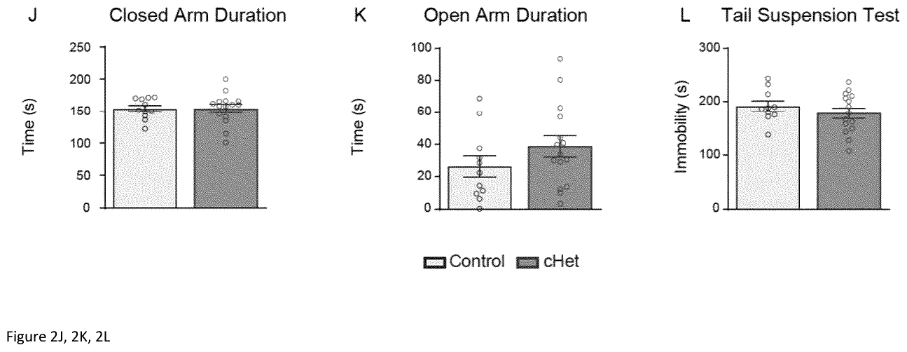

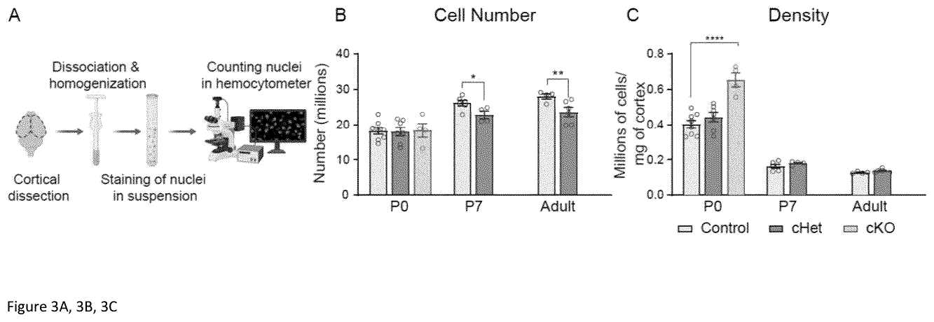

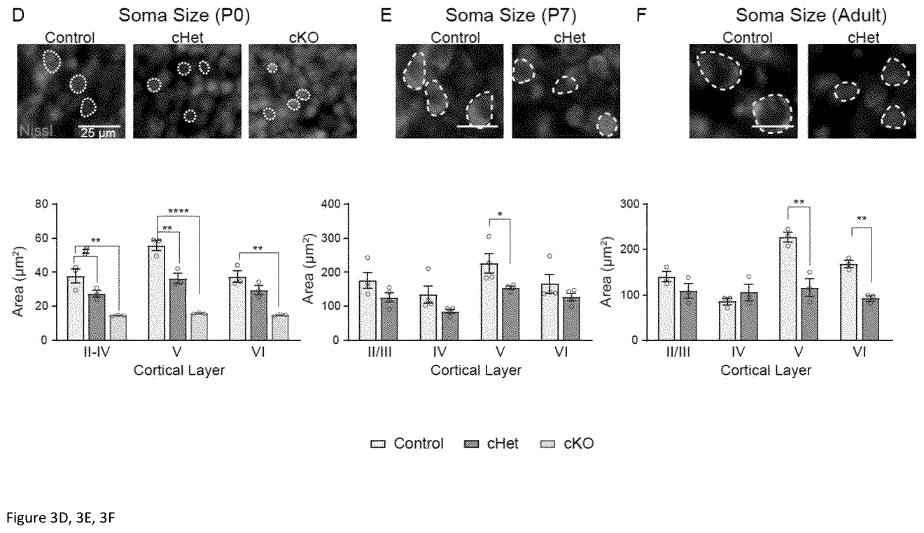

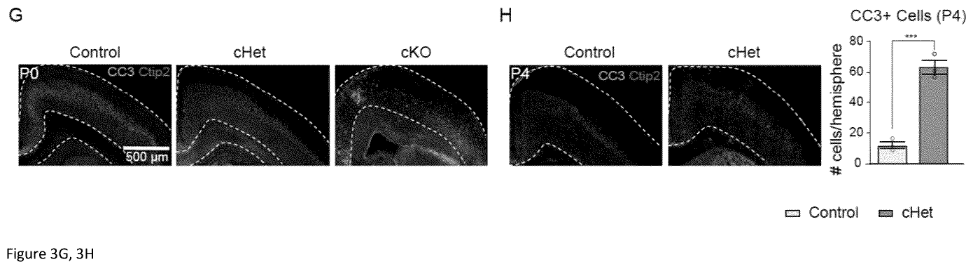

Since germline Dyrk1a^{+/-} mice exhibit altered neurogenesis and cell cycle exit (9), we predicted that decreased cell number would correspond with microcephaly at birth. We conducted isotropic fractionator to obtain the absolute number of cells (FIG. 3A) (35). Despite decreased cortical mass and thickness upon Dyrk1a deletion, cell number is unexpectedly not altered in cHets or cKOs at birth. The density of cells in the cKO cortex is significantly increased, suggesting a decrease in cell size or spacing. P7 cHets exhibit decreased cell number relative to both controls and P0 cHets, but cell density is not altered. We observed the same effect in adult cHets, with the mutant cortex containing significantly fewer cells and unchanged cellular density (FIGS. 3B, C). These findings suggest that decreased cell number is not driving microcephaly in conditional mutants at birth, but rather there is a decrease in cell number in the mutants between P0 and P7.

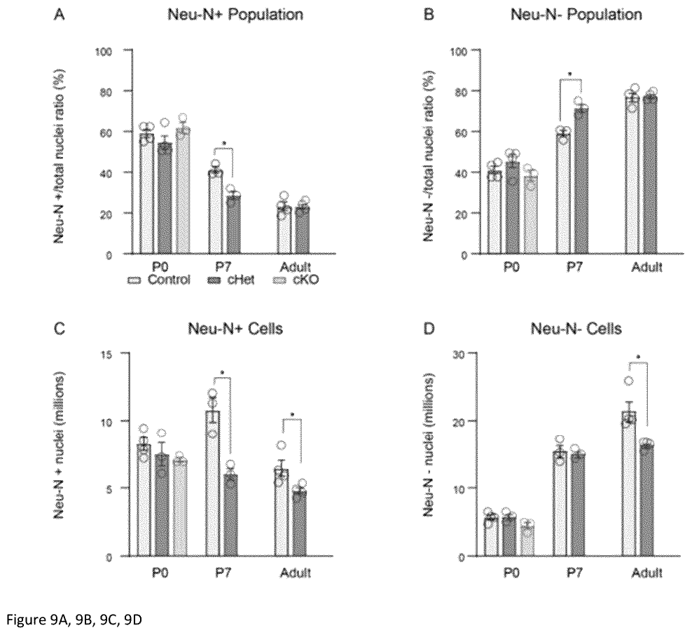

Because Emx1 is expressed in both neurons and glia, we investigated the specific cellular population driving the decreased cell number by flow cytometry of dissociated nuclei stained with neuronal marker Neu-N. At P0, in which no difference in cHet total cortical cell number is observed, the proportions (percentage of total nuclei) of Neu-N+ and Neu-N− cells are unchanged in cHets and cKOs, consistent with unchanged total numbers of both neuronal and non-neuronal populations. At P7, the decrease in cHet total cortical cell number corresponds with a significant decrease in the proportion of Neu-N+ cells and a significant increase in the proportion of Neu-N− cells, consistent with a decrease in the total number of neurons and no change in the total number of non-neuronal cells. In adults, the decrease in cHet total cortical cell number corresponds with no change in the proportion of Neu-N+ or Neu-N− cells, consistent with a decreased total number of both neuronal and non-neuronal cells. Altogether, the data show that at P0, the populations of putative neurons and putative glia are unchanged. At P7, the observed decrease in cell number may be driven by a decrease in the neuronal population, which persists into adulthood with the addition of a decrease in the putative glial population (FIG. 9).

To determine the source of increased cell density in cKOs at birth, we employed fluorescent Nissl staining to measure cell soma size in the somatosensory cortex (Si). We observed a decrease in cell soma size across all layers in cKOs at birth, aligning with our finding of increased cell density (FIG. 3D). At P0, cHets exhibit decreased cell soma size in layers II-IV and V. At P7, cell somas in layer V are significantly smaller, and this decrease persists into adulthood in addition to a significant decrease in cell soma size in layer VI (FIGS. 3E, F). This decrease of cortical cell number in cHets at P7 but not at birth prompted us to investigate cell death as a potential mechanism. We observed increased apoptosis, as measured by apoptotic marker cleaved caspase 3 (CC3), throughout the cortex at P4 (FIG. 3H). Staining of the P0 cKO cortex revealed a marked induction of apoptosis, particularly near the midline (FIG. 3G).

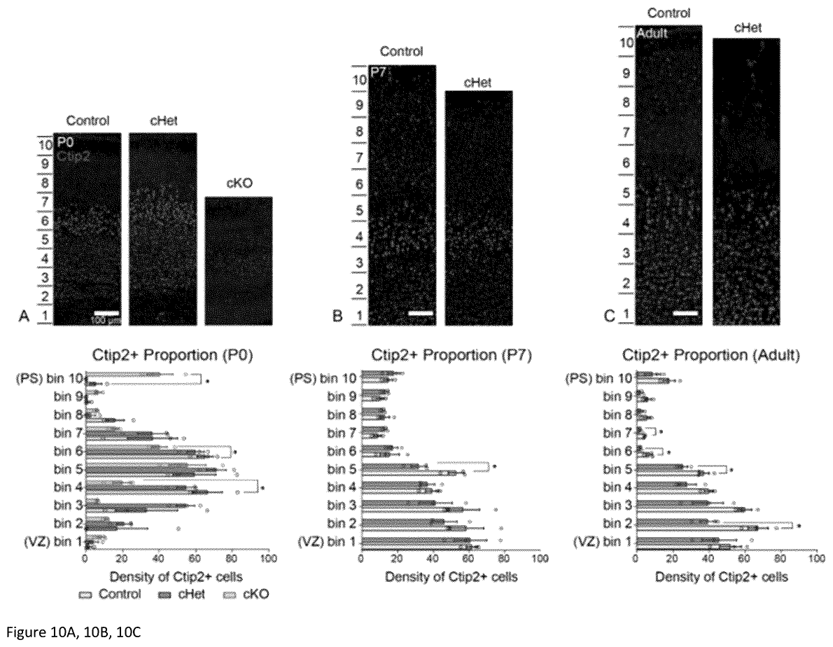

Because layer V soma size is impacted in Emx1-cre cHets and cKOs across ages, we tested whether the putative layer V (Ctip2+) population is altered in density in cHets and cKOs. We quantified the number of Ctip2+ cells normalized to DAPI+ cells using 10-bin analysis and found that at P0, the density of Ctip2+ cells is comparable in cHets but altered in cKOs (FIG. 10A). At P7 and adulthood, cHets exhibit decreased density of Ctip2+ neurons in the bins corresponding to layer V (at P7) and layers V and VI (in adults) (FIGS. 10B, C). These data indicate that conditional Dyrk1a mutations cause decreased neuronal cell number by P7 and provide layer V as a potential neuronal population driving this decrease.

Dyrk1a (RNA (36) and protein (37)) is enriched in, but is not exclusive to, layer V pyramidal neurons. This, together with decreased layer V soma size and Ctip2+ cell density at multiple timepoints in cHets led us to investigate the sufficiency of layer V-specific deletion of Dyrk1a to drive these cellular phenotypes. We generated conditional knockouts using Rbp4-Cre, which depletes Dyrk1a in layer V from development (Rbp4-cre⁺; Dyrk1a^{loxP/loxP}) (FIG. 3I). At P0, Rbp4-cKOs exhibit decreased soma size in layer V and a subtle yet significant decrease in layer VI soma size (FIGS. 3J, K). We also observed a robust induction of apoptosis within layer V of Rbp4-cKOs (FIG. 3L). Altogether, microcephaly in Dyrk1a mutants corresponds to smaller cells at birth and fewer cells after birth, which may be driven by induction of apoptosis. Additionally, deletion of Dyrk1a in layer V neurons is sufficient to drive both the reduction in cell soma size and increased apoptosis.

### Example 3. Cortical Pyramidal Neurons Display Altered Morphology and Axonal Projections Upon Dyrk1a Mutation

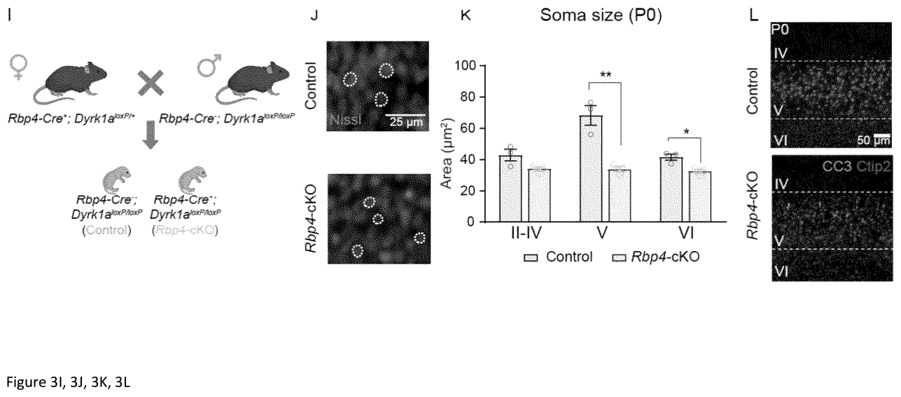

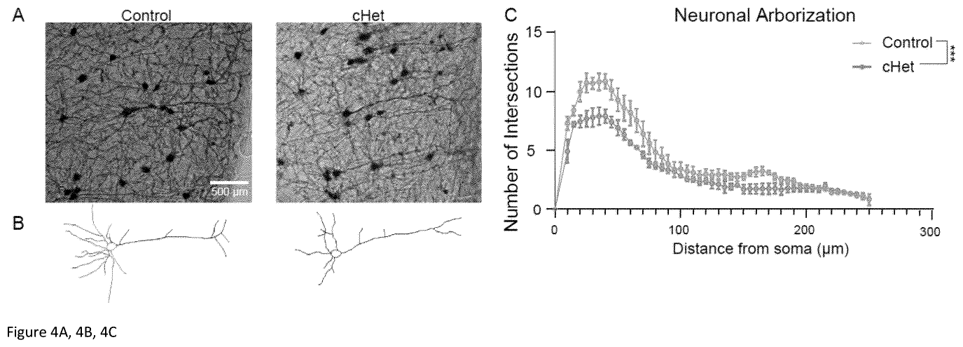

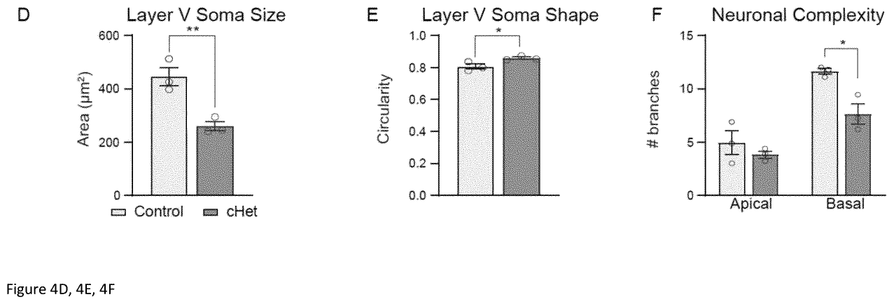

To investigate a potential cellular mechanism driving altered social behavior, we investigated whether cHets display altered neuronal complexity in vivo by measuring Golgi-stained layer V neurons in the mPFC. These neurons send projections to subcortical regions (i.e., BLA, VTA) and are responsible for encoding social behavior and other ASD-relevant behaviors (38). We found that cHets exhibit significantly less complex layer V neurons, particularly close to the soma (FIGS. 4A-C). This significant decrease in soma size of cHet neurons (FIG. 4D) validates our findings from fluorescent Nissl staining (FIG. 3F). Additionally, basal dendritic branching is significantly decreased, while apical dendrites are unaffected (FIG. 4F). After observing that somas of layer V neurons in mutant brains were more circular than controls, we quantified circularity [4π (area/perimeter²)] where 1.0 indicates a perfect circle. We found that cHet somas are significantly more circular than controls, exhibiting a mean value closer to 1.0 (FIG. 4E). A similar decrease in complexity and soma size as well as an increase in circularity of layer II/III pyramidal neurons suggests that cortical mutations in Dyrk1a have a widespread impact on neuronal morphology and axonal projections (FIG. 11).

We next investigated a circuit that encodes social behavior and is dependent on layer V neurons. Using an anterograde Cre-dependent virus (rAAV2-Ef1a-DIO-EYFP), we traced projections from the mPFC to the BLA in adult mice to measure axonal projections in vivo. Reconstruction of fibers in the BLA showed decreased density in cHets with comparable injection site density (FIGS. 4G, H), providing a substrate of altered axonal projections that may cause social deficits in cHets. To determine whether there are more widespread effects on layer V projecting circuits caused by Dyrk1a mutation, we investigated the corticospinal tract (CST), which contains the longest projections in the mammalian brain (39) and thus may be sensitive to alterations in growth. Using Cre-dependent reporter tdTomato in sagittal sections, we found that cHets exhibit significantly decreased density of CST projections, suggesting that multiple circuits dependent on layer V pyramidal neurons are altered in cHets (FIGS. 4I, J). Both decreased arborization of pyramidal neurons and decreased axonal projections in cortico-subcortical circuits provide a potential cellular link between alterations in brain growth and autism-relevant behavior.

### Example 4. Growth Signaling Pathways are Altered in Conditional Dyrk1a Mutants

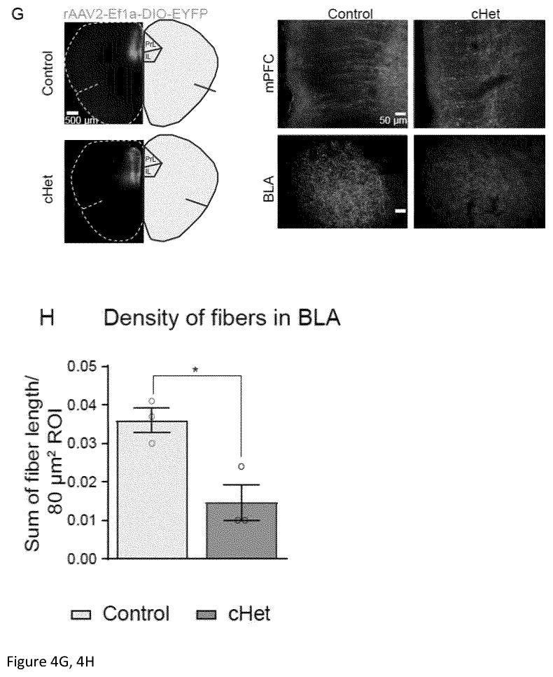

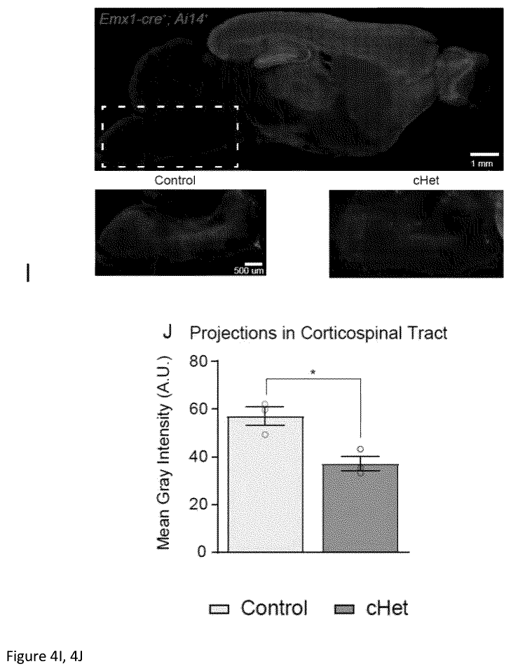

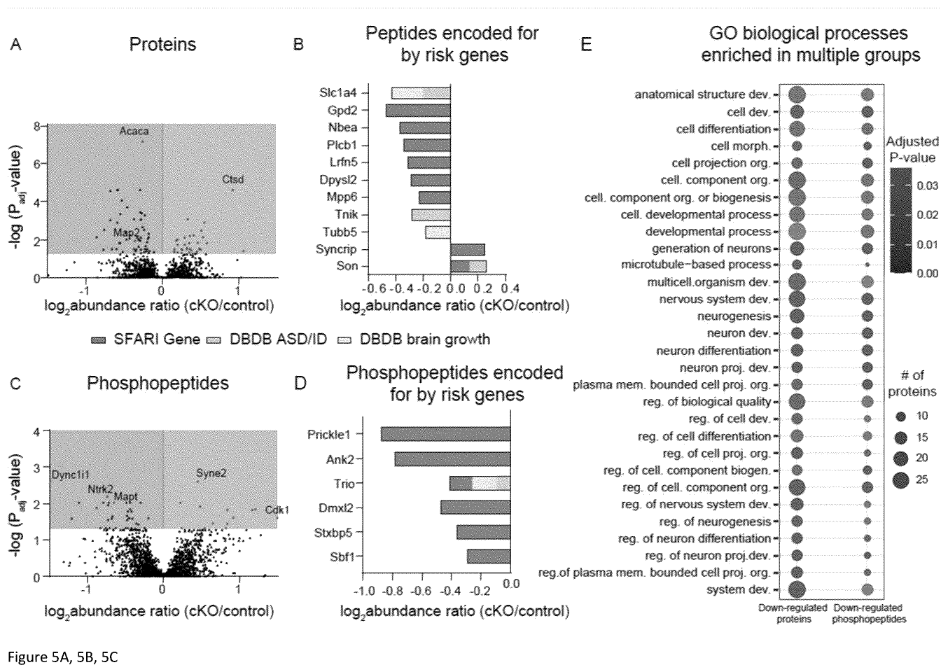

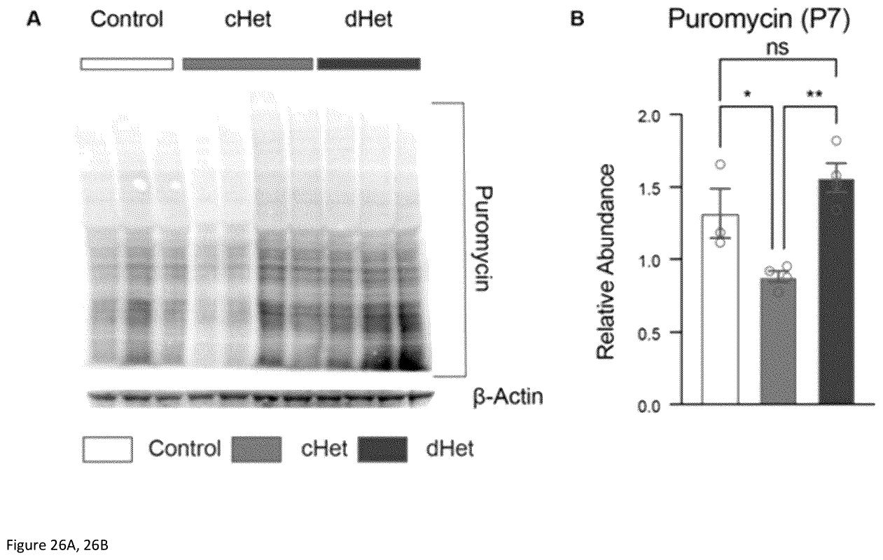

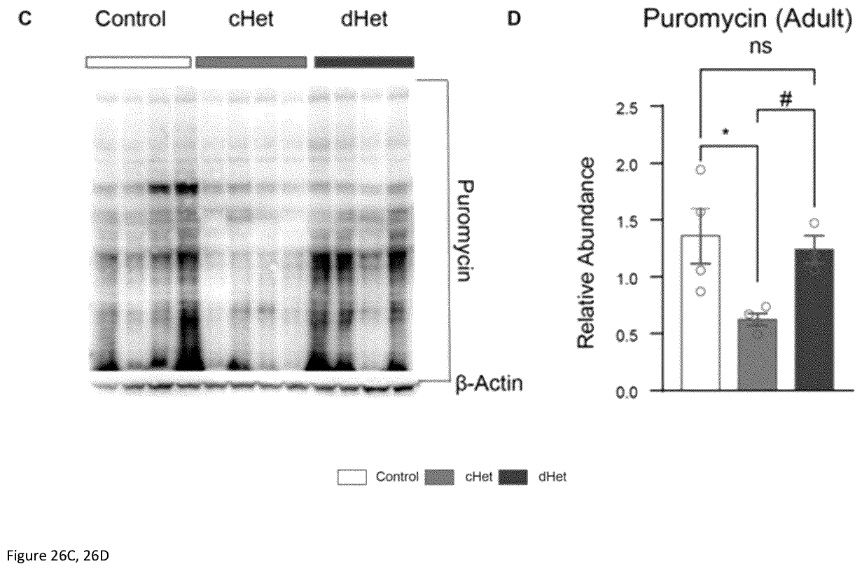

To investigate altered signaling cascades through which Dyrk1a mutations cause microcephaly, decreased neuronal growth, and ASD-relevant behaviors, we conducted a phospho-proteomic and proteomic screen via high-resolution tandem mass spectrometry coupled to liquid chromatography (LC-MS/MS). Our tandem mass tag (TMT) approach and phosphopeptide enrichment in P0 control and cKO cortices identified 89 significantly altered proteins with abnormal abundance in the cKO cortex at birth (56 decreased and 33 increased) (FIG. 5A, FIGS. 26A-26G). Ingenuity Pathway Analysis (IPA) identified axonal guidance signaling, RhoA signaling, and semaphorin signaling in neurons as significantly altered canonical pathways. IPA also identified BDNF, mTOR, and IGF-1 as predicted upstream regulators of the altered proteome. We then surveyed the altered proteome for proteins encoded by risk genes for ASD, ID, and brain growth abnormalities using SFARI gene and the Developmental Brain Database (DBDB), specifically using the categories “autism”, “intellectual disability”, “microcephaly”, “megalencephaly”, and “hemimegalencephaly”. We found that several proteins in the dataset are encoded for by ASD/ID risk genes or genes that impact brain growth (FIG. 5B).

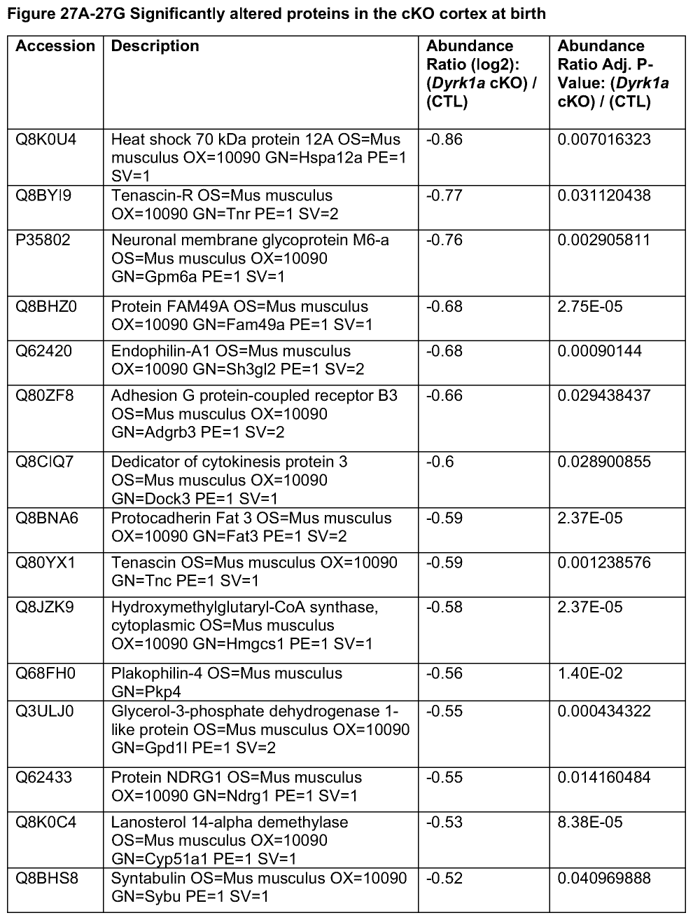

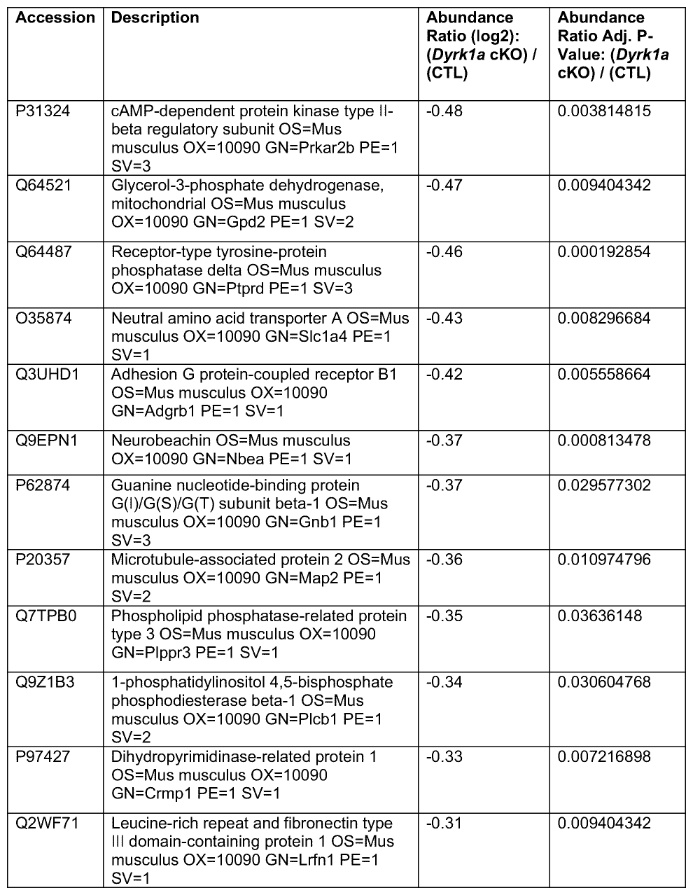

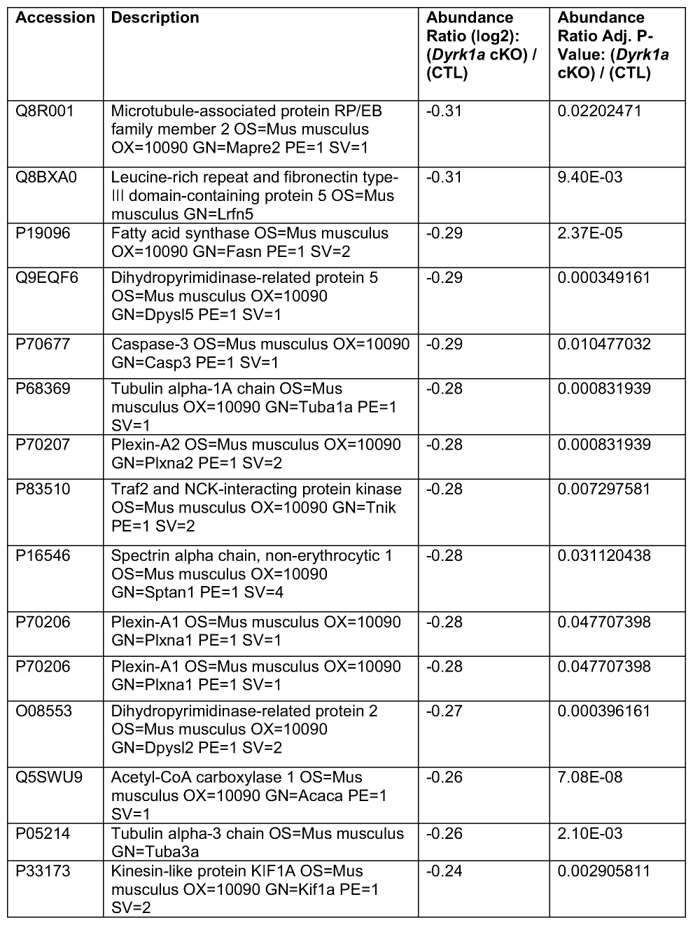

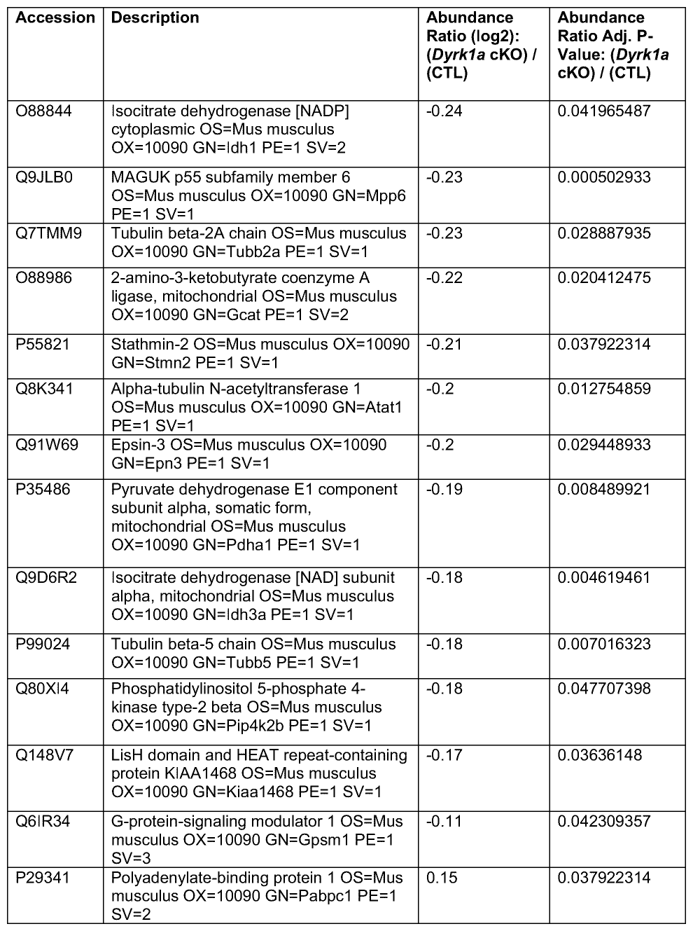

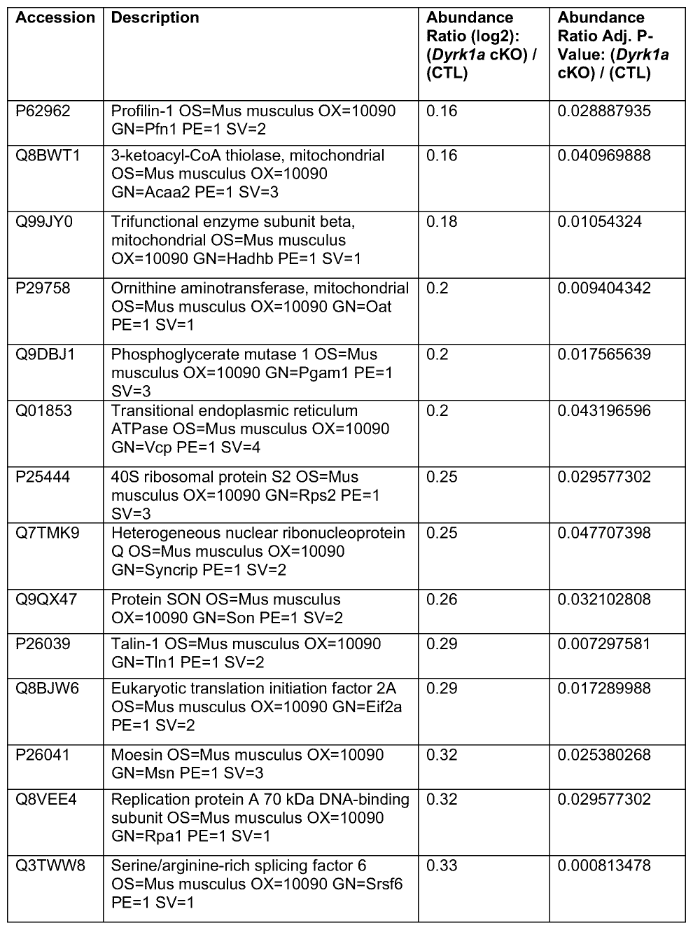

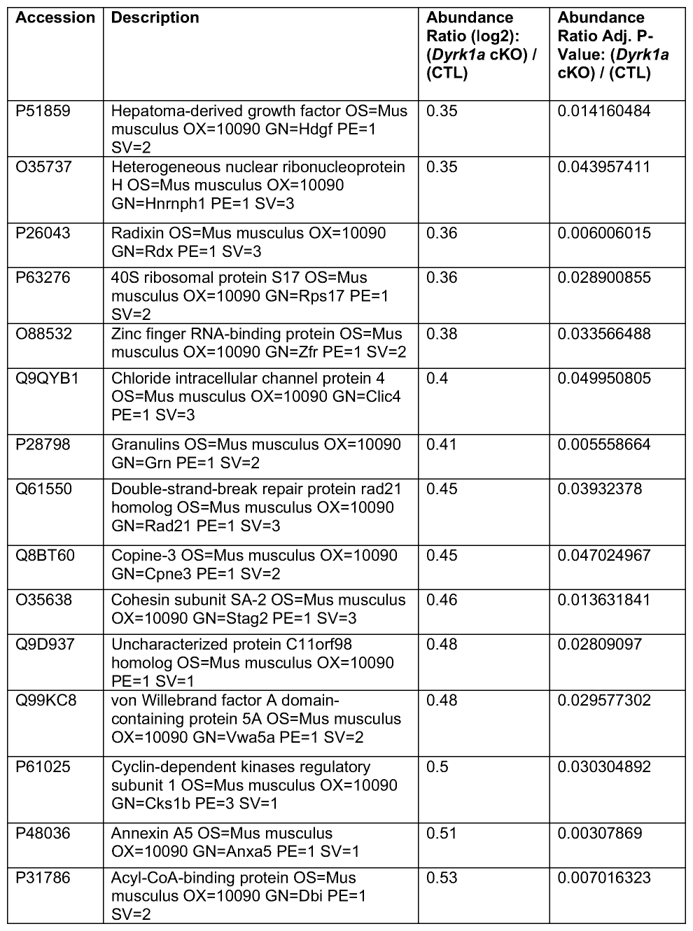

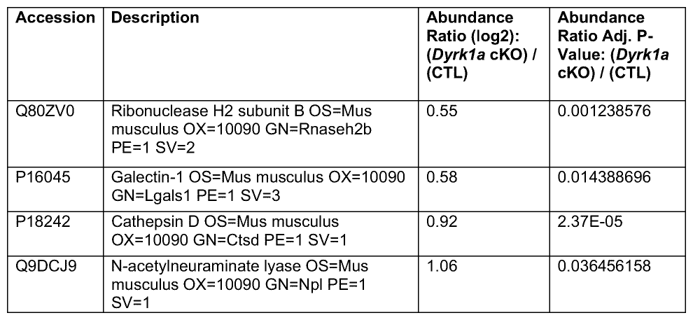

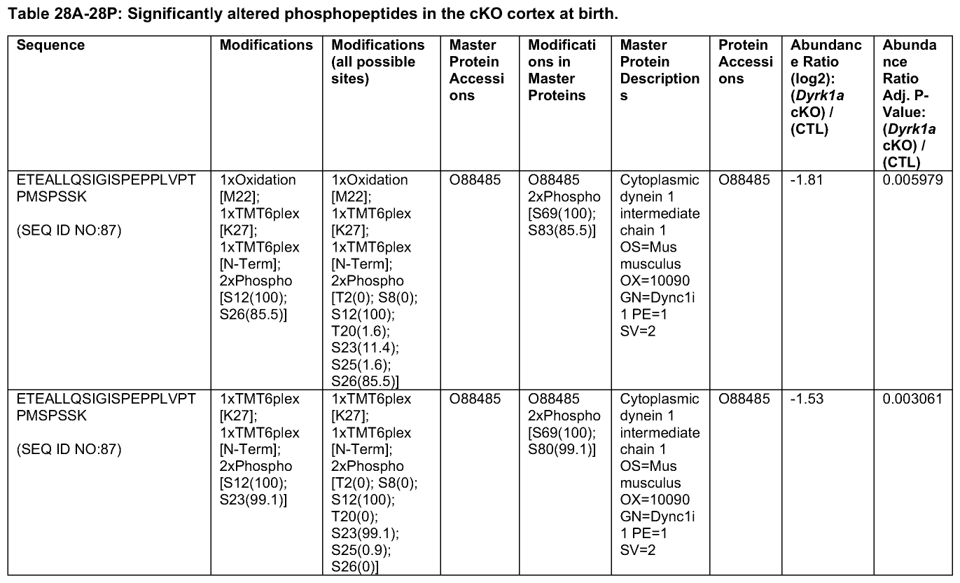

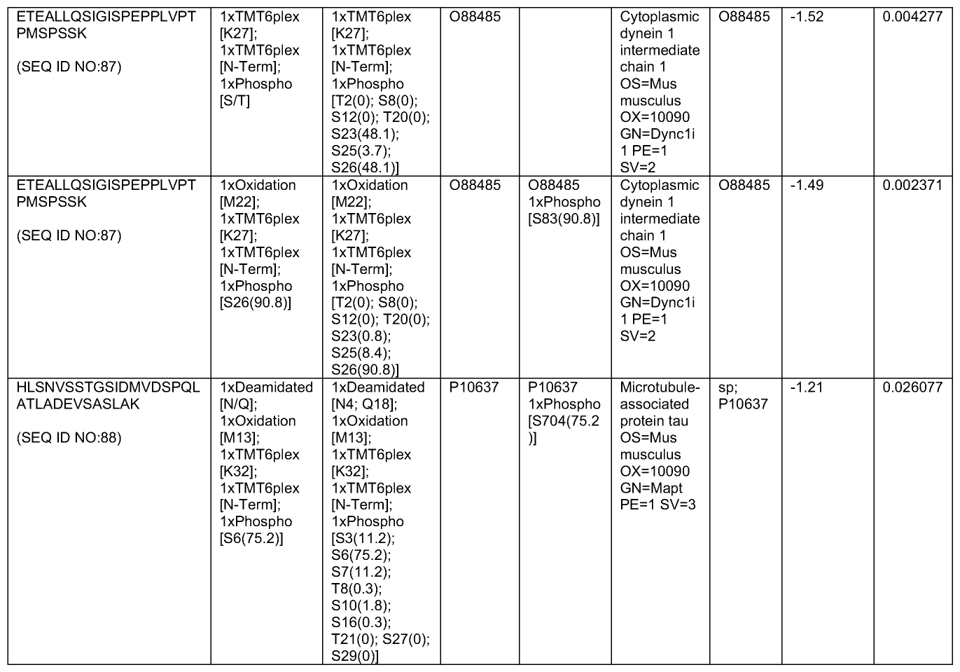

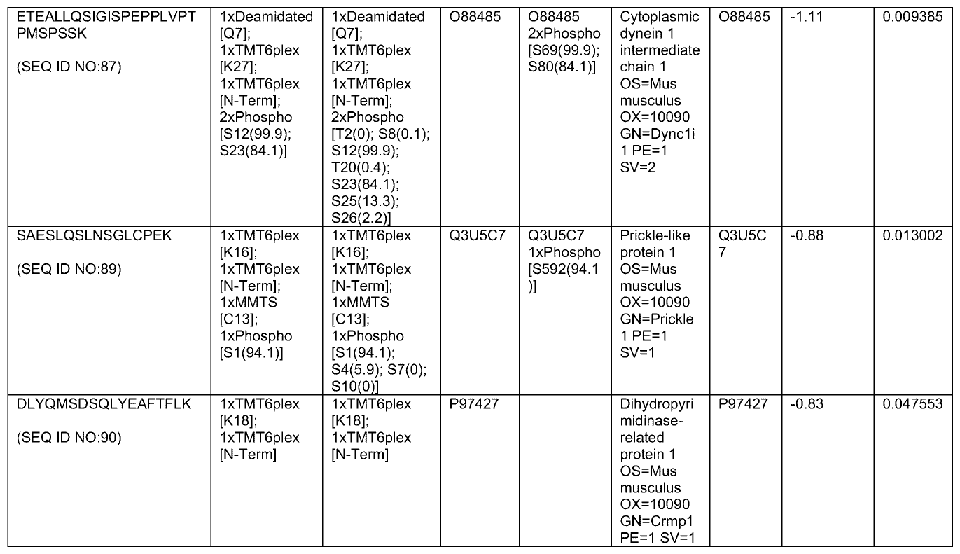

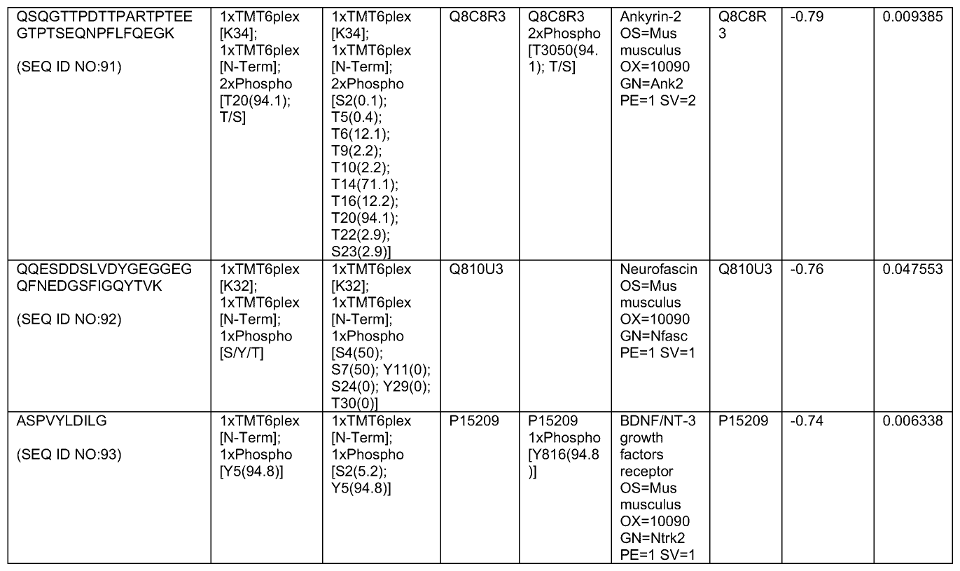

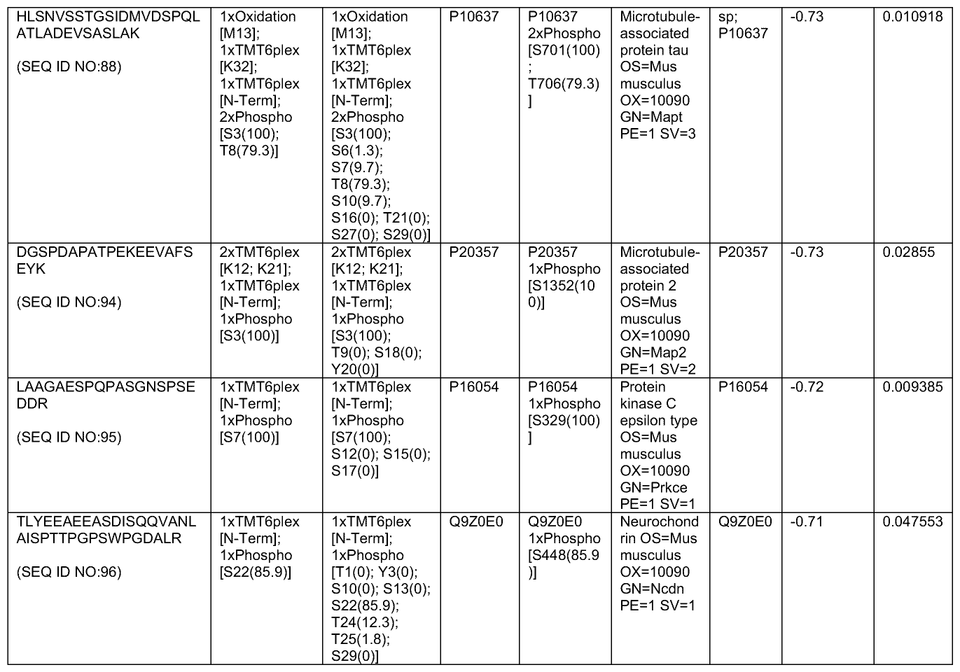

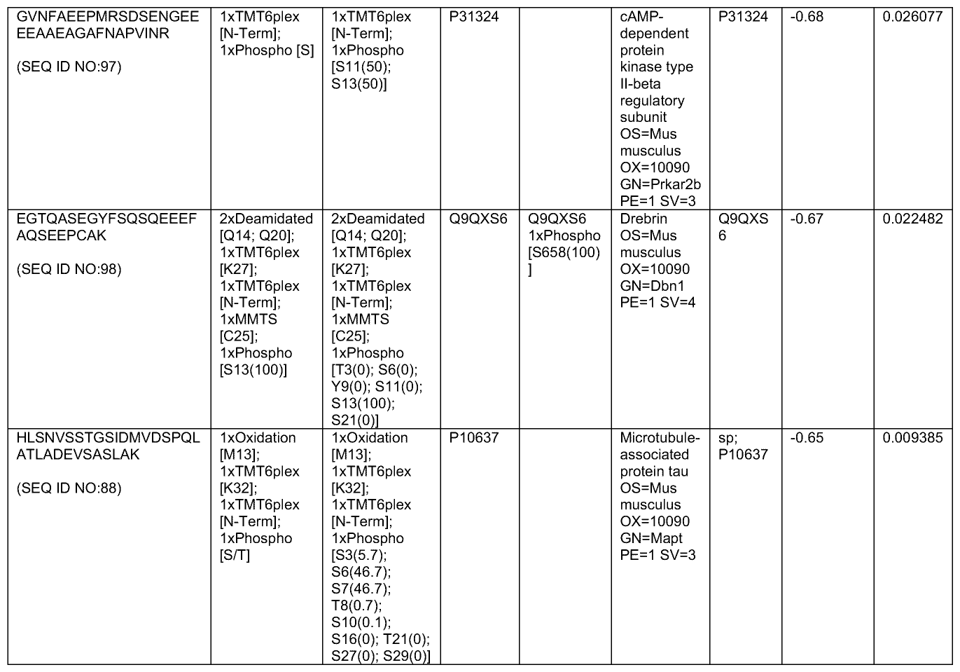

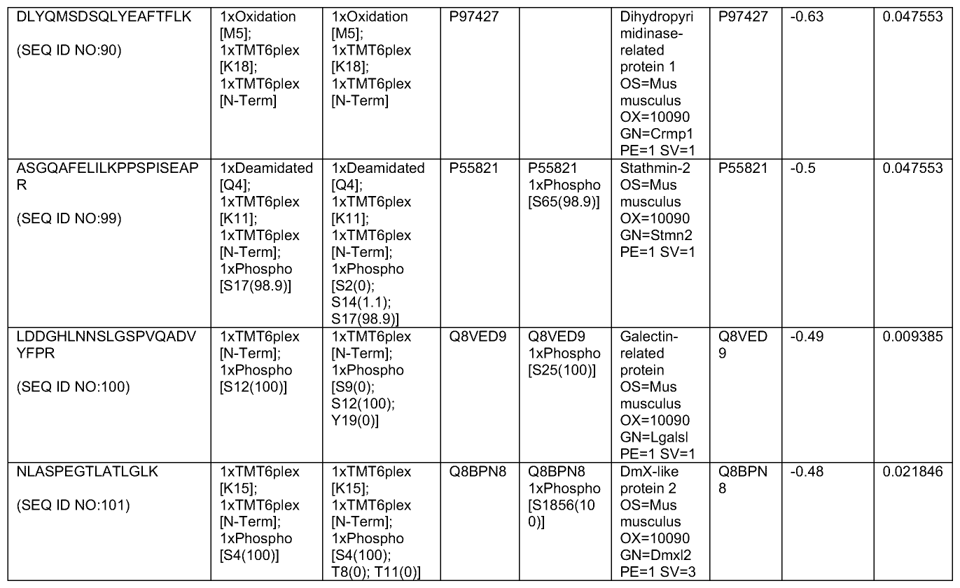

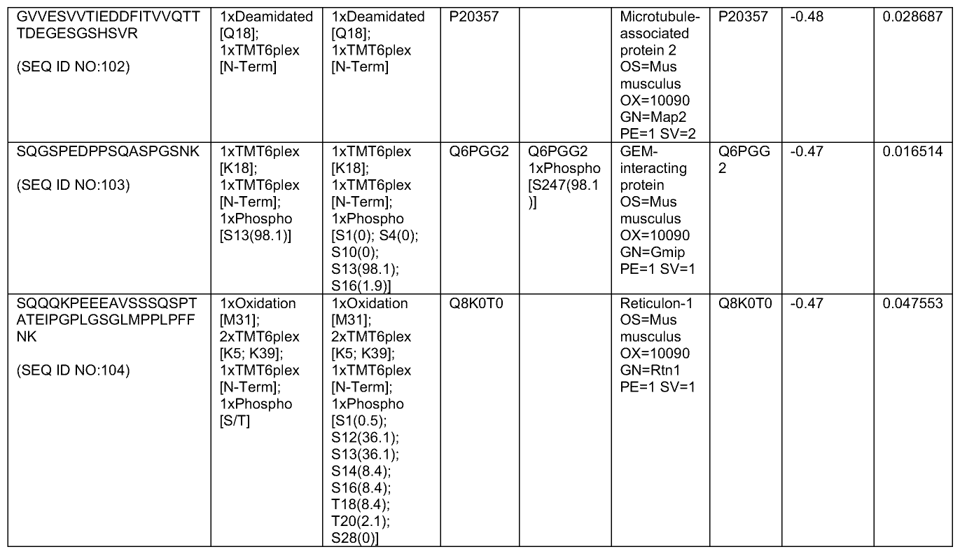

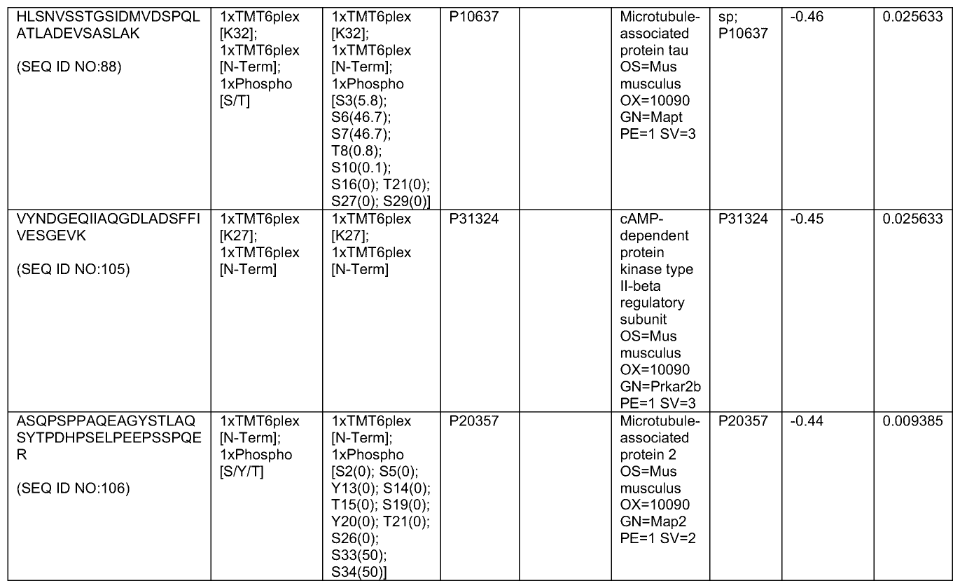

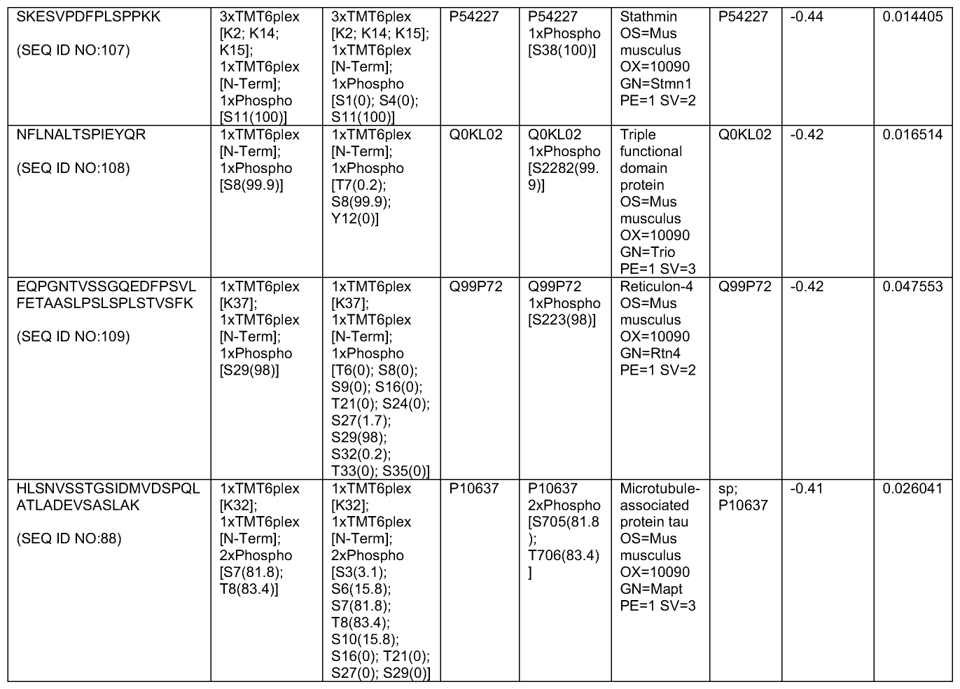

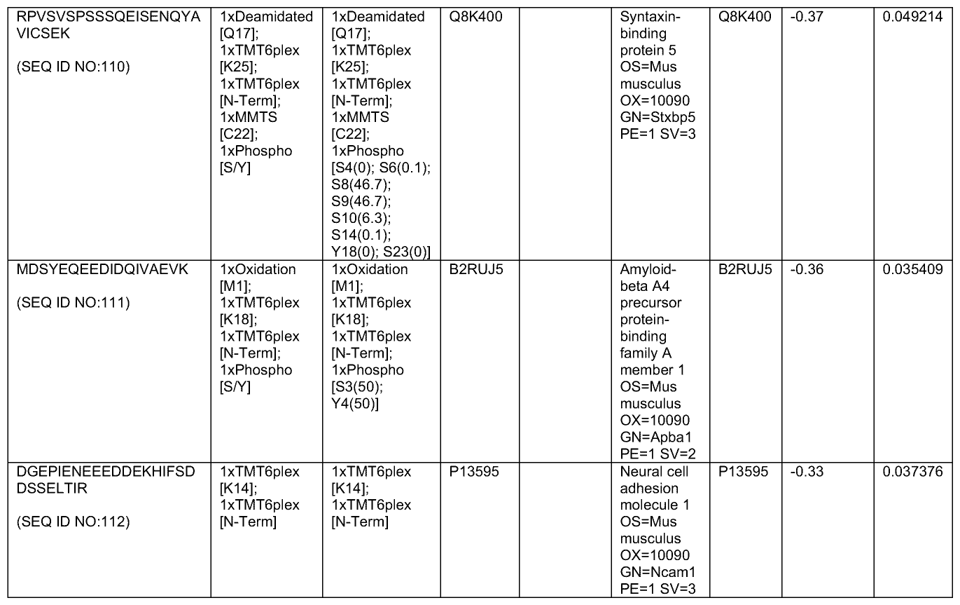

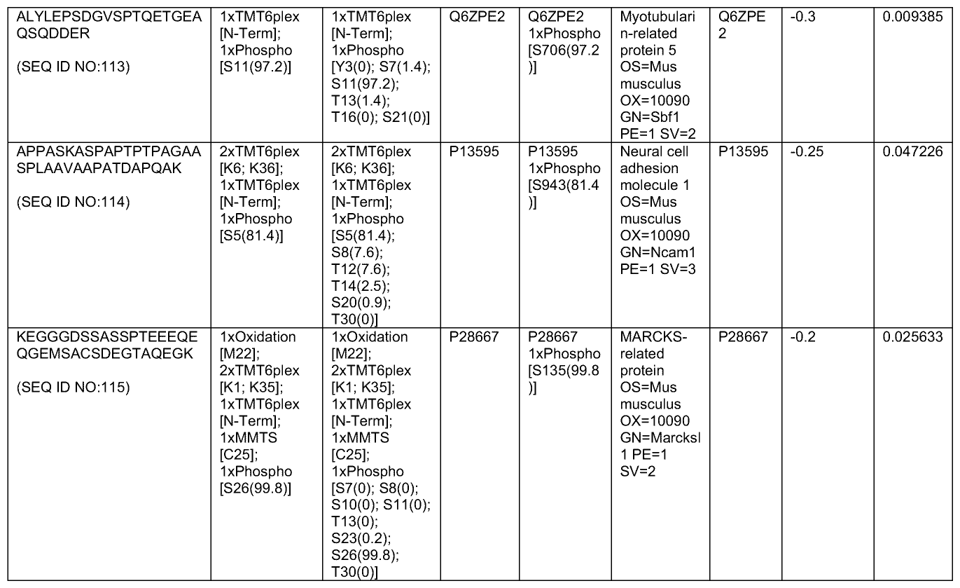

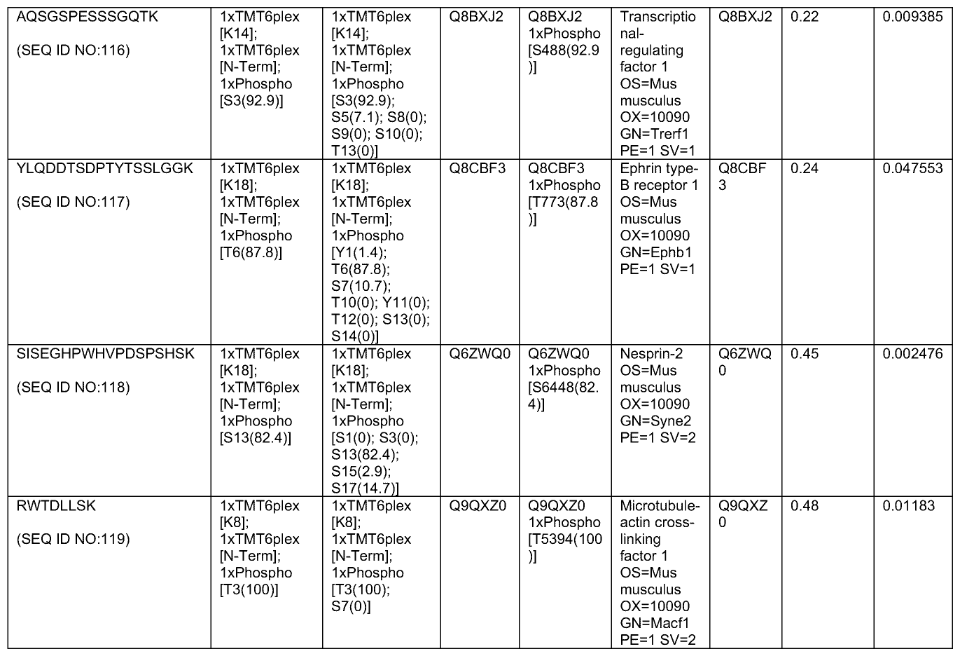

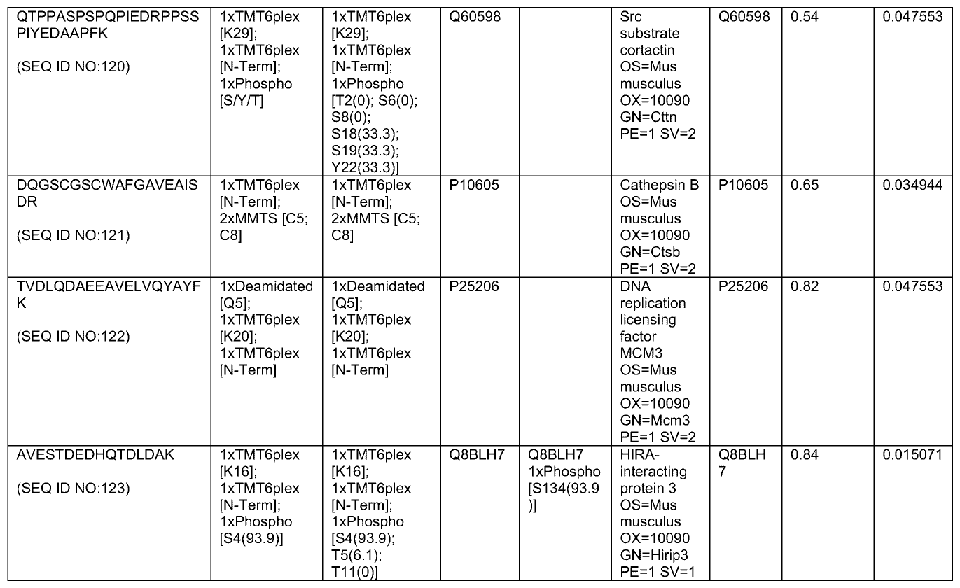

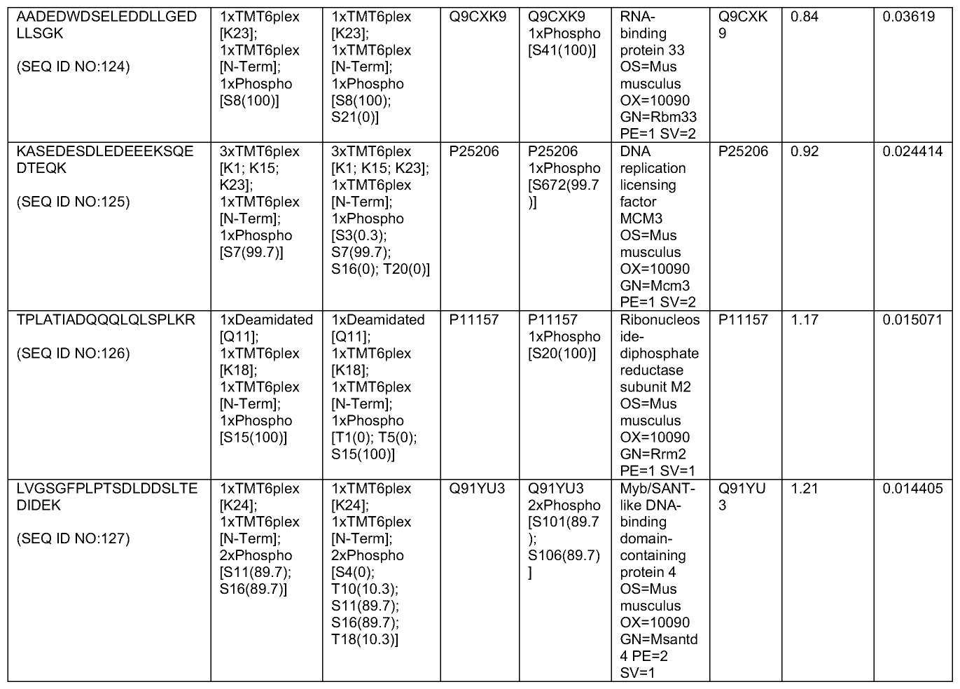

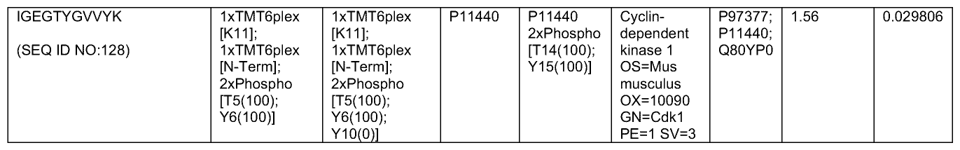

After phosphopeptide enrichment, 51 significantly altered phosphopeptide groups (35 phospho-proteins) were discovered (24 decreased and 11 increased) (FIG. 5C, FIGS. 27A-27P). IPA identified synaptogenesis signaling, axonal guidance, and ERK/MAPK as altered pathways. BDNF, ERK, and MAPK were found to be predicted upstream regulators. Using the same analysis against SFARI gene and DBDB, we identified multiple proteins in the altered phospho-proteome encoded for by ASD/ID/brain growth genes (FIG. 5D).

Gene ontology (GO) enrichment analysis revealed significant overlap shared between the down-regulated protein and down-regulated phosphopeptide groups, including GO terms “anatomical structure development” and “neuron projection development” (FIG. 5E). No biological processes were significantly enriched after correction in either the up-regulated phosphopeptide group or up-regulated protein group.

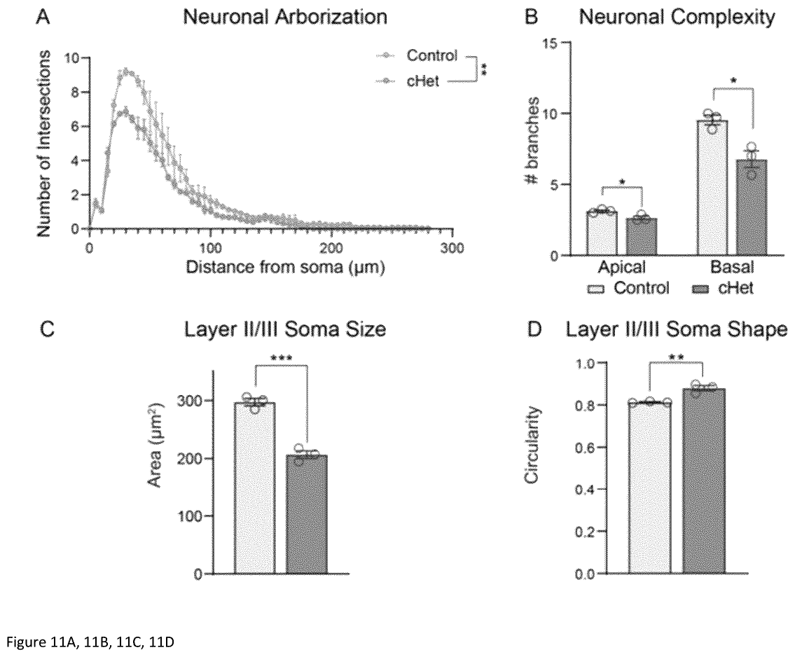

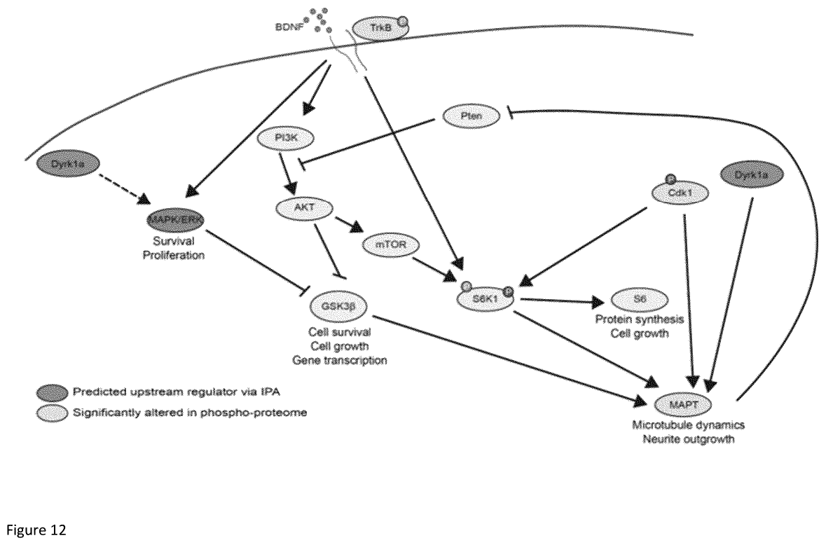

Noting BDNF as a predicted upstream regulator of both the altered proteome and phospho-proteome, we are interested to report significantly decreased phosphorylation of TrkB (Y816) in Dyrk1a cKOs. To validate this and other significantly altered targets in the phospho-proteome, we performed western blots on P0 controls and cKOs (FIGS. 5F, G). Indeed, p-TrkB (Y816) is decreased in Dyrk1a cKOs when normalized to total TrkB. We also validated increased inhibitory phosphorylation on Cdk1 relative to total Cdk1 and β-actin. Phosphorylation of MAPT/Tau was also identified as significantly decreased in the cKO dataset. Dyrk1a is known to phosphorylate Tau at Thr212, Ser202, and Ser404(40); however, the peptide identified by mass spectrometry in this experiment is phosphorylated at Ser704, which does not contain the Dyrk1a consensus sequence and thus may not be a direct target of Dyrk1a. We found that multiple isoforms of Tau are depleted in Dyrk1a cKOs. Map2 was found to be significantly decreased in the proteome, and indeed was found to be decreased in cKO cortical lysate (FIG. 5F, G). These data implicate signaling cascades that were previously not known to be altered by Dyrk1a mutations (FIG. 12

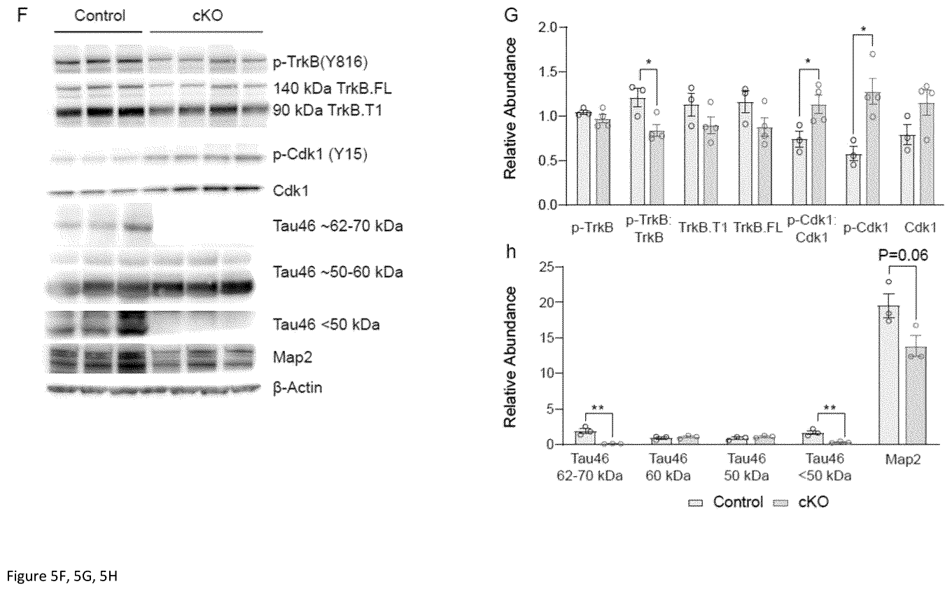

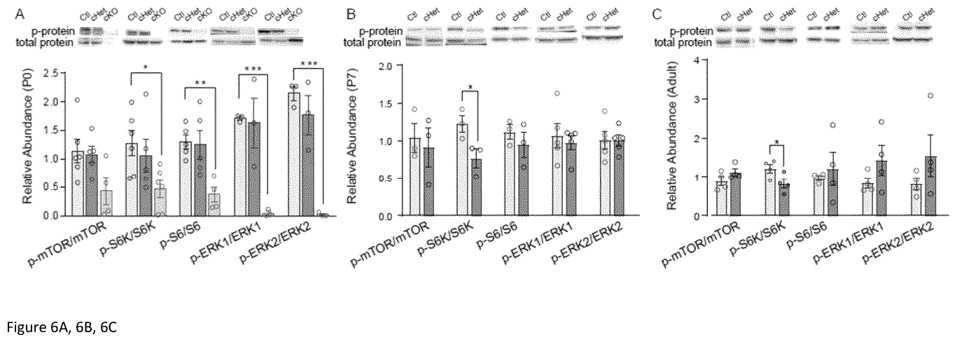

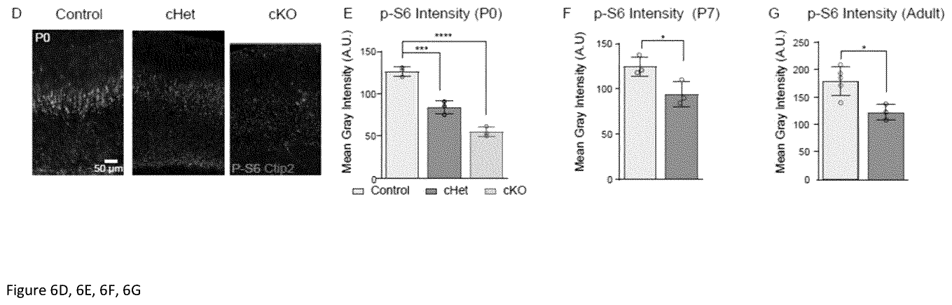

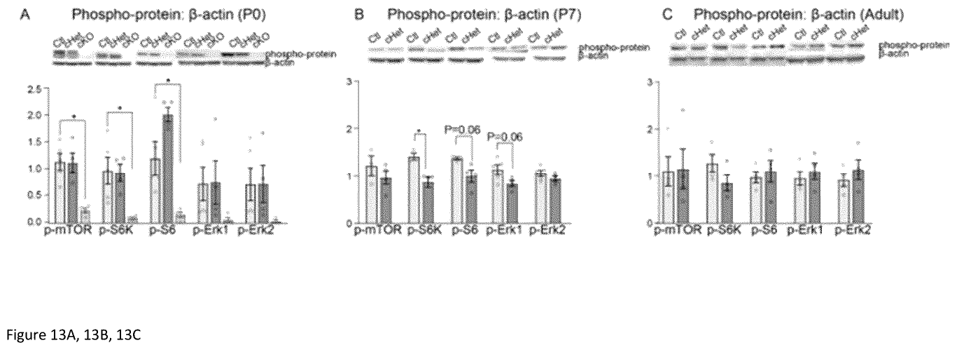

As mTOR was identified by IPA in Dyrk1a cKOs, and because mTOR is regulated by BDNF/TrkB signaling (41, 42), we next investigated whether activation of the mTOR signaling cascade is altered in cortical tissue. We performed western blots on cortical lysate at P0, P7, and adult timepoints. At P0, cHets showed no difference in the activation of mTOR signaling components; however, cKOs exhibit a severe reduction in phosphorylation of S6, S6K1, and ERK1/2 (FIGS. 6A, 13). This remarkably low activation of ERK1/2 aligns with the prediction by IPA that ERK/MAPK signaling is significantly disrupted in Dyrk1a cKOs and corroborates down-regulation of BDNF-TrkB signaling. Both P7 and adult cHets exhibit a significant decrease in phosphorylation of S6K1 (FIGS. 6B, C, 13). To determine whether there is a cell-selective effect of decreased mTOR signaling in Dyrk1a mutants, we used phosphorylation of S6 (Ser235/246), which is enriched in layer V pyramidal neurons (20), as a readout of mTORC1 activity. We found decreased p-S6 per putative layer V neuron (Ctip2+) in cHets at P0, P7, and adulthood (FIGS. 6D-G). In cKOs, p-S6 is virtually ablated in layer V neurons. We did note bright, fluorescent bodies that did not colocalize with DAPI or Ctip2. These have been noted in other mouse models of mTOR ablation (data not shown) and warrant further investigation (FIGS. 6D).

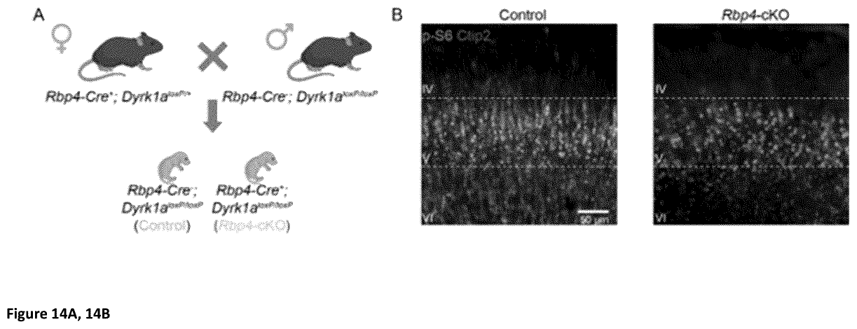

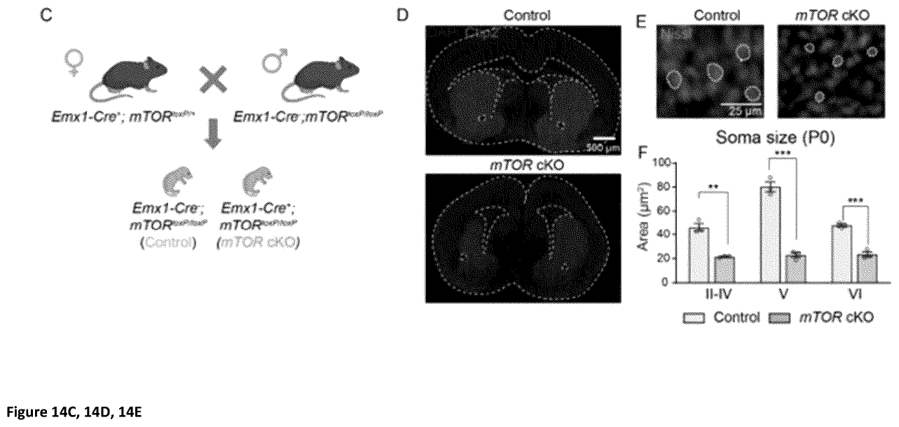

To test the necessity of mTOR for regulating cell soma size, we generated conditional mTOR cKOs (Emx1-cre⁺; mTOR^{loxP/loxP}) (FIGS. 14C, D). At P0, mTOR cKOs exhibit decreased soma size, comparable to Dyrk1a cKOs (FIGS. 14E, F). This phenocopy of Dyrk1a mutants by mTOR deletion suggests that decreased mTOR signaling may drive decreased cell size in Dyrk1a mutants. To determine whether decreased mTOR activation can be driven by deletion of Dyrk1a in layer V neurons, we measured p-S6 (Ser235/236) in P0 Rbp4-cKOs. Similar to Emx1-cKOs, P0 Rbp4-cKOs exhibit markedly decreased p-S6 intensity in layer V neurons (FIGS. 14A, B). These data provide a novel molecular mechanism by which Dyrk1a impacts growth signaling cascades, including mTOR and BDNF-TrkB, to regulate growth and connectivity.

### Example 5. Genetic Suppression of Pten or Pharmacological Treatment with IGF-1 Rescues Microcephaly and Neuronal Undergrowth in cHets

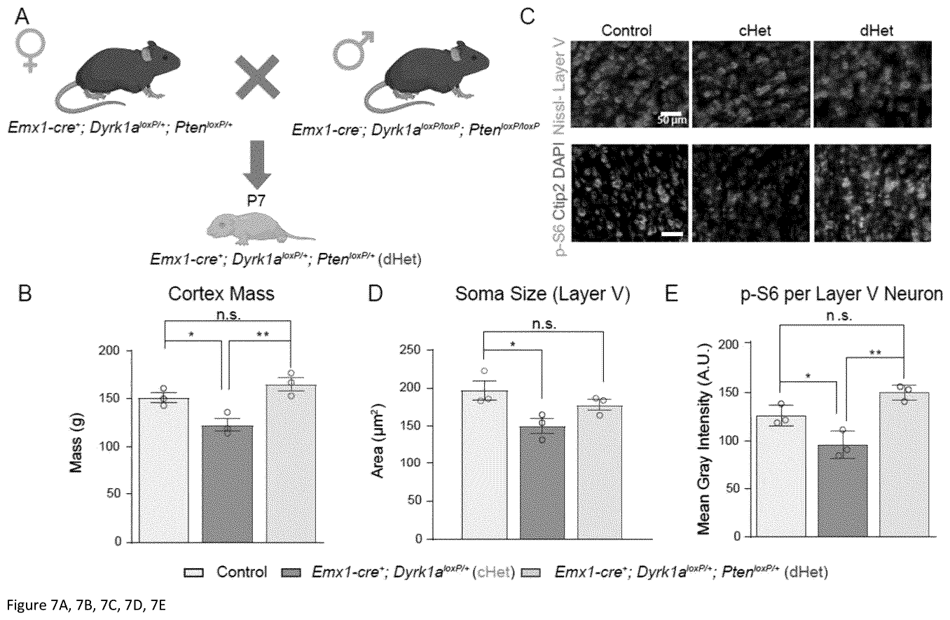

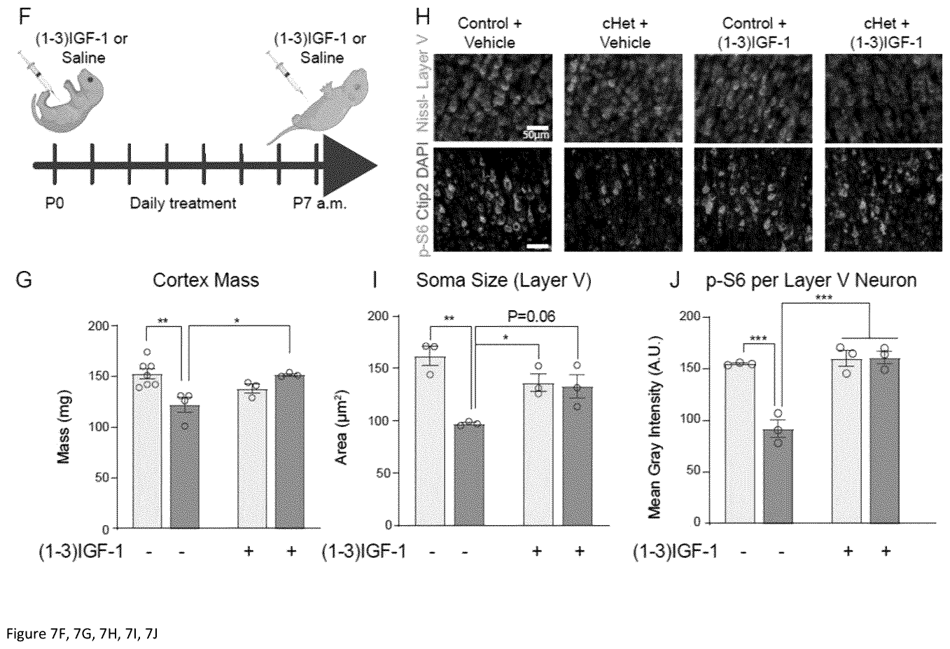

Given the decreased activation of BDNF-TrkB and mTOR signaling and the decrease in overall cortex mass and cellular size, we hypothesized that increasing mTOR signaling would rescue the observed deficits. Since Pten is a negative regulator of the PI3K/Akt/mTOR pathway (43), decreasing Pten dosage in cHets would remove the “brake” on mTOR signaling and be expected to rescue the observed microcephaly and decreased neuronal size. We generated conditional double heterozygous mutants (Emx1-cre⁺; Dyrk1a^{loxP/+}, Pten^{loxP/+}, “dHets”) to genetically suppress Pten in Dyrk1a cHets (FIG. 7A). At P7, the cortex mass of dHets is similar to controls (FIG. 7B). dHets also exhibit rescued soma size of layer V neurons, and p-S6 per layer V neuron is significantly higher than cHets (FIGS. 7C-E). Thus, genetic suppression of Pten in Dyrk1a mutants rescues the decreased cortical mass and cellular size through increased mTOR signaling. Moreover, the data suggest epistatic regulation between Pten and Dyrk1a whereby Pten must be present at normal levels to observe the effects of Dyrk1a mutations.

We next aimed to increase mTOR signaling via pharmacological intervention. Because activation of TrkB receptor is decreased in cKOs, treating mice with exogenous BDNF may not be effective. To circumvent this issue while stimulating the same signaling cascades, we injected neonatal pups with (1-3)IGF-1 (GPE), a tripeptide cleaved from the N-terminus of IGF-1 (44). Pups were injected daily from P0 to P7 with 10 μg/g (1-3)IGF-1 or vehicle based on a previously described treatment paradigm (45). Eight hours post-injection on P7, pups were sacrificed, and brains were fixed (FIG. 7F). Dyrk1a cHets treated with (1-3)IGF-1 exhibit cortex mass similar to vehicle-treated controls, and (1-3)IGF-1 treatment had no effect on cortex mass of controls (FIG. 7G). (1-3)IGF-1 treated Dyrk1a cHets also display soma size comparable to controls as well as normal levels of p-S6 per layer V neuron (FIGS. 7H-J). Taken together, these data show that neonatal (1-3)IGF-1 treatment in Dyrk1a cHets rescues cortical and neuronal undergrowth corresponding with increased p-S6 levels.

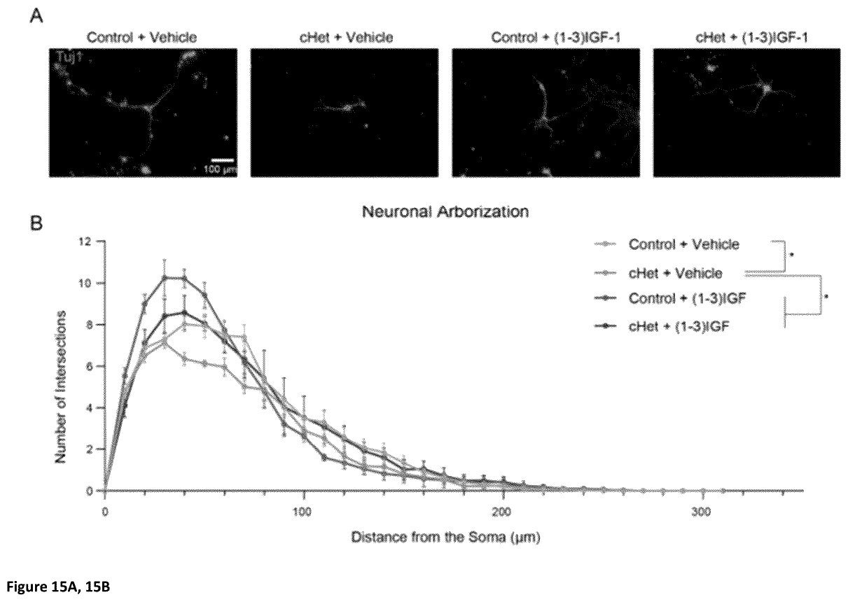

Lastly, to test whether (1-3)IGF-1 treatment rescues the decreased complexity of Dyrk1a cHet neurons, we cultured control and cHet primary cortical neurons. Neurons analyzed were restricted to Ctip2+ neurons, as we are focused on the impact of Dyrk1a mutations on the growth and connectivity of layer V pyramidal neurons. On DIV15, vehicle-treated cHet neurons displayed significantly decreased complexity compared to controls. This was rescued by treatment with 100 ng/mL (1-3)IGF-1 daily from DIV13 to DIV15 while (1-3)IGF-1 did not affect control neurons (FIG. 15). These data show that (1-3)IGF-1 treatment rescues the decreased neuronal complexity in vitro in Dyrk1a cHets.

### Example 6. Methods and Materials for Examples 1-5

All mice used in the study were obtained from the Jackson Laboratory or MMRC and have been previously described, including C57BL/6-Dyrk1atmI/J (Dyrk1a^{loxP/loxP}, stock #027801), Gt (ROSA)26Sor^{tm14 (CAG-tdTomato)Hze}/J (Ai14 or tdTomato, stock #007914), Pten^{tm1Hwu} (Pten^{loxP/loxP}, stock #006440), Emx1^{tm1 (cre)Krj} (Emx1-cre^{+/-}, stock #005628), and B6.FVB (Cg)-Tg (Rbp4-cre)KL 100Gsat/Mmucd (Rbp4-cre^{+/-}, stock #037128-UCD). WT and Emx1-cre⁻; Dyrk1a^{loxP/+} mice were used as controls. To avoid germline recombination in males, Emx1-cre⁻; Dyrk1a^{loxP/loxP} P males were bred with Emx1-cre⁺; Dyrk1a^{loxP/+} females. The same breeding strategy was used for experiments involving Rbp4-cre, mTOR^{loxP/loxP}, and Pten^{loxP/+}, Genomic DNA isolated from ear samples was used for PCR to confirm genotypes. All animal experiments were conducted in accordance with National Institutes of Health and Association for Assessment and Accreditation of Laboratory Animal Care guidelines and were approved by The Scripps Research Institute's Institutional Animal Care and Use Committee. Mixed sexes were used for all cellular and molecular assays. Male mice were used for behavioral experiments. Mixed sexes of even numbers were used for proteomic analysis, and females were used for the phospho-enrichment and analysis.

Mouse behavior tests: Three-Chamber Social Approach Test: Male mice were tested as previously described (1, 2). Briefly, after at least 2 weeks housing under reversed light cycle, each mouse was habituated in the three-chamber box for 5 min. before a stimulus mouse was placed into a tube in the “social” chamber. The stimulus mice used were young adult male mice. The time spent in each chamber (“mouse+tube”, “empty tube”, “center”) was automatically scored using Ethovision XT software (Noldus).

Marble Burying: Male mice were placed individually in a cage with 5 cm of ¼″ corncob bedding and 20 black marbles (14.3 mm in diameter) arranged in a 4×5 matrix and left undisturbed for 30 min. The number of marbles that were at least two-thirds buried at the end of the trial were counted.

Tail Suspension Test: Male mice were suspended from a hook with medical tape attached ˜2 cm from the tail tip for 6 min. The Observer software (Noldus) was used to code and analyze the amount of time that mice were immobile during the trial.

Open Field Test: Male mice were placed individually in the center of the open field arena (43.8×43.8×32.8 cm) under white light with 70 dB background white noise for 5 min. Total distance moved, velocity, and time spent in the center versus corners/sides (thigmotaxis) were recorded automatically using Ethovision XT software.

Acoustic Startle and Pre pulse Inhibition: Each mouse was placed inside a clear acrylic tube (5 cm in diameter, 10 cm long) secured to a platform with a piezoelectric accelerometer attached beneath the tube (San Diego Instruments) inside a ventilated, sound-attenuating chamber. Following a 5 min. acclimation period, mice receive trials of white noise stimuli in three phases: I) baseline startle, II) mixed startle and pre-pulse inhibition, and III) final startle. During the test, trials are presented at variable 8-23 s inter-trial intervals, and 70 dB background white noise is present throughout. Phases I and III consist of 6 startle trials with a white noise stimulus of 40 ms at 120 dB. During phase II, mice receive a total of 52 trials of three types, presented in a pseudorandom order: i) 12 startle trials (as in phases I and III), ii) 10 control trials (no stimulus), iii) pre-pulse inhibition trials (20 ms pre-pulse stimulus at 4, 8, or 16 dB above background, followed by 120 dB startle stimulus 100 ms after pre-pulse onset), 10 for each pre-pulse stimulus. The maximum whole-body flinch response to each startle pulse (whether preceded by a pre-pulse or not) are recorded using SR-Lab software (San Diego Instruments), which takes 65 consecutive 1 ms readings from the beginning of stimulus onset. Response to each type of stimulus was averaged across presentations within each phase. Percent pre-pulse inhibition was calculated as [(phase II startle trial−pre-pulse trial)/(phase II startle trial)×100].

(1-3)IGF-1 treatment: (1-3)IGF-1 was acquired from VWR/Bachem (catalog #H-2468.1000) and dissolved in saline with 0.01% BSA fresh every day. Intraperitoneal injections of 10 μg/g (1-3)IGF-1 or vehicle were delivered daily at 8 a.m. starting on P1 and ending on P7. This dosage was chosen based on multiple studies of (1-3)IGF-1 treatment in ASD mouse models that showed brain penetrance and increased signaling downstream of IGF-1 within 3 hours post-injection (3, 4). Pups were individually removed, injected, and placed back in the home cage with the dam. On P7, 6 hours post-injection, pups were rapidly decapitated for downstream analysis.

Isotropic Fractionator: Isotropic fractionator was used as previously described (5). Briefly, adult animals were perfused with 4% PFA, and pups underwent rapid decapitation and dissection. Brains were fixed in 4% PFA for one week, and then the cortex was dissected out and dissociated in a 7 mL glass tissue homogenizer (Kontes Glass) in dissociation buffer (1% Triton X-100 in 40 mM sodium citrate). 1 mL of nuclei was removed and stained with 1:50 DAPI (Invitrogen, catalog #D3571) before being loaded onto a hemocytometer (Fisher Scientific, catalog #0267110). Per animal, 6 technical replicates of 104, nuclei were imaged and counted in the hemocytometer (0.1 μL nuclei counted per technical replicate). After calculating the total cell number per cortex, the number of cells was divided by mg of cortex to obtain density.

Flow cytometry: After isotropic fractionator, nuclei were stained with DAPI (1:100) and primary (anti-NeuN antibody (1:500; 104225, Abcam)) and secondary (Goat anti-Mouse 488 (1:2000, A21121, Life Technologies)) antibodies, then filtered through a 30 μm diameter cell strainer (#352235, Falcon) before analysis. On a Gallios flow cytometer analyzer (Beckman Coulter), DAPI fluorescence was collected with a 450/50 filter (405 nm laser) and Alexa 488 with a 550 SP filter (488 nm laser). Background fluorescence was determined using a control with only the secondary antibody. The Neu-N+ population was determined by gating the population of single, DAPI+ events above background using FlowJo software. Total neuronal and non-neuronal numbers were calculated by taking the percentage (as determined by flow cytometry) of the total cortical cell number from the same biological sample.

Immunohistochemistry: Adult animals were perfused with 4% PFA, and pups underwent rapid decapitation and dissection. After 18 hours post-fixation in 4% PFA, brains were incubated in a 20% sucrose/PBS solution at 4° C. for 2-3 days and embedded in Tissue-Tek OCT compound (Sakura). 40 μm sections were collected on Superfrost/Plus slides and stained. The following primary antibodies were used following antigen retrieval (10 min. in boiling 10× citrate buffer): anti-cleaved caspase 3 (1:1000, Cell Signaling Technology, catalog #9661S), anti-Ctip2 (1:2000, Abcam, catalog #18465), anti-p-S6 Ser235/236 (1:2000, Cell Signaling Technology, catalog #4858S). AlexaFluor-488, -594, and -647-conjugated secondary antibodies (1:2000) were purchased from Invitrogen. DAPI was used for nuclear labeling (Invitrogen, catalog #D3571). Slides were mounted with Vectashield HardSet and images were obtained with an Olympus VS120 microscope and processed using the VS-DESKTOP software (Olympus) or ImageJ (NIH).

For morphological measurements (cortical thickness), fixed lines were drawn horizontally at the bottom of a coronal section and a perpendicular vertical line through the midline. Lines at varying degrees were then drawn from the bottom landmark, and cortical thickness was measured at each measurement angle, agnostic of the shape or size of the brain section. This was conducted across 2-3 plane-matched sections per animal. Ventricle size was analyzed by drawing a region of interest (ROI) around the entire ventricle in 2-3 plane-matched sections per animal.

For analysis of laminar marker counts and density, ROIs were cropped in the somatosensory cortex and normalized for width, with the height of the ROI beginning at the top of the ventricle and ending at the pial surface. In ImageJ, the ROI was divided into 10 equal bins. Ctip2+ neurons were counted in each bin using the Cell Counter tool in ImageJ, then DAPI+ cells were counted in each bin. The density of putative layer V neurons was measured by dividing Ctip2+ neurons by DAPI+ cells per bin.

To measure apoptosis in the cortex, P0 and P4 sections were stained with cleaved caspase 3 (CC3). One whole cortical hemisphere was defined as an ROI; and for each animal, counts of cells positive for CC3 were obtained from 3 plane-matched sections per animal.

For p-S6 intensity, P0, P7, and adult sections were stained with Ctip2 and p-S6 (Ser235/236). This specific antibody has been shown to detect p-S6 in vivo in mice (6, 7). After image acquisition, Ctip2+ neurons were measured for p-S6 mean gray intensity. 25-30 neurons were measured per section and 2 plane-matched sections were analyzed per animal.

For soma size analysis, sections were stained with NeuroTrace green fluorescent Nissl stain (1:100 dilution, Life Technologies catalog #N-21480) and washed with PBS. After image acquisition, somas in each layer in somatosensory cortex (51) were measured using the ImageJ freehand tool. Layers were denoted based on anatomical boundaries using DAPI staining. 30-35 somas were measured per layer and 2-3 plane-matched sections were analyzed per animal. This was done based on a previously published technique (8).

Anterograde tracing: rAAV2-Ef1a-DIO-ChR2 (H134R)-EYFP was obtained from University of North Carolina Vector Core. For injections, mice were anaesthetized under isoflurane and placed onto a stereotaxic frame. Bregma and lambda were used to find the coordinates of the mPFC (1.8 mm anterior to bregma, 0.3 mm lateral to midline and 2.2 mm ventral to bregma). The virus was injected by a syringe pump at a rate of 50 nL min′, with 100 nL volume injected per site in each mouse. Mice were perfused 2 weeks later, allowing for the expression of EYFP. 50 μm-thick coronal brain sections were collected to image mPFC and axon terminals. For analysis, images were acquired, and fibers were reconstructed using the Simple Neurite Tracer plugin on ImageJ. Number of reconstructed fibers was measured relative to the 80 μm² ROI.

TMT labelling, phosphopeptide enrichment and quantitative mass spectrometry-based proteomics: Cortices were isolated from P0 control (N=6) and cKO pups (N=7) and dissociated using a dounce homogenizer in 1× lysis buffer (Cell Signaling Technologies). Protein was determined using the BCA protein assay (9) from Pierce as per the manufacturer's instructions. Two 100 μg aliquots (total 200 μg protein per sample) were dried under vacuum and processed for digestion using two independent micro S-Traps™ (Protifi, Huntington, NY) according to the manufacturer's instructions. Briefly, the dried protein was resuspended in 224, of 5% SDS, reduced using DTT at 55° C. for 10 min., alkylated using methyl methanethiosulfonate (MMTS) at room temperature for 10 min. and then spun down at 20K for 10 min. Subsequently, 2.54, of phosphoric acid was added to the sample, followed by 1654, of a mixture of HPLC grade methanol and Protifi binding/wash buffer. Following loading onto the S-Trap™, samples were washed three times using centrifugation and trypsin was added in 50 mM TEAB at a 1:25 w/w ratio. The S-Trap™ column was incubated for 1 hour at 47° C. Following this incubation, 404, of 50 mM triethylammonium bicarbonate (TEAB) was added to the 5-Trap™ and the peptides were eluted using centrifugation. Elution was repeated once. A third elution using 3511L of 50% acetonitrile (ACN) was also performed and the eluted peptides were dried under vacuum. Peptides were reconstituted in 50 mM TEAB, and their concentrations were determined using the Pierce™ quantitative fluorometric peptide assay (Thermo Fisher Scientific, Waltham, MA). Seventy micrograms of peptides were labelled with TMT labels (10-plex) according to the manufacturer's instructions (Thermo Fisher Scientific, Waltham, MA) and pooled. The pooled plexed samples (630 or 700 μg total) were dried under vacuum, resolubilized in 1% TFA, desalted using OASIS HLB 1 cc solid phase extraction cartridges (Waters, Milldford, MA) and then dried once again using vacuum. TMT-labeled phosphopeptides for mass spectrometry were enriched using the High-Select™ Fe-NTA Phosphopeptide Enrichment Kit from Thermo Fisher Scientific according to the manufacturer's instructions. The TMT-labelled non-phosphopeptide complement was cleaned up for mass spectrometry using a C18 ZipTip according to the manufacturer's instructions (Millipore, Billerica, MA). For mass spectrometry, dried TMT-labelled peptides (phosphopeptides and non-phosphopeptides) were reconstituted in 54, of 0.1% formic acid, and mass spectrometry was performed as described previously with the exception that separation was performed on as EASY PepMap™ RSLC C18 column (2 μm, 100 Å, 75 μm×50 cm, Thermo Scientific, San Jose, CA). Ions were created with an EASY Spray source (Thermo Scientific, San Jose, CA) held at 50° C. using a voltage of 1.9 kV (10).

Proteomic data processing and statistical analysis: Quantitative analysis of the TMT experiments was performed simultaneously to protein identification using Proteome Discoverer 2.3 software. The precursor and fragment ion mass tolerances were set to lOppm, 0.6 Da, respectively), enzyme was Trypsin with a maximum of 2 missed cleavages and Uniprot Mouse proteome FASTA file was used in SEQUEST searches. The impurity correction factors obtained from Thermo Fisher Scientific for each kit was included in the search and quantification. The following settings were used to search the phospho-enriched data; dynamic modifications; Oxidation/+15.995 Da (M), Deamidated/+0.984 Da (N, Q), Phospho/+79.966 Da (S, T, Y) and static modifications of TMT6plex/+229.163 Da (N-Terminus, K), MIVITS+45.987 (C). Only unique+ Razor peptides were considered for quantification purposes. Percolator feature of Proteome Discoverer 2.3 was used to set a false discovery rate (FDR) of 0.01. IMP-ptmRS node was used to calculate probability values for each putative phosphorylation site (11). Only phosphopeptides with a probability value >75% were included. Total Peptide Abundance normalization method was used to adjust for loading bias and Protein Abundance Based method was used to calculate the protein level ratios. Co-isolation threshold and SPS Mass Matches threshold were set to 50 and 65, respectively. The non-enriched dataset was analyzed in the same fashion except for omission phosphorylation in SEQUEST search and IMP-ptmRS node in PD workflow. Individual proteins were used to compare treatment groups to controls using ANOVA option in Proteome Discoverer and both P values and adjusted P values were generated.

For predicted upstream analysis, IPA Ingenuity Upstream Regulator was used to identify predicted upstream transcriptional regulators that may account for the altered protein expression in the given dataset. This was measured using an overlap P value and an activation z-score based on the altered proteins and phospho-proteins. GO terms were generated using geneontology.org, and the dot plot was generated using a previously described R script (12).

Western blot analysis: Following rapid decapitation, cortices were dissected and homogenized in cell lysis buffer (CST) containing the Protease Inhibitor Cocktail (Roche) and phosphatase inhibitors (Sigma-Aldrich) using a dounce homogenizer. Total protein concentrations were measured using the Pierce BCA Protein Assay Kit (ThermoScientific). 50 μg of lysate was loaded and electrophoresed onto NuPAGE 4-12% Bis-Tris Gel (Novex, Life Technologies) and transferred to polyvinylidene difluoride membranes (Immobilon, Millipore). Primary antibodies used in this study, all at 1:2000, were phospho-S6 (p-S6; S235/236; Cell Signaling Technology, #4858), S6 (Cell Signaling Technology, #2217), Erk1/2 (P44/42 MAPK, Cell Signaling Technology, #4695P), p-Erk1/2 (Phospho-P44/42 MAPK, Cell Signaling Technology, #9101), p-S6K (Cell Signaling Technology, #9234S), S6K (Cell Signaling Technology, #2708P), mTOR (Cell Signaling Technology, #2983S), p-mTOR (Cell Signaling Technology, #5536S), p-TrkB (Y816; Millipore, #ABN1381), TrkB (Cell Signaling Technology, #4603), p-Cdc2 (Y15; Cell Signaling Technology, #9111S), Cdc2 (Cell Signaling Technology, #77055), Map2 (Millipore, #AB5622), Tau46 (Cell Signaling Technology, #4019S), and β-actin (Cell Signaling Technology, #4970). Peroxidase-conjugated anti-rabbit or mouse IgG secondary antibody (1:5000, Jackson ImmunoResearch Laboratories, Inc.) was used. Proteins were then visualized after addition of WesternBright Quantum kit (Advansta), imaged using chemiluminescence, and quantified using ImageJ software. Quantification was performed by using the blot tool to measure relative abundance. Phosphorylated proteins were normalized to their total protein abundance (i.e., p-mTOR divided by mTOR).

In vitro primary neuronal culture: Brains from P0 mice were removed, and whole cortices were dissected. Individual cortices were placed into 1 mL chilled dissecting media (1× HBSS (14175-095), 30 mM glucose, 60 μM HEPES). After aspiration of the dissecting media, the tissue was enzymatically dissociated with papain (LS003126) that was previously diluted 1:80 in pre-warmed plating media [Neurobasal A (10888-022), 5% FBS (16000044), and 1% penicillin/streptomycin (p/s)/0.25% glutamine (10378-016)] and incubated at 37° C. for 15 min. The papain was removed, and the tissue was pipetted into a single-cell suspension in plating media. The suspensions were filtered using 40 μm nylon cell strainers (Fisher Scientific, #352340), and the filtered suspension was centrifuged at 280 RCF for 5 min. The supernatant was aspirated, and the pellet was re-suspended in pre-warmed plating media. Samples of the suspensions were diluted 1:4 in trypan blue, and cells were counted with a manual hemocytometer for determination of sample concentration. The cells were then plated at 50,000 cells per well in sterile 12-well plates (#07-200-82) containing Poly-D-Lysine (PDL) coated coverslips (GG-18-pd1). On the first day-in-vitro (DIVO), the plating medium was fully exchanged with 1 mL feeding medium [Neurobasal A, 1% penicillin/streptomycin/0.25% glutamine, 2% B-27™ serum-free supplement (17504-044)]. At DIV3, the volume of feeding media was doubled by adding 1 mL of feeding medium to all wells, and every three DIV, 1 mL of media was replaced with 1 mL fresh feeding media until DIV15. Cells were washed with 1×PBS and fixed at DIV15 with 4% paraformaldehyde (PFA) in 1×PBS for 10 min. at room temperature. After undergoing three additional washes in 1×PBS to remove remaining PFA, cells were stored in PBS at 4° C. until further use.

For (1-3)IGF-1 treatment, neurons were dosed with either Milli-Q water as vehicle or 100 ng/mL of (1-3)IGF-1 (Art. No. 40261041000; CAS No. 32302-76-4) dissolved in Milli-Q water as drug, both sterilized with the Steriflip® Vacuum Driven Filtration System. Dosage was based on previous studies (13). Cells were dosed by removing 1 mL feeding media and adding 1 mL of vehicle or drug diluted in feeding media for three consecutive days with 24-hour incubation periods in between each dose and a 12-hour incubation period between the final dose and fixation of the cells at DIV15.

For immunocytochemistry, cells were incubated with primary antibodies overnight at 4° C. in a humidity chamber. Primary antibodies used include Tuj 1 (1:750, Abcam #ab78078) and Ctip2 (1:1000, Abcam #ab18465). The secondary antibodies were diluted in PBS blocking solution and incubated with cells for 1 hour at room temperature. After washing off remaining antibody with 1×PBS, the coverslips were removed from the wells and allowed to dry for 5 min. before being mounted onto slides (48311-703) with Vectashield mounting medium containing DAPI (Life Technologies #P36935).

All coverslip images were taken at 40× magnification on an Olympus VS-120 epifluorescence microscope and analyzed with ImageJ (NIH). The Simple Neurite Tracer plugin was used to draw neurite traces for individual neurons. The Sholl analysis plugin was used to run Sholl analysis on the reconstructed neurite traces after thresholding the images to a binary format.

Statistical Analysis: Power analysis was used to determine sample size using the control mean determined from preliminary data with an alpha of 0.05 and a power of 80%. Planned comparisons between WT and mutant mice were performed for all assays using independent-sample t tests. One-way analyses of variance (ANOVAs) were used to assess genotype effect in experiments containing control, cHet, and cKO or control, cHet, and dHet mice with Tukey's or Sidak's post hoc tests used where appropriate. All statistics were performed using GraphPad Prism, with significance set at P<0.05. Throughout the application, values represent means; error bars indicate SEMs; N values refer to biological replicates. All measurements and testing were performed blind to the genotype and/or experimental manipulations.

### Example 7. Chronic (1-3)IGF-1 Treatment Rescues Cortical Undergrowth in Dyrk La cHets

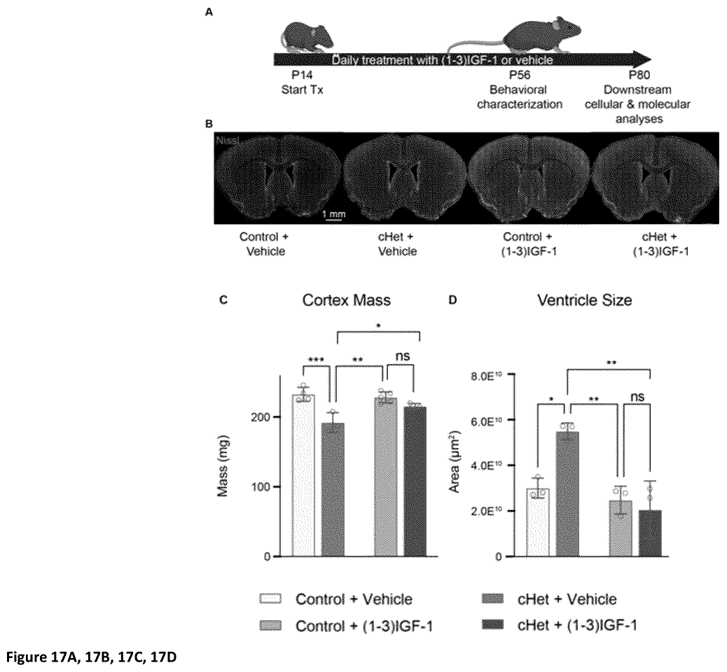

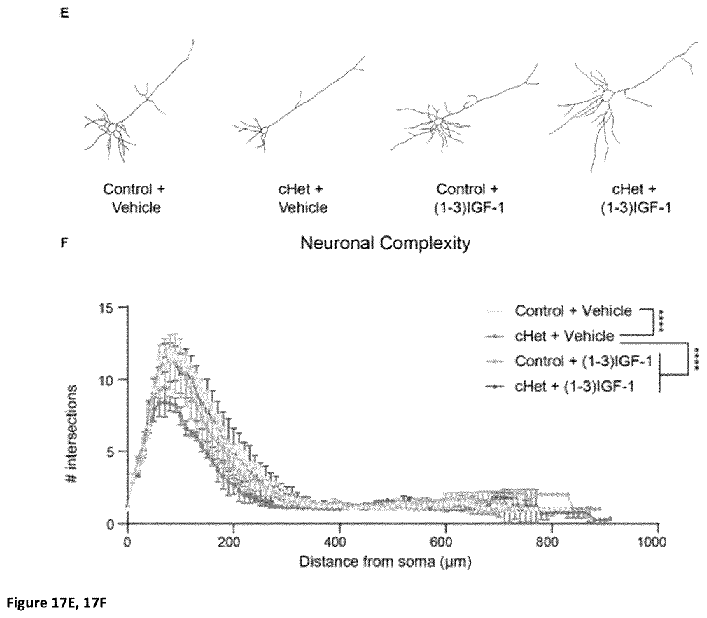

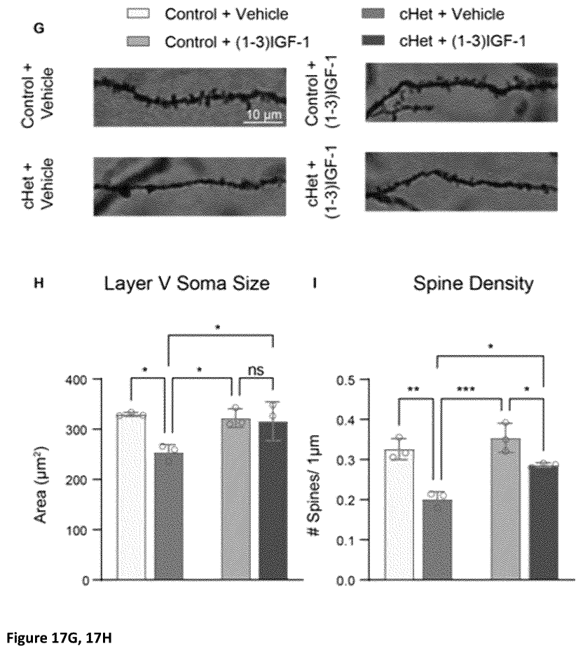

We previously showed that cortical heterozygous mutations in Dyrk1a driven by the Emx1-Cre promoter (Emx1-cre⁺; Dyrk1a^{loxP/+}, or cHet) are sufficient to cause microcephaly and ASD-relevant behaviors in mice. To test whether administration of the active peptide (1-3)IGF-1 restores cortical growth in cHets, we utilized the following paradigm: controls and cHets were treated daily with either (1-3)IGF-1 or vehicle beginning at P14. A behavioral battery began at P56 and daily injections continued until the mice were sacrificed for downstream analysis (FIG. 17A). Vehicle-treated cHets exhibited microcephaly, similar to what we have previously shown in untreated animals. Daily (1-3)IGF-1 treatment rescued cortical growth in cHets while having no effect on controls (FIG. 17C). Adult cHets exhibit enlarged ventricles, aligning with a common finding amongst patients with DYRK1A mutations. This observed enlargement in vehicle-treated cHets was normalized upon (1-3)IGF-1 treatment, with the ventricle area comparable to both vehicle- and (1-3)IGF-1-treated controls (FIGS. 17B, D). We next investigated the neuronal complexity of layer V neurons in the medial prefrontal cortex (mPFC), as we have previously shown this population of projection neurons to exhibit decreased neuronal complexity. Neuronal reconstruction and Sholl analysis revealed (1-3)IGF-1 treatment restored the neuronal complexity of layer V neurons (FIGS. 17E, F). As the impact of loss-of-function Dyrk1a mutations on spine density has not been investigated in vivo, we measured spine density on primary neurites of layer V pyramidal neurons in the mPFC. Vehicle-treated cHets exhibit decreased spine density, and this is significantly increased by (1-3)IGF-1 treatment (FIGS. 17G, I). Lastly, we found that (1-3)IGF-1 treatment restored the significantly smaller neuronal soma size of layer V pyramidal neurons, expanding what we had previously observed with neonatal (1-3)IGF-1 treatment (FIG. 17H). Together, these findings indicate that chronic (1-3)IGF-1 treatment beginning at P14 rescues microcephaly and neuronal undergrowth in Dyrk1a cHets.

FIG. 17 legend: Chronic (1-3)IGF-1 increases cortex mass and morphological abnormalities in Dyrk1a cHets (A) Treatment paradigm used for chronic administration of (13)IGF-1. (B) Representative coronal sections stained with DAPI (blue) and Nissl (green). (C) Cortex mass is decreased in vehicle-treated cHets and restored with (1-3)IGF-1 treatment. Analyzed by two-way ANOVA F_{(1,11)}=7.610, P=0.0186. (D) Ventricle area is increased in vehicle-treated cHets and rescued by (1-3)IGF-1 treatment. Analyzed by two-way ANOVA F_{(1,8)}=11.13, P=0.0103. (E) Representative images of reconstructed layer V pyramidal neurons in the mPFC. (F) Sholl analysis of layer V pyramidal neurons shows a decrease in neuronal complexity of vehicle-treated cHet neurons, which is rescued by (1-3)IGF-1 treatment. Analyzed by two-way ANOVA of the area under the Sholl curve F_{(1,8})=49.9, P=0.0001. (G) Representative 63× images of spines on layer V pyramidal neurons (H) Soma size of layer V pyramidal neurons is decreased in vehicle-treated cHets and rescued by (1-3)IGF-1 treatment. . Analyzed by two-way ANOVA F_{(1,8})=6.884, P=0.0305. (I) Spine density per 1 um is decreased in vehicle-treated cHets and partially restored by (1-3)IGF-1 treatment. Analysis revealed a trend towards a significant interaction of treatment X genotype (two-way ANOVA F_{(1,8})=4.051, P=0.079) and planned comparisons are shown on the figure. N=3-5. Significance on figure shows results after Tukey's post-hoc analysis. #P<0.1, *P<0.05, **P<0.01, ***P<0.001.

### Example 8. Chronic (1-3)IGF-1 Treatment Rescues Sociability Deficits in Dyrk La cHets

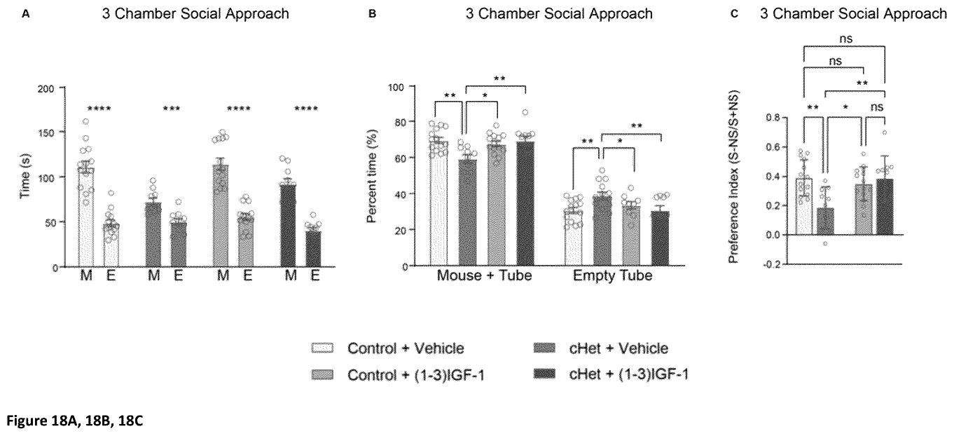

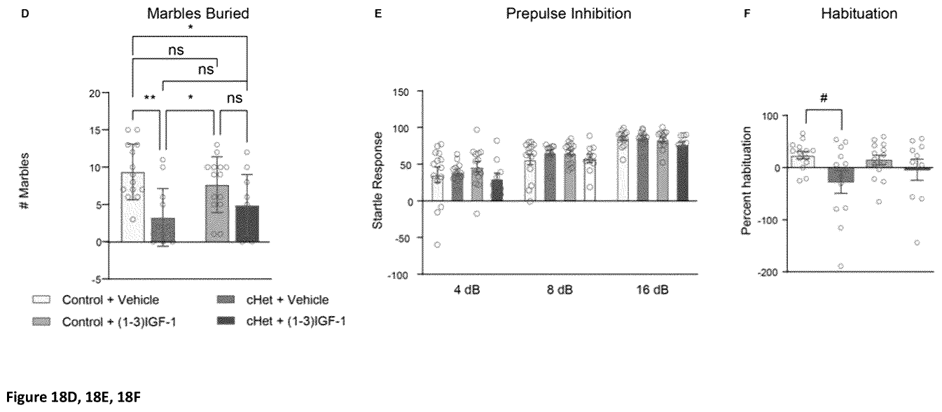

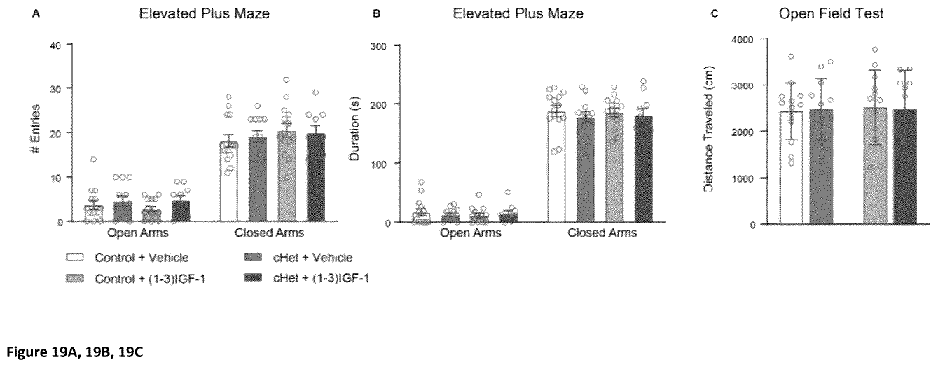

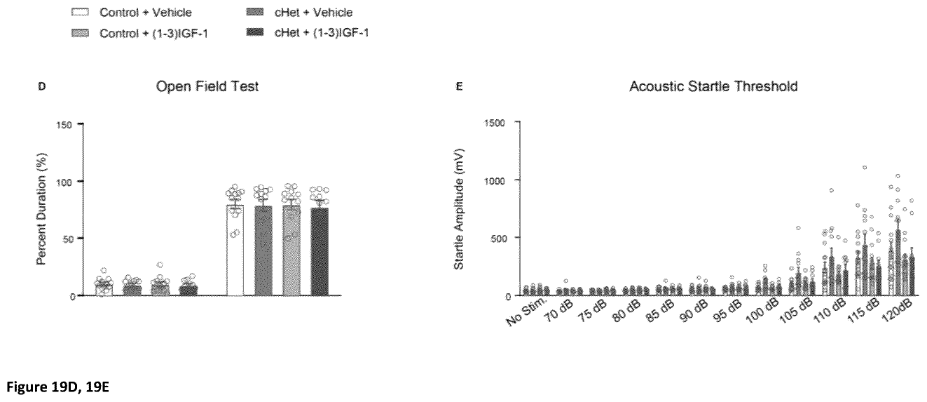

To determine whether chronic (1-3)IGF-1 administration ameliorates ASD-relevant behaviors, we conducted a behavioral battery to assay sociability and repetitive behaviors as well as locomotion, anxiety-like behavior, acoustic startle, and sensorimotor gating. The 3-chamber social approach assay revealed that all groups, including vehicle-treated cHets, exhibit a significant preference for interaction with a stimulus mouse compared to an inanimate object, contradictory to our previous findings of untreated cHets lacking this social preference (FIG. 18A). However, when compared across genotypes and drug treatments, vehicle-treated cHets spend significantly less time interacting with the stimulus mouse, as shown by both the percentage of time spent investigating and the preference index (FIGS. 18B, C). (1-3IGF-1 treatment restored this social deficit in cHets and did not impact controls. Untreated cHets exhibit decreased marble burying and qualitatively fail to interact with the marbles during the assay. While we observed no effect of (1-3)IGF-1 treatment on the number of marbles buried by cHets, qualitatively interaction with the marbles increased in cHets, suggesting a modest modification of this phenotype (FIG. 18D). Assaying of sensorimotor gating using the Prepulse Inhibition (PPI) assay revealed no difference in startle response within any genotype or treatment group (FIG. 18E). While not significant by two-way ANOVA, planned comparisons showed vehicle-treated cHets exhibit a trend towards decreased habituation to the prepulse, and this trend is not present in the (1-3)IGF-1 treated cHet group (FIG. 18F). To control for behavior that may be altered by chronic injections, we surveyed and found no effects of genotype or drug treatment in locomotion or thigmotaxis measured by the Open Field Test (OFT), anxiety-like behavior measured by the Elevated Plus Maze test (EPM), or acoustic startle behavior as measured by the Acoustic Startle Threshold (AST) (FIGS. 19A-19E). Taken together, (1-3)IGF-1 treatment ameliorates morphological, neuronal, and behavioral deficits caused by cortical Dyrk1a mutations.

FIG. 18 legend: Sociability deficits and marble burying are partially ameliorated by chronic (1-3)IGF-1 treatment (A) 3 chamber social approach assay shows all genotypes X drug treatments have a significant preference for social interaction. Analyzed by paired t-tests. (B) Social interaction time hand-scored and compared between genotypes and drug treatments shows vehicle-treated cHets interact significantly less with a social stimulus and this is increased by (1-3)IGF-1 treatment. Analyzed by two-way ANOVA: Social/non-social interaction F_{(1, 44)}=9.561, P=0.0034. (C) Preference index, measured by the formula indicated, shows vehicle-treated cHets have a significantly decreased PI compared to all other groups and this is restored by (1-3)IGF-1 treatment. Analyzed by two-way ANOVA F_{(1, 44})=9.561, P=0.0034. (D) There is no significant interaction of treatment X genotype for the number of marbles buried (F_{(1,46)}=2.349, P=0.1322). However, planned comparisons show a significant decrease in vehicle-treated cHets and subtle modification of the phenotype by (1-3)IGF-1. (E) There is no difference in the (E) startle response or (F) habituation of any treatment X genotype within the prepulse inhibition assay. Startle response analyzed by repeated measures three-way ANOVA showed no interaction of genotype X decibel or dB X genotype X treatment but did show a significant effect of decibel: F_{(6, 92})=0.5319, P=0.7827. Habituation analyzed by two-way ANOVA show no significant interaction between genotype X treatment but do show a significant effect of genotype: F_{(1,46)}=1.271, P=0.2655. Planned comparisons show a trend towards decreased habituation in vehicle-treated cHets. N=10-14. Significance on figure shows results after Tukey's post-hoc analysis. #P<0.1, *P<0.05, **P<0.01, ***P<0.001.

### Example 9. (1-3)IGF-1 Treatment Increases Reduced Protein Synthesis in the mPFC of cHets

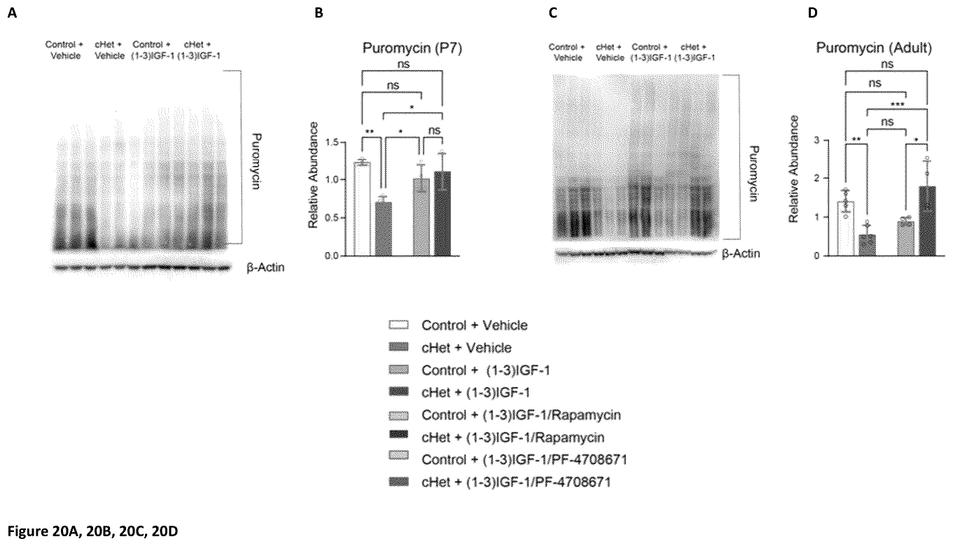

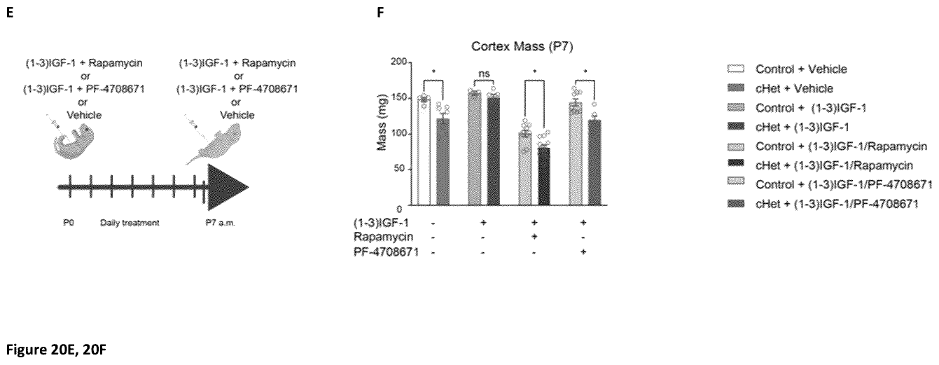

Protein synthesis is a critical output of mTOR signaling, particularly downstream of P70 Ribosomal S6 Kinase (S6K1). As cHets exhibit altered mTOR signaling and specifically decreased phosphorylation of S6K1, we next tested whether protein synthesis is altered in neonatal cHets using the surface sensing of translation (SUnSET) puromycin incorporation assay. Protein lysate from the mPFC of neonatal vehicle-treated cHets showed decreased puromycin incorporation normalized to a loading control. Daily treatment from P0 to P7 with (1-3)IGF-1 increased puromycin abundance without impacting controls (FIGS. 20A, B). We observed a similar decrease in puromycin incorporation in adult vehicle-treated cHets, and this was significantly increased by (1-3)IGF-1 treatment. Interestingly, we observed a significant decrease in puromycin incorporation in (1-3)IGF-1 treated controls relative to their vehicle-treated counterpart (FIGS. 20C, D).

While IGF-1 signaling downstream of the IGF-1 receptor (IGF-1R) is well understood, it is not yet known how administration of the IGF-1R ligand impacts growth and protein synthesis. Several effectors downstream of IGF-1/IGF-1R play critical roles in regulating growth and protein synthesis including mTOR and S6K1. To determine whether these molecules are critical for the therapeutic effects of (1-3)IGF-1, we co-administered (1-3)IGF-1 and either mTOR inhibitor Rapamycin or S6K1 inhibitor PF-4708671 in the same neonatal paradigm previously used. Co-administration of (1-3)IGF-1 and Rapamycin failed to rescue cortex mass in cHets, with cHets exhibit significantly decreased cortex mass compared to treated controls. It is important to note that (1-3)IGF-1+Rapamycin administration significantly impacted the body mass of the pups, aligning with previous findings from rapamycin studies in neonatal mice (33A). Co-administration of (1-3)IGF-1 and PF-4708671 failed to rescue cortex mass in cHets as well (FIG. 20). These data indicate that both mTOR and S6K1 are critical effectors of IGF-1 and provide novel insight for the therapeutic benefits of IGF-1 treatment (FIGS. 20E-G).

FIG. 20 Legend: (1-3)IGF-1 administration restores decreased protein synthesis in neonatal and adult cHets. (A) Representative western blot probing for puromycin and loading control and (B) Quantification. Analyzed by two-way ANOVA F_{(1,10)}=16.47, P=0.0019. (C) Representative western blot probing for puromycin and loading control and (D) Quantification. Analyzed by two-way ANOVA F_{(1, 15})=28.92, P<0.0001. (E) Neonatal treatment paradigm. (F) Co-administration of (1-3)IGF-1 with either Rapamycin or PF-4708671 fails to rescue cortex mass in P7 cHets. Analyzed by two-way ANOVA F_{(3, 50})=0.9727, P<0.4131 shows no significant interaction of treatment X genotype but does show significant effects of both treatment and genotype. Planned comparisons shown. N=3-15. Significance on figure shows results after Tukey's post-hoc analysis. #P<0.1, *P<0.05, **P<0.01, ***P<0.001.

### Example 10. GSK3β is a Hyperactive Kinase in cHets

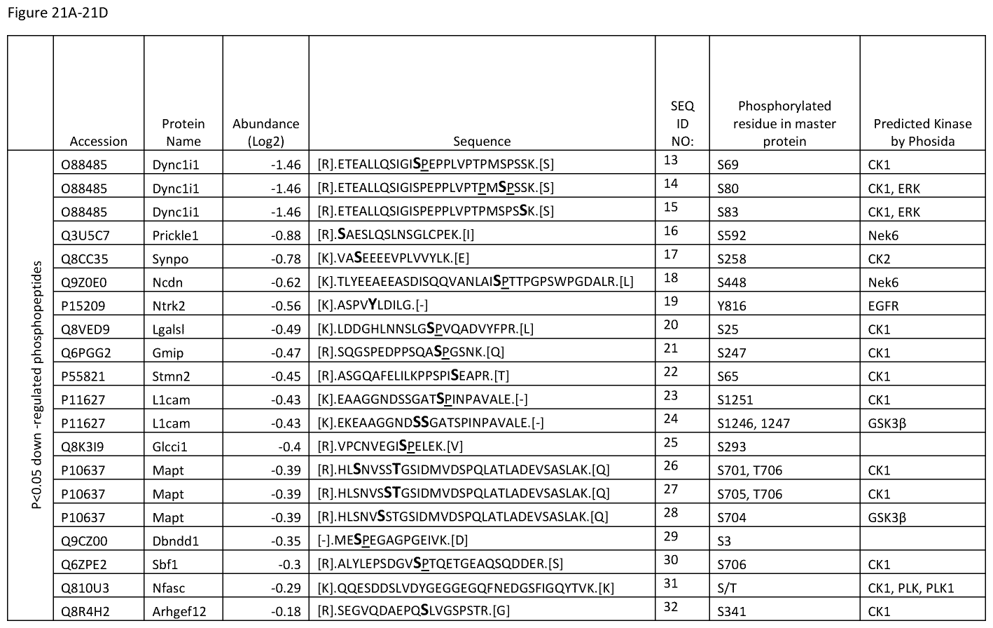

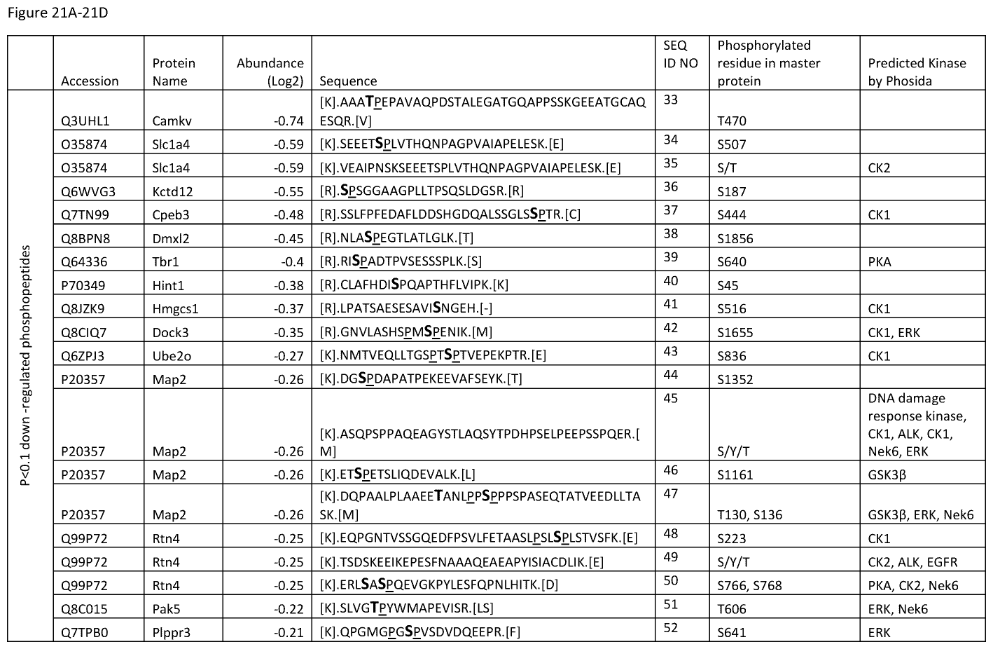

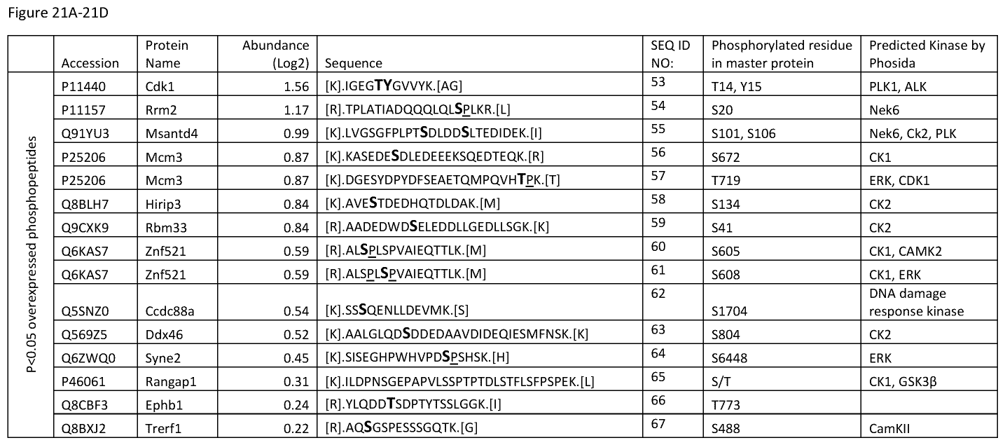

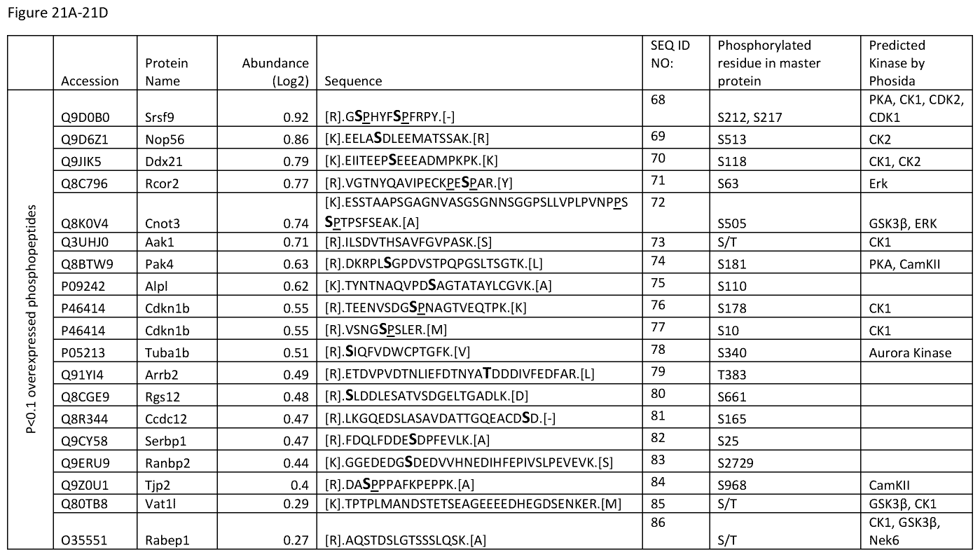

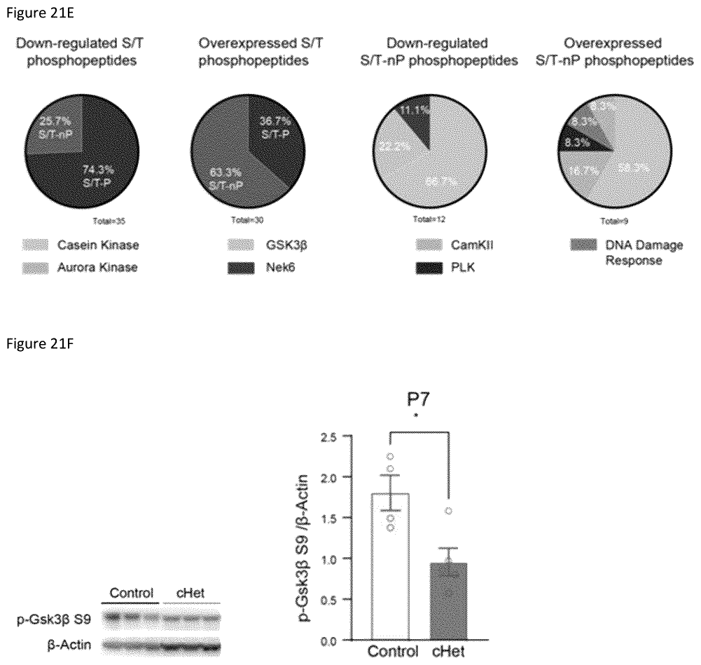

Given the therapeutic effectiveness of (1-3)IGF-1 in ameliorating undergrowth and sociability deficits in cHets, we next aimed to target a second effector within the canonical growth signaling cascade downstream of IGF-1R that would elicit similar beneficial effects. Previously conducted phosophoproteomics revealed several altered targets in the Dyrk1a mutant cortex. Given Dyrk1a is a Serine/Threonine kinase, we surveyed the altered phosphopeptides for the Dyrk1a consensus sequence RPx(S/T)P to determine whether any phosphopeptides were likely direct targets of Dyrk1a kinase activity (34A). Interestingly, none of the altered phosphopeptides contained the exact consensus sequence of Dyrk1a. Overlap within consensus sequences of several kinases is highly abundant and it has been previously shown that consensus sequences are not a perfect determinant of direct kinase activity (35A, 36A). Moreover, the Arginine (R) in the −3 position and Proline (P) in the +1 position were shown to be the minimal amino acids required for phosphorylation by Dyrk1a (37A). We separated the altered phosphopeptides based on whether they contain a Proline in the +1 position (denoted S/T-P) or non-Proline in the +1 position (denoted S/T-nP). As they contain the critical Proline in the +1 position, S/T-P phosphopeptides may be targets of Dyrk1a kinase activity and represent the majority of altered targets in the down-regulated group (74.3%) versus being the minority in the overexpressed group (36.7%) (FIGS. 21A-E). We next sought to determine which kinases are responsible for the altered phosphorylation of each peptide using the kinase predictor algorithm PHOSIDA (38A, 39A). Within the S/T-nP group, Casein Kinases are likely responsible for the majority of dysregulated phosphopeptides in both the down-regulated and overexpressed groups (FIG. 21E). In the overexpressed ST/n-P group, other identified kinases include Calmodulin Kinase II (CamKII), Polo-like Kinase (PLK), DNA Damage Response (DDR) kinase, and Aurora Kinase. In the down-regulated S/T-nP group, only GSK3β and NIMA Related Kinase 6 (Nek6) were identified. Aside from Casein Kinase, which is the most highly represented predicted kinase in both downregulated and overexpressed phosphopeptide groups, the most frequently identified predicted kinase specific to one group is GSK3β (FIG. 21E). We directly tested GSK3β activity by probing cortical lysate from P7 cHets. Indeed, inhibitory phosphorylation of GSK3β on Serine 9 (S9) is decreased in cHets, indicating hyperactivity of GSK3β (FIG. 21F).

FIG. 21 Legend: GSK3β is hyperactive in cHets. (A-D) Table of significant and marginally significant down-regulated (A-B) and overexpressed (C-D) phosphopeptides in the Dyrk1a cKO cortex at birth. The phosphorylated residue is denoted in bold in larger font within the sequence. If no residue is highlighted and the P-residue is listed as “S/T”, this denotes that the analysis software was unable to confidently determine which Serine or Threonine was phosphorylated within the specific sequence. A residue that aligns with Dyrk1a consensus sequence (typically either R in −3 position, P in −2 position, or P in +1 position) is denoted by underlining within the sequence. Residues in brackets: the amino acid before/after the identified sequence. A dash in brackets before a sequence=beginning of the sequence, no amino acid residue beforehand. A dash in brackets after a sequence=end of the sequence, no amino acid residue afterwards. Predicted kinase was established using PHOSida software. (E) The majority of down-regulated phosphopeptides contain a Proline (P) in the +1 residue, aligning with the Dyrk1a consensus sequence. Of the sequences that do not contain a P in the +1 residue, termed “ST-nP”, the majority are predicted to be targeted by Casein Kinase. GSK3β is predicted to target ˜22% of phosphopeptides specifically in the down-regulated ST-nP group. (F) Western blot of the P7 cortex shows inhibitory phosphorylation of GSK3β on Serine 9 (S9) is decreased in cHets, concurrent with the predicted hyperactivity of GSK3β. Analyzed by unpaired t-test. N=3-5. #P<0.1, *P<0.05, **P<0.01, ***P<0.001.

### Example 11. Genetic Suppression of GSK30 Rescues Microcephaly and ASD-Relevant Behaviors

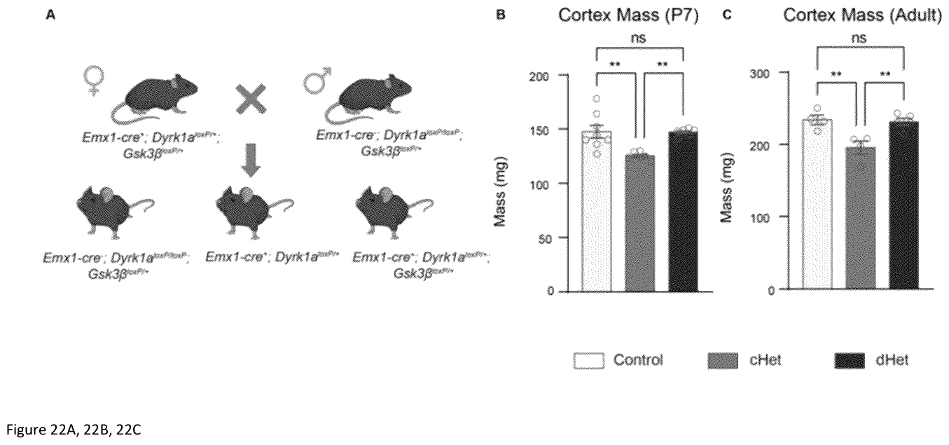

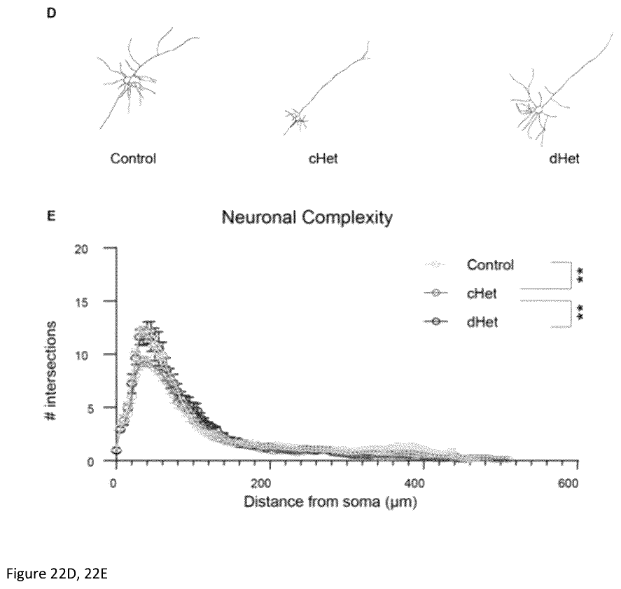

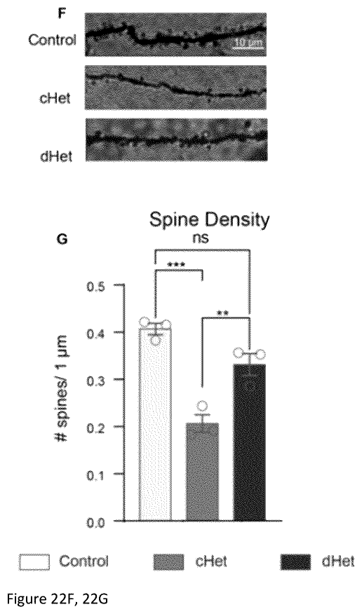

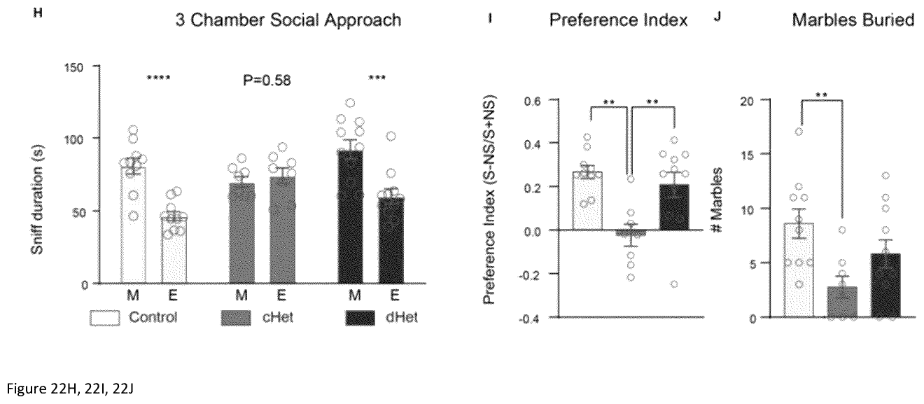

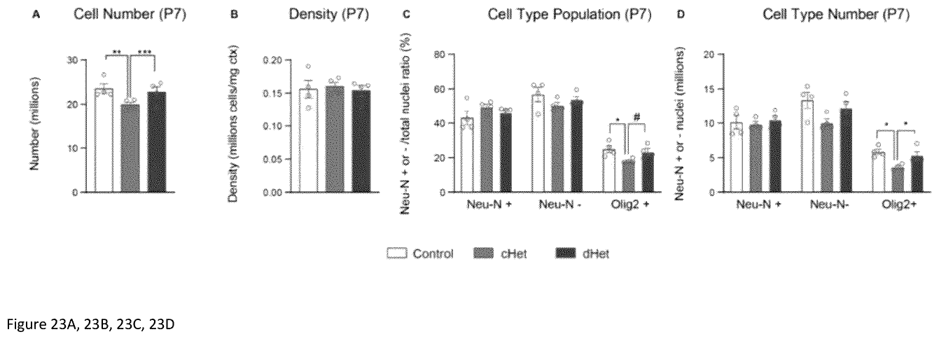

To determine whether hyperactivity of GSK3β contributes to the pathobiology of Dyrk1a mutations, we genetically suppressed Gsk3β in cHets (Emx1-cre⁺; Dyrk1a^{loxP/+}; Gsk3β^{loxP/+}, or dHet)(FIG. 22A). Genetic suppression of Gsk3β resulted in normalized cortex mass in both neonatal and adult cHets (FIGS. 22B, C). As we have previously shown that Dyrk1a mutations cause cortical undergrowth via reduced cortical cell number and because of the critical role of GSK3β in regulating proliferation, we tested whether genetic suppression of Gsk3β rescues the observed decreased cellular number. Using isotropic fractionator to dissociate nuclei and subsequent staining of the nuclei with neuronal marker Neu-N and oligodendrocyte marker Olig2, we found that dHets exhibit normalized cellular number in both P7 and adult cHets. Because the delta in cortical mass aligns with the delta in cellular number, neither genotype exhibits altered cellular density. While there was no change in the population of Neu-N+ or Neu-N− cells, flow cytometry of dissociated nuclei revealed a decrease in the percentage and absolute number of Olig2+ cells in P7 cHets. This proportional and absolute decrease in Olig2+ cells is normal in dHets. In adult cHets, the proportion of Neu-N+, Neu-N− and Olig2+ cells are unchanged. These proportions calculated against the cortical cellular number revealed that the absolute number of both Neu-N+ and Neu-N populations are decreased in adult cHets, and both cell types are rescued in number in dHets. Together, these data show that genetic suppression of Gsk3β rescues cortical undergrowth via increased cellular number of both neuronal and non-neuronal populations (FIGS. 23A-23H).

To determine whether the observed decreased neuronal complexity is restored in dHets, we measured layer V pyramidal neurons after neuronal reconstruction and Sholl analysis. dHets also exhibit neuronal complexity of layer V pyramidal neurons in the mPFC comparable to controls (FIGS. 22D, E). Similar to the therapeutic effect of (1-3)IGF-1, genetic suppression of Gsk3β also fully restored spine density (FIGS. 22F, G). We next tested sociability in dHets in the 3-chamber social approach assay. To date, all behavioral analysis conducted on Dyrk1a cHets has been done in male mice. Due to the sexual dimorphism that is highly prevalent not only in patients with ASD, but in mouse models as well, we conducted the following behavioral assays in females. Surprisingly, cHets do not exhibit sexual dimorphism in their observed sociability deficit, as female cHets also lack a significant preference for social interaction and spend significantly less time with a social stimulus compared to control mice (FIGS. 22H, I). Genetic suppression of Gsk3β rescues this deficit, with dHets exhibiting a significant social preference. The calculated preference index indicates that female cHets spend significantly less time interacting with a social stimulus compared to controls, and this is fully ameliorated in dHets. In the marble burying assay, female cHets recapitulated our finding that male cHets bury significantly fewer marbles than controls. dHets did not bury a significantly different number of marbles compared to controls or cHets, suggesting a partial modification of this phenotype. Similar to the effects of (1-3)IGF-1, dHets qualitatively interacted with the marbles more so than cHets (FIG. 22J). FIGS. 24A and 24B indicate that Anxiety-like behavior and locomotion are unaltered in cHets and dHets.

FIG. 22 Legend: Genetic suppression of GSK3β rescues microcephaly, autism-relevant behaviors, and neuronal complexity/spine density in cHets. (A) breeding scheme used to generate double mutants. (B) Cortex mass is decreased in P7 cHets and comparable to controls in dHets. Analyzed by one-way ANOVA F_{(2,17})=8.58, P=0.0027. (C) Cortex mass is decreased in adult cHets and comparable to controls in dHets. Analyzed by one-way ANOVA F_{(2,10})=9.37, P=0.0051. (D) Representative images of reconstructed layer V pyramidal neurons in the mPFC. (E) Sholl analysis of layer V pyramidal neurons shows a decrease in neuronal complexity of cHet pyramidal neurons, which is rescued by genetic suppression of Gsk3, 8. Analyzed by one-way ANOVA of the area under the Sholl curve F_{(2, 6})=32.12, P=0.0006. (F) Representative 63× images of spines on layer V pyramidal neurons (G) Spine density per 1 um is decreased cHets and is restored by genetic suppression of Gsk3, 8. Analyzed by one-way ANOVA F_{(2,6})=29.5, P=0.0008. (H) In the 3 chamber social approach assay, cHets do not exhibit a significant preference for social interaction, and this is restored in cHets. Analyzed by paired t-tests. (I) Preference index, measured by the formula indicated, is decreased in cHets compared to controls and dHets. Analyzed by one-way ANOVA F_{(2, 26})=9.163, P=0.001. (J) cHets bury significantly fewer marbles compared to controls, and this is partially restored in dHets as they do not bury significantly different number of marbles compared to controls or cHets. Analyzed by one-way ANOVA F_{(2, 26})=4.883, P=0.0158. N=3-8 for morphology analysis, N=8-12 for behavior. Significance on figure shows results after Tukey's post-hoc analysis. #P<0.1, *P<0.05, ** P<0.01, ***P<0.001.

### Example 12. Pharmacological Inhibition of GSK3β Rescues Microcephaly in Neonatal cHets

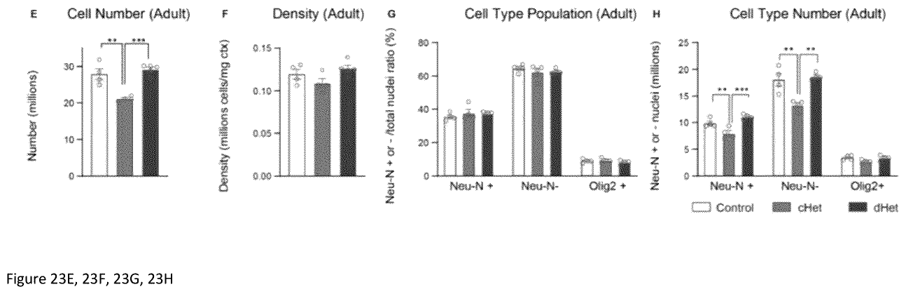

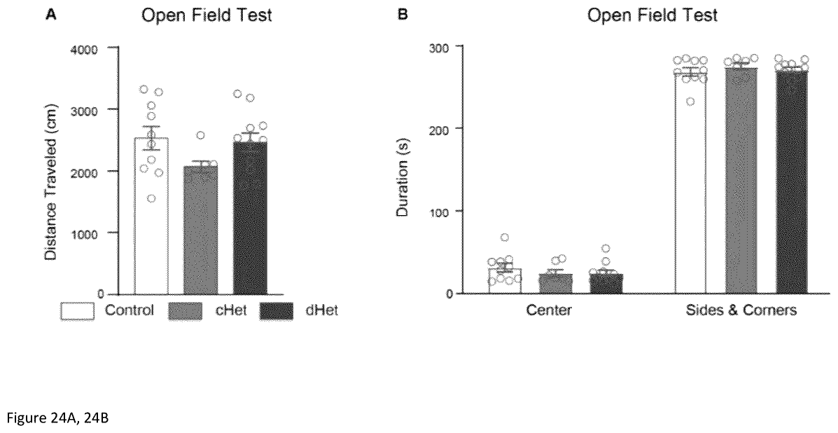

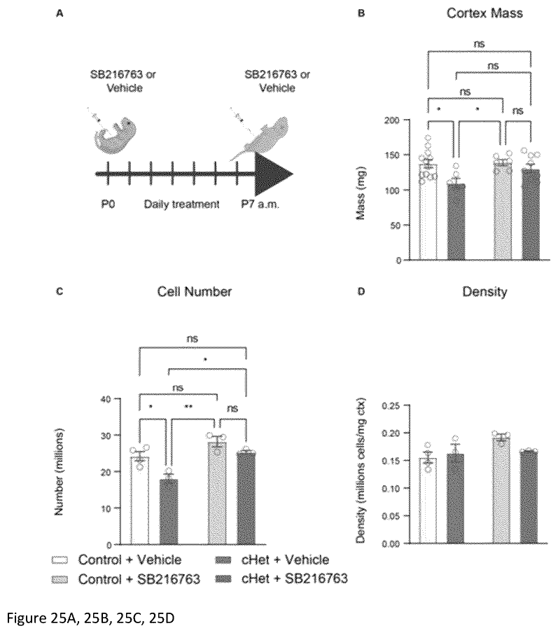

With the promising therapeutic effect of genetic suppression of Gsk3β in rescuing microcephaly caused by Dyrk1a mutations, we next sought to determine whether a pharmacological approach would elicit the same advantageous results. We utilized the previously mentioned neonatal paradigm to inject cHets daily with GSK3 inhibitor SB216763 or vehicle from P0 to P7 (FIG. 25A). Daily administration of SB216763 resulted in normalized cortex mass in cHets compared to vehicle-treated controls, with no effect of SB216763 treatment on controls (FIG. 25B). Moreover, SB216763 administration restored the decreased cellular number observed in vehicle-treated cHets with no effect on cellular density, providing a potential cellular mechanism of rescue (FIGS. 25C, D).

FIG. 25 Legend: Pharmacological inhibition of GSK3β rescues microcephaly in neonatal cHets. (A) Neonatal treatment paradigm used. (B) Cortex mass is decreased in vehicle-treated cHets and this is restored by treatment with SB216763. Controls are unaltered by treatment. Analysis by two-way ANOVA, F_{(1,29})=2.165, P=0.1520 shows no significant interaction between genotype and treatment, however there is a significant effect of treatment and a trend towards an effect of genotype. Planned comparisons are shown on the figure. (C) Cortical cell number is decreased in vehicle-treated cHets and this is restored by SB216763 treatment with no effect on SB216763-treated controls. Analysis by two-way ANOVA, F_{(1,9)}=1.860, P=0.2057 shows no significant interaction between genotype and treatment, however there is a significant effect of both treatment and genotype. Planned comparisons are shown on the figure. (D) Cellular density is unaltered by any genotype or treatment. Analyzed by two-way ANOVA, F_{(1,9)}=2.665, P=0.1370. Significance on figure shows results after Tukey's post-hoc analysis. N=3-12. #P<0.1, *P<0.05, **P<0.01, ***P<0.001.

### Example 13. Genetic Suppression of Gsk3b Rescues Decreased Protein Synthesis in Neonatal and Adult cHets

GSK3β signaling is integrated into canonical growth factor signaling via TSC2 and is inhibited by both ERK/Akt and S6K1(40A, 41A). Furthermore, GSK3β regulates protein synthesis via phosphorylation and inactivation of eukaryotic initiation factor eIF2B (42A). To determine whether enhanced protein synthesis is a common mechanism of rescue between both (1-3)IGF-1 treatment and genetic suppression of Gsk3J3, we measured puromycin incorporation via the SUnSET assay in dHets. cHets exhibit decreased protein synthesis, and this is rescued and partially rescued in dHets at P7 and adulthood, respectively (FIGS. 26A-D).

FIG. 26 Legend: Genetic suppression of Gsk3b rescues decreased protein synthesis in neonatal and adult cHets. (A) Representative western blot probing for puromycin and loading control and (B) Quantification. Analyzed by one-way ANOVA F_{(2,8)}=12.46, P=0.0035. (C) Representative western blot probing for puromycin and loading control and (D) Quantification. Analyzed by one-way ANOVA F_{(2,8)}=5.786 P=0.0279 . . . N=3-5. Significance on figure shows results after Tukey's post-hoc analysis. #P<0.1, *P<0.05, **P<0.01, ***P<0.001.

### Example 14. Materials and Methods for Examples 7-13 Mice

All mice used in the study were obtained from the Jackson Laboratory (Bar Harbor, ME) and have been previously described, including C57BL/6-Dyrk1a^{tm1Jdc}/J (Dyrk1a^{loxP/loxP}, stock #027801), Emx1^{tm1 (cre)Krj} (Emx1-cre^{+/-}, stock #005628), and B6.129(Cg)-Gsk3b^{tm2Jrw}/J (Gsk3b^{loxP/loxP}, stock #029592). Wild-type and Emx1-cre⁻; Dyrk1a^{loxP/+} mice were used as controls. To avoid germline recombination in males, Emx1-cre⁻; Dyrk1a^{loxP/loxP} males were bred with Emx1-cre⁺; Dyrk1a^{loxP/+} females. Genomic DNA isolated from ear samples was used for polymerase chain reaction to confirm genotypes. All animal experiments were conducted in accordance with National Institutes of Health and Association for Assessment and Accreditation of Laboratory Animal Care guidelines and were approved by The Scripps Research Institute's Institutional Animal Care and Use Committee. Mixed sexes were used for all cellular and molecular assays.

Drug Treatments

(1-3)IGF-1 was acquired from VWR/Bachem (catalog #H-2468.1000) and dissolved in saline with 0.01% BSA fresh every day. Intraperitoneal injections of 10 mg kg′ (1-3)IGF-1 or vehicle were delivered daily at 8 a.m. starting on P14 and ending on P85. This dosage was chosen based on multiple studies of (1-3) IGF-1 treatment in ASD mouse models that showed brain penetrance and increased signaling downstream of IGF-1 within 3 hours post-injection. The S6K1 inhibitor PF-4708671 was acquired from Medkoo Biosciences (#406432) dissolved in 10% DMSO, 10% tween 80 and 80% water. (1-3)IGF-1 (10 mg kg′) and PF-4708671 (50 mg kg⁻¹) or vehicle were injected subcutaneously into pups from P0 to P7. Rapamycin was acquired from LC laboratories (R-5000) and dissolved in 4% ethanol, 5% tween-80, and 5% PEG400 in PBS. (1-3)IGF-1 (10 mg kg′) and Rapamycin (1 mg kg′) or vehicle were injected subcutaneously into pups from P0 to P7. SB216763 was acquired from TOCRIS (#1616) and dissolved in 5% DMSO and 0.9% saline in dH20. SB216763 (0.5 mg kg′) was injected subcutaneously into pups from P0 to P7.

Behavior

3 chamber social approach: Briefly, after at least 2 weeks housing under reversed light cycle, each mouse was habituated in the three-chamber box for 5 minutes before a stimulus mouse was placed into a tube in the “social” chamber. The stimulus mice used were young adult male mice. The time spent in each chamber (“mouse+tube”, “empty tube”, “center”) was hand-scored for sniffing time using Boris.

Marble burying: Male mice were placed individually in a cage with 5 cm of ¼″ corncob bedding and 20 black marbles (14.3 mm in diameter) arranged in a 4×5 matrix and left undisturbed for 30 min. The number of marbles that were at least two-thirds buried at the end of the trial were counted.

Elevated plus maze: For elevated plus maze test, a mouse was placed in the center of the maze to start the test. Each trial lasts for 5 min. Total distance moved and durations in open and closed arms were recorded automatically.

Open field test: Male mice were placed individually in the center of the open field arena (43.8×43.8×32.8 cm) under white light with 70 dB background white noise for 10 min. Total distance moved, velocity, and time spent in the center versus corners/sides (thigmotaxis) were recorded automatically using Ethovision XT software.

Acoustic startle threshold: Each mouse was placed inside a clear acrylic tube secured to a platform with a piezoelectric accelerometer attached 30 beneath the tube inside ventilated, sound-attenuating chambers with no house or cue lights on (San 31 Diego Instruments, San Diego, CA, USA). Following a 5 min acclimation period, mice received trials of 40 ms white noise stimuli of varying intensities (from 0-50 dB above the 70 dB background white noise by 5 dB increments). Stimuli were presented in a pseudorandom order, with 8 presentations per intensity, plus 8 control trials (no stimulus), and variable 8-23 s inter-trial intervals. The maximum whole-body flinch response to each stimulus (“startle response amplitude”) was recorded using SR-Lab software (San Diego Instruments), which takes 65 consecutive 1 ms readings from the beginning of 6 stimulus onset.

Prepulse inhibition: Each mouse was placed inside a clear acrylic tube (5 cm in diameter, 10 cm long) secured to a platform with a piezoelectric accelerometer attached beneath the tube (San Diego Instruments) inside ventilated, sound-attenuating chamber. Following a 5 min acclimation period, mice receive trials of white noise stimuli in three phases: I) baseline startle, II) mixed startle and pre-pulse inhibition, and III) final startle. During the test, trials are presented at variable 8-23 s inter-trial intervals, and 70 dB background white noise is present throughout. Phases I and III consist of 6 startle trials with a white noise stimulus of 40 ms at 120 dB. During phase II, mice receive a total of 52 trials of three types, presented in a pseudorandom order: i) 12 startle trials (as in phases I and III), ii) 10 control trials (no stimulus), iii) pre-pulse inhibition trials (20 ms pre-pulse stimulus at 4, 8, or 16 dB above background, followed by 120 dB startle stimulus 100 ms after pre-pulse onset), 10 for each pre-pulse stimulus. The maximum whole-body flinch response to each startle pulse (whether preceded by a pre-pulse or not) are recorded using SR-Lab software (San Diego Instruments), which takes 65 consecutive 1 ms readings from the beginning of stimulus onset. Response to each type of stimulus was averaged across presentations within each phase. Percent pre-pulse inhibition was calculated as [(phase II startle trial−pre-pulse trial)/(phase II startle trial)×100].

Immunohistochemistry

Adult animals were perfused with 4% PFA and pups underwent rapid decapitation and dissection. After 18 hours post-fixation in 4% PFA, brains were incubated in a 20% sucrose/PBS solution at 4° C. for 2-3 days and embedded in Tissue-Tek OCT compound (Sakura). 40 μm sections were collected on Superfrost/Plus slides and stained. DAPI was used for nuclear labeling (Invitrogen, catalog #D3571). Slides were mounted with Vectashield HardSet and images were obtained with an Olympus VS120 microscope and processed using the VS-DESKTOP software (Olympus) or ImageJ (NIH). Ventricle size was analyzed by drawing an ROI around the entire ventricle in 2-3 plane-matched sections per animal.

Isotropic Fractionator

Adult animals were perfused with 4% PFA, and pups underwent rapid decapitation and dissection. Brains were fixed in 4% PFA for one week, and then the cortex was dissected out and dissociated in a 7 ml glass tissue homogenizer (Kontes Glass) in dissociation buffer (1% Triton X-100 in 40 mM sodium citrate). 1 ml of nuclei was removed and stained with 1:50 DAPI (Invitrogen, catalog #D3571) before being loaded onto a hemocytometer (Fisher Scientific, catalog #0267110). Per animal, 6 technical replicates of 10 μl nuclei were imaged and counted in the hemocytometer (0.1 μl nuclei counted per technical replicate). After calculating the total cell number per cortex, the number of cells was divided by mg of cortex to obtain density.

Flow Cytometry

After isotropic fractionator, nuclei were stained with DAPI (1:100) and primary (anti-NeuN antibody (1:500; 104225, Abcam, anti-Olig2 (1:500, AB9610, Millipore)) and secondary (Goat anti-Mouse 488 (1:2000, A21121, Life Technologies)) antibodies, then filtered through a 30 μm diameter cell strainer (#352235, Falcon) before analysis. On a Gallios flow cytometer analyzer (Beckman Coulter), DAPI fluorescence was collected with a 450/50 filter (405 nm laser) and Alexa 488 with a 550 SP filter (488 nm laser). Background fluorescence was determined using a control with only the secondary antibody. The Neu-N+/Olig2+ population was determined by gating the population of single, DAPI+ events above background using FlowJo software. Total neuronal and non-neuronal numbers were calculated by taking the percentage (as determined by flow cytometry) of the total cortical cell number from the same biological sample.

Golgi Staining

Following rapid decapitation, brains were removed and processed using the FD Rapid Golgistain™ kit (FD Neurotechnologies, Columbia MD) following the manufacturer's instructions. A Leica vibrating microtome was used to cut 100 μm coronal sections in 6% sucrose in PBS. The sections were then mounted on gelatin-coated slides and stained using the FD Rapid Golgistain™ kit. Individual neurons were imaged using a Leica DM5500B 20× objective and reconstructed using Neuromantic software. After reconstruction, Sholl analysis was performed in ImageJ using the Simple Neurite Tracer. Primary neurites were selected for spine density analysis where the number of spines was counted and divided by the length of the neurite fragment. Soma size was measured using the ROI freehand tool on imageJ (NIH).

Surface Sensing of Translation (SUnSET)

300 μm coronal prefrontal cortical slices were obtained from postnatal day 7 (P7) and adult mice. Slices were allowed to recover in artificial cerebrospinal fluid (in mM: 124 NaCl, 2.8 KCl, 2 CaCl2, 1.25 NaH2P04, 1.25 MgSO4, 26 NaHCO₃, 10 Dextrose) for 2 h at 32° C. Puromycin (5 μg ml-1) was applied to slices for 40 min at 32° C. to label newly synthesized proteins. Slices were then snap frozen on dry ice and stored at −80° C. until used. Protein (50 μg) was loaded on 4-12% gradient gels, transferred onto polyvinylidene difluoride membranes, and probed with anti-puromycin antibody (1:5,000, Millipore, MABE343). Anti-mouse HRP-tagged secondary antibody was used and chemiluminiscence was developed using standard ECL reagents. Quantification was done by measuring intensity of each lane from 14 to 191 kDa, subtracting it from the intensity of the unlabeled lane and normalized to loading control.

Western Blot

Following rapid decapitation, cortices were dissected and homogenized in cell lysis buffer (CST) containing the Protease Inhibitor Cocktail (Roche) and phosphatase inhibitors (Sigma-Aldrich) using a dounce homogenizer. Total protein concentrations were measured using the Pierce BCA Protein Assay Kit (ThermoScientific). 50 μg of lysate were loaded and electrophoresed onto NuPAGE 4-12% Bis-Tris Gel (Novex, Life Technologies) and transferred to polyvinylidene difluoride membranes (Immobilon, Millipore). Primary antibodies used in this study, all at 1:1000, were p-Gsk3α/β S21/9 (Cell Signaling Technology, #9331s), Gsk3β (Cell Signaling Technology, #9315s), GAPDH (GeneTex, #GTX100118), and β-actin (Cell Signaling Technology, #4970). Peroxidase-conjugated anti-rabbit or mouse IgG secondary antibody (1:5000, Jackson ImmunoResearch Laboratories, Inc.) was used. Proteins were then visualized after addition of WesternBright Quantum kit (Advansta), imaged using chemiluminescence, and quantified using ImageJ software. This was done using the blot tool, measuring relative abundance.

Statistical Analysis

Power analysis was used to determine sample size using the control mean determined from preliminary data with an alpha of 0.05 and a power of 80%. One-way analyses of variance (ANOVAs) were used to assess genotype effect in experiments containing control, cHet, dHet mice with Tukey's post hoc tests. Two-way ANOVAs were used to assess genotype X treatment effects with Tukey's post hoc tests. All statistics were performed using GraphPad Prism, with significance set at P<0.05. Throughout the application, values represent means; error bars indicate SEMs; N values refer to biological replicates. All measurements and testing were performed blind to the genotype and/or experimental manipulations.

### Example 15: RNA Function Inhibitors Design and Selection

RNA function inhibitors, ASOs, siRNAs, and shRNAs, targeting the GSK3β gene or the PTen gene are designed an synthesized having complementarity to the GSK3β gene or the PTen gene. The RNA function inhibitors are designed using methods available in the art. Preference is given for sequences that are not complementary to other sequences in the human transcriptome.

Individual nucleotides in the ASOs and siRNA are modified as is typical in the art. For instance, the ASO can be synthesized as gapmers. The siRNAs can be synthesized such that every nucleotide in the sense strand and the antisense strand in modified.

Activity of the RNA function inhibitors are first analyzed in culture cells or primary cells. The RNA function inhibitors are transfected to cells. In some experiments, the cells express the target gene linked to a luciferase gene such that knockdown of target gene expression can be measure by analysis of luciferase activity in the cells. Transfected cells are incubated for a period of time, e.g., 24-24 hours, and assayed for target gene activity, mRNA abundance (RT-PCR), or luciferase activity. Single dose, dose response, and multidose samples can be analyzed and compared with control (no RNA function inhibitor or scramble sequence RNA function inhibitor) samples. Data are expressed as percent of message or activity remaining relative to untreated cells.

RNA function inhibitors exhibiting high levels of target gene knockdown in vitro are administered to animal models, such a mice, rats, dogs, and/or non-human primates. Knockdown of target gene expression is then measure in the animal models. Preference is given to RNA function inhibitors whose that inhibit expression of the target gene by more than 50%.

### REFERENCES

1. Stessman H A, Xiong B, Coe B P, Wang T, Hoekzema K, Fenckova M, Kvarnung M, Gerdts J, Trinh S, Cosemans N, Vives L, Lin J, Turner T N, Santen G, Ruivenkamp C, Kriek M, van Haeringen A, Aten E, Friend K, Liebelt J, Barnett C, Haan E, Shaw M, Gecz J, Anderlid B M, Nordgren A, Lindstrand A, Schwartz C, Kooy R F, Vandeweyer G, Helsmoortel C, Romano C, Alberti A, Vinci M, Avola E, Giusto S, Courchesne E, Pramparo T, Pierce K, Nalabolu S, Amaral D G, Scheffer I E, Delatycki M B, Lockhart P J, Hormozdiari F, Harich B, Castells-Nobau A, Xia K, Peeters H, Nordenskj old M, Schenck A, Bernier R A, Eichler E E. Targeted sequencing identifies 91 neurodevelopmental-disorder risk genes with autism and developmental-disability biases. Nat Genet. 2017; 49(4):515-26. Epub 2017/02/14. doi: 10.1038/ng.3792. PubMed PMID: 28191889; PMCID: PMC5374041.
2. van Bon B W, Hoischen A, Hehir-Kwa J, de Brouwer A P, Ruivenkamp C, Gijsbers A C, Marcelis C L, de Leeuw N, Veltman J A, Brunner H G, de Vries B B. Intragenic deletion in DYRK1A leads to mental retardation and primary microcephaly. Clin Genet. 2011; 79(3):296-9. Epub 2011/02/08. doi: 10.1111/j.1399-0004.2010.01544.x. PubMed PMID: 21294719.
3. Moller R S, Kubart S, Hoeltzenbein M, Heye B, Vogel I, Hansen C P, Menzel C, Ullmann R, Tommerup N, Ropers H H, Tumer Z, Kalscheuer V M. Truncation of the Down syndrome candidate gene DYRK1A in two unrelated patients with microcephaly. Am J Hum Genet. 2008; 82(5):1165-70. Epub 2008/04/15. doi: 10.1016/j.ajhg.2008.03.001. PubMed PMID: 18405873; PMCID: PMC2427221.
4. Luco S M, Pohl D, Sell E, Wagner J D, Dyment D A, Daoud H. Case report of novel DYRK1A mutations in 2 individuals with syndromic intellectual disability and a review of the literature. BMC Med Genet. 2016; 17:15. Epub 2016/02/29. doi: 10.1186/s12881-016-0276-4. PubMed PMID: 26922654; PMCID: PMC4769499.
5. Ji J, Lee H, Argiropoulos B, Dorrani N, Mann J, Martinez-Agosto J A, Gomez-Ospina N, Gallant N, Bernstein J A, Hudgins L, Slattery L, Isidor B, Le Caignec C, David A, Obersztyn E, Wisniowiecka-Kowalnik B, Fox M, Deignan J L, Vilain E, Hendricks E, Horton Harr M, Noon S E, Jackson J R, Wilkens A, Mirzaa G, Salamon N, Abramson J, Zackai E H, Krantz I, Innes A M,
Nelson S F, Grody W W, Quintero-Rivera F. DYRK1A haploinsufficiency causes a new recognizable syndrome with microcephaly, intellectual disability, speech impairment, and distinct facies. Eur J Hum Genet. 2015; 23(11):1473-81. Epub 2015/05/07. doi: 10.1038/ejhg.2015.71. PubMed PMID: 25944381; PMCID: PMC4613469.
6. Earl R K, Turner T N, Mefford H C, Hudac C M, Gerdts J, Eichler E E, Bernier R A. Clinical phenotype of ASD-associated DYRK1A haploinsufficiency. Mol Autism. 2017; 8:54. Epub 2017/10/17. doi: 10.1186/s13229-017-0173-5. PubMed PMID: 29034068; PMCID: PMC5629761.
7. Becker W, Sippl W. Activation, regulation, and inhibition of DYRK1A. FEBS J. 2011; 278(2):246-56. Epub 2010/12/04. doi: 10.1111/j.1742-4658.2010.07956.x. PubMed PMID: 21126318.
8. Song W J, Song E A, Jung M S, Choi S H, Baik H H, Jin B K, Kim J H, Chung S H. Phosphorylation and inactivation of glycogen synthase kinase 3beta (GSK3beta) by dual-specificity tyrosine phosphorylation-regulated kinase 1A (DyrklA). J Biol Chem. 2015; 290(4):2321-33. Epub 2014/12/06. doi: 10.1074/jbc.M114.594952. PubMed PMID: 25477508; PMCID: PMC4303684.
9. Najas S, Arranz J, Lochhead P A, Ashford A L, Oxley D, Delabar J M, Cook S J, Barallobre M J, Arbones M L. DYRK1A-mediated Cyclin D1 Degradation in Neural Stem Cells Contributes to the Neurogenic Cortical Defects in Down Syndrome. EBioMedicine. 2015; 2(2):120-34. Epub 2015/07/03. doi: 10.1016/j.ebiom.2015.01.010. PubMed PMID: 26137553; PMCID: PMC4484814.
10. Woods Y L, Rena G, Morrice N, Barthel A, Becker W, Guo S, Unterman T G, Cohen P. The kinase DYRK1A phosphorylates the transcription factor FKHR at Ser329 in vitro, a novel in vivo phosphorylation site. Biochem J. 2001; 355(Pt 3):597-607. Epub 2001/04/20. PubMed PMID: 11311120; PMCID: PMC1221773.
11. Hammerle B, Ulin E, Guimera J, Becker W, Guillemot F, Tejedor F J. Transient expression of Mnb/Dyrk1a couples cell cycle exit and differentiation of neuronal precursors by inducing p27KIP1 expression and suppressing NOTCH signaling. Development. 2011; 138(12):2543-54. Epub 2011/05/26. doi: 10.1242/dev.066167. PubMed PMID: 21610031; PMCID: PMC3100710.
12. Park J, Oh Y, Yoo L, Jung M S, Song W J, Lee S H, Seo H, Chung K C. DyrklA phosphorylates p53 and inhibits proliferation of embryonic neuronal cells. J Biol Chem. 2010; 285(41):31895-906. Epub 2010/08/11. doi: 10.1074/jbc.M110.147520. PubMed PMID: 20696760; PMCID: PMC2951261.
13. Fotaki V, Dierssen M, Alcantara S, Martinez S, Marti E, Casas C, Visa J, Soriano E, Estivill X, Arbones M L. DyrklA haploinsufficiency affects viability and causes developmental delay and abnormal brain morphology in mice. Mol Cell Biol. 2002; 22(18):6636-47. Epub 2002/08/23. PubMed PMID: 12192061; PMCID: PMC135639.
14. Argue G, de Lagran M M, Arbones M L, Dierssen M. Age-associated motor and visuo-spatial learning phenotype in DyrklA heterozygous mutant mice. Neurobiol Dis. 2009; 36(2):312-9. Epub 2009/08/08. doi: 10.1016/j.nbd.2009.07.027. PubMed PMID: 19660545.
15. Argue G, Fotaki V, Fernandez D, Martinez de Lagran M, Arbones M L, Dierssen M. Impaired spatial learning strategies and novel object recognition in mice haploinsufficient for the dual specificity tyrosine-regulated kinase-1A (DyrklA). PLoS One. 2008; 3 (7): e2575. Epub 2008/07/24. doi: 10.1371/j ournal.pone.0002575. PubMed PMID: 18648535; PMCID: PMC2481280.
16. Dang T, Duan W Y, Yu B, Tong D L, Cheng C, Zhang Y F, Wu W, Ye K, Zhang W X, Wu M, Wu B B, An Y, Qiu Z L, Wu B L. Autism-associated Dyrk1a truncation mutants impair neuronal dendritic and spine growth and interfere with postnatal cortical development. Mol Psychiatry. 2018; 23(3):747-58. Epub 2017/02/09. doi: 10.1038/mp.2016.253. PubMed PMID: 28167836; PMCID: PMC5822466.
17. Franklin A V, King M K, Palomo V, Martinez A, McMahon L L, Jope R S. Glycogen synthase kinase-3 inhibitors reverse deficits in long-term potentiation and cognition in fragile X mice. Biol Psychiatry. 2014; 75(3):198-206. Epub 2013/09/18. doi: 10.1016/j.biopsych.2013.08.003. PubMed PMID: 24041505; PMCID: PMC3874248.
18. Mines M A, Yuskaitis C J, King M K, Beurel E, Jope R S. GSK3 influences social preference and anxiety-related behaviors during social interaction in a mouse model of fragile X syndrome and autism. PLoS One. 2010; 5 (3): e9706. Epub 2010/03/20. doi: 10.1371/journal.pone.0009706. PubMed PMID: 20300527; PMCID: PMC2838793.
19. Chen Y, Huang W C, Sejourne J, Clipperton-Allen A E, Page D T. Pten Mutations Alter Brain Growth Trajectory and Allocation of Cell Types through Elevated beta-Catenin Signaling. J Neurosci. 2015; 35(28):10252-67. Epub 2015/07/17. doi: 10.1523/JNEUROSCI.5272-14.2015. PubMed PMID: 26180201.
20. Huang W C, Chen Y, Page D T. Hyperconnectivity of prefrontal cortex to amygdala projections in a mouse model of macrocephaly/autism syndrome. Nat Commun. 2016; 7: 13421. Epub 2016/11/16. doi: 10.1038/ncomms13421. PubMed PMID: 27845329; PMCID: PMC5116076 maps and institutional affiliations.
21. Vithayathil J, Pucilowska J, Landreth G E. ERK/MAPK signaling and autism spectrum disorders. Prog Brain Res. 2018; 241:63-112. Epub 2018/11/19. doi: 10.1016/bs.pbr.2018.09.008. PubMed PMID: 30447757.
22. Kim W Y, Wang X, Wu Y, Doble B W, Patel S, Woodgett J R, Snider W D. GSK-3 is a master regulator of neural progenitor homeostasis. Nat Neurosci. 2009; 12(11):1390-7. Epub 2009/10/06. doi: 10.1038/nn.2408. PubMed PMID: 19801986; PMCID: PMC5328673.
23. Cloetta D, Thomanetz V, Baranek C, Lustenberger R M, Lin S, Oliveri F, Atanasoski S, Ruegg M A. Inactivation of mTORC1 in the developing brain causes microcephaly and affects gliogenesis. J Neurosci. 2013; 33(18):7799-810. Epub 2013/05/03. doi: 10.1523/JNEUROSCI.3294-12.2013. PubMed PMID: 23637172; PMCID: PMC6618947.
24. Pucilowska J, Puzerey P A, Karlo J C, Galan R F, Landreth G E. Disrupted ERK signaling during cortical development leads to abnormal progenitor proliferation, neuronal and network excitability and behavior, modeling human neuro-cardio-facial-cutaneous and related syndromes. J Neurosci. 2012; 32(25):8663-77. Epub 2012/06/23. doi: 10.1523/JNEUROSCI.1107-12.2012. PubMed PMID: 22723706; PMCID: PMC6620980.
25. Caracci M O, Avila M E, De Ferrari G V. Synaptic Wnt/GSK3beta Signaling Hub in Autism. Neural Plast. 2016; 2016: 9603751. Epub 2016/02/18. doi: 10.1155/2016/9603751. PubMed PMID: 26881141; PMCID: PMC4736967.
26. Winden K D, Ebrahimi-Fakhari D, Sahin M. Abnormal mTOR Activation in Autism. Annu Rev Neurosci. 2018; 41:1-23. Epub 2018/03/01. doi: 10.1146/annurev-neuro-080317-061747. PubMed PMID: 29490194.
27. Ka M, Condorelli G, Woodgett J R, Kim W Y. mTOR regulates brain morphogenesis by mediating GSK3 signaling. Development. 2014; 141(21):4076-86. Epub 2014/10/03. doi: 10.1242/dev.108282. PubMed PMID: 25273085; PMCID: PMC4302893.
28. Lipton J O, Sahin M. The neurology of mTOR. Neuron. 2014; 84(2):275-91. Epub 2014/11/07. doi: 10.1016/j.neuron.2014.09.034. PubMed PMID: 25374355; PMCID: PMC4223653.
29. Gazestani V H, Pramparo T, Nalabolu S, Kellman B P, Murray S, Lopez L, Pierce K, Courchesne E, Lewis N E. A perturbed gene network containing PI3K-AKT, RAS-ERK and WNT-beta-catenin pathways in leukocytes is linked to ASD genetics and symptom severity. Nat Neurosci. 2019; 22(10):1624-34. Epub 2019/09/26. doi: 10.1038/s41593-019-0489-x. PubMed PMID: 31551593; PMCID: PMC6764590.
30. Kelly P A, Rahmani Z. DYRK1A enhances the mitogen-activated protein kinase cascade in PC12 cells by forming a complex with Ras, B-Raf, and MEK1. Mol Biol Cell. 2005; 16(8):3562-73. Epub 2005/05/27. doi: 10.1091/mbc.e04-12-1085. PubMed PMID: 15917294; PMCID: PMC1182298.
31. Abekhoukh S, Planque C, Ripoll C, Urbaniak P, Paul J L, Delabar J M, Janel N. DyrklA, a serine/threonine kinase, is involved in ERK and Akt activation in the brain of hyperhomocysteinemic mice. Mol Neurobiol. 2013; 47(1):105-16. Epub 2012/08/28. doi: 10.1007/s12035-012-8326-1. PubMed PMID: 22923366.
32. Woods Y L, Cohen P, Becker W, Jakes R, Goedert M, Wang X, Proud C G. The kinase DYRK phosphorylates protein-synthesis initiation factor eIF2Bepsilon at Ser539 and the microtubule-associated protein tau at Thr212: potential role for DYRK as a glycogen synthase kinase 3-priming kinase. Biochem J. 2001; 355 (Pt 3):609-15. Epub 2001/04/20. doi: 10.1042/bj3550609. PubMed PMID: 11311121; PMCID: PMC1221774.
33. Perluigi M, Pupo G, Tramutola A, Cini C, Coccia R, Barone E, Head E, Butterfield D A, Di Domenico F. Neuropathological role of PI3K/Akt/mTOR axis in Down syndrome brain. Biochim Biophys Acta. 2014; 1842(7):1144-53. Epub 2014/04/17. doi: 10.1016/j.bbadis.2014.04.007. PubMed PMID: 24735980; PMCID: PMC4062876.
34. Gorski J A, Talley T, Qiu M, Puelles L, Rubenstein J L, Jones K R. Cortical excitatory neurons and glia, but not GABAergic neurons, are produced in the Emx1-expressing lineage. J Neurosci. 2002; 22(15):6309-14. Epub 2002/08/02. doi: 20026564. PubMed PMID: 12151506; PMCID: PMC6758181.
35. Herculano-Houzel S, von Bartheld C S, Miller D J, Kaas J H. How to count cells: the advantages and disadvantages of the isotropic fractionator compared with stereology. Cell Tissue Res. 2015; 360(1):29-42. Epub 2015/03/06. doi: 10.1007/s00441-015-2127-6. PubMed PMID: 25740200; PMCID: PMC4380666.
36. Saunders A, Macosko E Z, Wysoker A, Goldman M, Krienen F M, de Rivera H, Bien E, Baum M, Bortolin L, Wang S, Goeva A, Nemesh J, Kamitaki N, Brumbaugh S, Kulp D, McCarroll S A. Molecular Diversity and Specializations among the Cells of the Adult Mouse Brain. Cell. 2018; 174(4):1015-30 e16. Epub 2018/08/11. doi: 10.1016/j.cell.2018.07.028. PubMed PMID: 30096299; PMCID: PMC6447408.
37. Hammerle B, Elizalde C, Tejedor F J. The spatio-temporal and subcellular expression of the candidate Down syndrome gene Mnb/DyrklA in the developing mouse brain suggests distinct sequential roles in neuronal development. Eur J Neurosci. 2008; 27(5):1061-74. Epub 2008/03/28. doi: 10.1111/j.1460-9568.2008.06092.x. PubMed PMID: 18364031.
38. Ko J. Neuroanatomical Substrates of Rodent Social Behavior: The Medial Prefrontal Cortex and Its Projection Patterns. Front Neural Circuits. 2017; 11:41. Epub 2017/07/01. doi: 10.3389/fncir.2017.00041. PubMed PMID: 28659766; PMCID: PMC5468389.
39. Canty A J, Murphy M. Molecular mechanisms of axon guidance in the developing corticospinal tract. Prog Neurobiol. 2008; 85(2):214-35. Epub 2008/04/02. doi: 10.1016/j.pneurobio.2008.02.001. PubMed PMID: 18378059.
40. Ryoo S R, Jeong H K, Radnaabazar C, Yoo J J, Cho H J, Lee H W, Kim I S, Cheon Y H, Ahn Y S, Chung S H, Song W J. DYRK1A-mediated hyperphosphorylation of Tau. A functional link between Down syndrome and Alzheimer disease. J Biol Chem. 2007; 282(48):34850-7. Epub 2007/10/02. doi: 10.1074/jbc.M707358200. PubMed PMID: 17906291.
41. Huang E J, Reichardt L F. Trk receptors: roles in neuronal signal transduction. Annu Rev Biochem. 2003; 72:609-42. Epub 2003/04/05. doi: 10.1146/annurev.biochem.72.121801.161629. PubMed PMID: 12676795.
42. Slipczuk L, Bekinschtein P, Katche C, Cammarota M, Izquierdo I, Medina J H. BDNF activates mTOR to regulate GluR1 expression required for memory formation. PLoS One. 2009; 4 (6): e6007. Epub 2009/06/24. doi: 10.1371/journal.pone.0006007. PubMed PMID: 19547753; PMCID: PMC2695538.
43. Carracedo A, Pandolfi P P. The PTEN-PI3K pathway: of feedbacks and cross-talks. Oncogene. 2008; 27(41):5527-41. Epub 2008/09/17. doi: 10.1038/onc.2008.247. PubMed PMID: 18794886.
44. Sara V R, Carlsson-Skwirut C, Bergman T, Jornvall H, Roberts P J, Crawford M, Hakansson L N, Civalero I, Nordberg A. Identification of Gly-Pro-Glu (GPE), the aminoterminal tripeptide of insulin-like growth factor 1 which is truncated in brain, as a novel neuroactive peptide. Biochem Biophys Res Commun. 1989; 165(2):766-71. Epub 1989/12/15. doi: 10.1016/50006-291x(89)80032-4. PubMed PMID: 2574573.
45. Tropea D, Giacometti E, Wilson N R, Beard C, McCurry C, Fu D D, Flannery R, Jaenisch R, Sur M. Partial reversal of Rett Syndrome-like symptoms in MeCP2 mutant mice. Proc Natl Acad Sci USA. 2009; 106(6):2029-34. Epub 2009/02/12. doi: 10.1073/pnas.0812394106. PubMed PMID: 19208815; PMCID: PMC2644158.
46. Arranz J, Balducci E, Arato K, Sanchez-Elexpuru G, Najas S, Parras A, Rebollo E, Pijuan I, Erb I, Verde G, Sahun I, Barallobre M J, Lucas J J, Sanchez M P, de la Luna S, Arbones M L. Impaired development of neocortical circuits contributes to the neurological alterations in DYRK1A haploinsufficiency syndrome. Neurobiol Dis. 2019; 127:210-22. Epub 2019/03/05. doi: 10.1016/j.nbd.2019.02.022. PubMed PMID: 30831192.
47. Souchet B, Guedj F, Sahun I, Duchon A, Daubigney F, Badel A, Yanagawa Y, Barallobre M J, Dierssen M, Yu E, Herault Y, Arbones M, Janel N, Creau N, Delabar J M. Excitation/inhibition balance and learning are modified by Dyrk1a gene dosage. Neurobiol Dis. 2014; 69:65-75. Epub 2014/05/08. doi: 10.1016/j.nbd.2014.04.016. PubMed PMID: 24801365.
48. Tejedor F, Zhu X R, Kaltenbach E, Ackermann A, Baumann A, Canal I, Heisenberg M, Fischbach K F, Pongs 0. minibrain: a new protein kinase family involved in postembryonic neurogenesis in Drosophila. Neuron. 1995; 14(2):287-301. Epub 1995/02/01. doi: 10.1016/0896-6273(95)90286-4. PubMed PMID: 7857639.
49. Hammerle B, Vera-Samper E, Speicher S, Arencibia R, Martinez S, Tejedor F J. Mnb/DyrklA is transiently expressed and asymmetrically segregated in neural progenitor cells at the transition to neurogenic divisions. Dev Biol. 2002; 246(2):259-73. Epub 2002/06/08. doi: 10.1006/dbio.2002.0675. PubMed PMID: 12051815.
50. Yabut O, Domogauer J, D'Arcangelo G. Dyrk1A overexpression inhibits proliferation and induces premature neuronal differentiation of neural progenitor cells. J Neurosci. 2010; 30(11):4004-14. Epub 2010/03/20. doi: 10.1523/JNEUROSCI.4711-09.2010. PubMed PMID: 20237271; PMCID: PMC3842457.
51. Spruston N. Pyramidal neurons: dendritic structure and synaptic integration. Nat Rev Neurosci. 2008; 9(3):206-21. Epub 2008/02/14. doi: 10.1038/nrn2286. PubMed PMID: 18270515.
52. Cheriyan J, Kaushik M K, Ferreira A N, Sheets P L. Specific Targeting of the Basolateral Amygdala to Projectionally Defined Pyramidal Neurons in Prelimbic and Infralimbic Cortex. eNeuro. 2016; 3 (2). Epub 2016/03/30. doi: 10.1523/ENEUR0.0002-16.2016. PubMed PMID: 27022632; PMCID: PMC4804386.
53. Adolphs R. Cognitive neuroscience of human social behaviour. Nat Rev Neurosci. 2003; 4(3):165-78. Epub 2003/03/04. doi: 10.1038/nrn1056. PubMed PMID: 12612630.
54. Ori-McKenney K M, McKenney R J, Huang H H, Li T, Meltzer S, Jan L Y, Vale R D, Wiita A P, Jan Y N. Phosphorylation of beta-Tubulin by the Down Syndrome Kinase, Minibrain/DYRK1a, Regulates Microtubule Dynamics and Dendrite Morphogenesis. Neuron. 2016; 90(3):551-63. Epub 2016/04/27. doi: 10.1016/j.neuron.2016.03.027. PubMed PMID: 27112495; PMCID: PMC4860041.
55. Xu B, Zang K, Ruff N L, Zhang Y A, McConnell S K, Stryker M P, Reichardt L F. Cortical degeneration in the absence of neurotrophin signaling: dendritic retraction and neuronal loss after removal of the receptor TrkB. Neuron. 2000; 26(1):233-45. Epub 2000/05/08. doi: 10.1016/s0896-6273(00)81153-8. PubMed PMID: 10798407.
56. Minichiello L. TrkB signalling pathways in LTP and learning. Nat Rev Neurosci. 2009; 10(12):850-60. Epub 2009/11/21. doi: 10.1038/nrn2738. PubMed PMID: 19927149.
57. Takei N, Nawa H. mTOR signaling and its roles in normal and abnormal brain development. Front Mol Neurosci. 2014; 7:28. Epub 2014/05/06. doi: 10.3389/fnmo1.2014.00028. PubMed PMID: 24795562; PMCID: PMC4005960.
58. Kelleher R J, 3rd, Bear M F. The autistic neuron: troubled translation? Cell. 2008; 135(3):401-6. Epub 2008/11/06. doi: 10.1016/j.cell.2008.10.017. PubMed PMID: 18984149.
59. Bozdagi O, Tavassoli T, Buxbaum J D. Insulin-like growth factor-1 rescues synaptic and motor deficits in a mouse model of autism and developmental delay. Mol Autism. 2013; 4(1):9. Epub 2013/04/30. doi: 10.1186/2040-2392-4-9. PubMed PMID: 23621888; PMCID: PMC3649942.
60. Castro J, Garcia R I, Kwok S, Banerjee A, Petravicz J, Woodson J, Mellios N, Tropea D, Sur M. Functional recovery with recombinant human IGF1 treatment in a mouse model of Rett Syndrome. Proc Natl Acad Sci USA. 2014; 111(27):9941-6. Epub 2014/06/25. doi: 10.1073/pnas.1311685111. PubMed PMID: 24958891; PMCID: PMC4103342.
61. Beck K D, Powell-Braxton L, Widmer H R, Valverde J, Hefti F. Igfl gene disruption results in reduced brain size, CNS hypomyelination, and loss of hippocampal granule and striatal parvalbumin-containing neurons. Neuron. 1995; 14(4):717-30. Epub 1995/04/01. doi: 10.1016/0896-6273(95)90216-3. PubMed PMID: 7718235.
62. Rangasamy S, Olfers S, Gerald B, Hilbert A, Svej da S, Narayanan V. Reduced neuronal size and mTOR pathway activity in the Mecp2 A140V Rett syndrome mouse model. F1000Res. 2016; 5:2269. Epub 2016/10/27. doi: 10.12688/f1000research.8156.1. PubMed PMID: 27781091; PMCID: PMC5040159.
63. Switon K, Kotulska K, Janusz-Kaminska A, Zmorzynska J, Jaworski J. Molecular neurobiology of mTOR. Neuroscience. 2017; 341:112-53. Epub 2016/11/28. doi: 10.1016/j.neuroscience.2016.11.017. PubMed PMID: 27889578.
1A. H. A. Stessman et al., Targeted sequencing identifies 91 neurodevelopmental-disorder risk genes with autism and developmental-disability biases. Nat Genet 49, 515-526 (2017).
2A. R. K. Earl et al., Clinical phenotype of ASD-associated DYRK1A haploinsufficiency. Mol Autism 8, 54 (2017).
3A. J. Ji et al., DYRK1A haploinsufficiency causes a new recognizable syndrome with microcephaly, intellectual disability, speech impairment, and distinct facies. Eur J Hum Genet 23, 1473-1481 (2015).
4A. R. S. Moller et al., Truncation of the Down syndrome candidate gene DYRK1A in two unrelated patients with microcephaly. Am J Hum Genet 82, 1165-1170 (2008).
5A. B. W. van Bon et al., Disruptive de novo mutations of DYRK1A lead to a syndromic form of autism and I D. Mol Psychiatry 21, 126-132 (2016).
6A. B. W. van Bon et al., Intragenic deletion in DYRK1A leads to mental retardation and primary microcephaly. Clin Genet 79, 296-299 (2011).
7A. J. A. Levy, C. W. LaFlamme, G. Tsaprailis, G. Crynen, D. T. Page, Dyrk1a Mutations Cause Undergrowth of Cortical Pyramidal Neurons via Dysregulated Growth Factor Signaling. Biol Psychiatry 90, 295-306 (2021).
8A. J. Park et al., Dyrk1A phosphorylates p53 and inhibits proliferation of embryonic neuronal cells. J Blot Chem 285, 31895-31906 (2010).
9A. W. J. Song et al., Phosphorylation and inactivation of glycogen synthase kinase 3beta (GSK3beta) by dual-specificity tyrosine phosphorylation-regulated kinase 1A (Dyrk1A). J Biol Chem 290, 2321-2333 (2015).
10A. B. Hammerle et al., Transient expression of Mnb/Dyrk1a couples cell cycle exit and differentiation of neuronal precursors by inducing p27KIP1 expression and suppressing NOTCH signaling. Development 138, 2543-2554 (2011).
11A. M. N. Shaikh, F. J. Tejedor, Mnb/DyrklA orchestrates a transcriptional network at the transition from self-renewing neurogenic progenitors to postmitotic neuronal precursors. J Neurogenet 32, 37-50 (2018).
12A. Y. L. Woods et al., The kinase DYRK1A phosphorylates the transcription factor FKHR at Ser329 in vitro, a novel in vivo phosphorylation site. Biochem J 355, 597-607 (2001).
13A. Y. L. Woods et al., The kinase DYRK phosphorylates protein-synthesis initiation factor eIF2Bepsilon at Ser539 and the microtubule-associated protein tau at Thr212: potential role for DYRK as a glycogen synthase kinase 3-priming kinase. Biochem J 355, 609-615 (2001).
14A. T. M. Scales, S. Lin, M. Kraus, R. G. Goold, P. R. Gordon-Weeks, Nonprimed and DYRK1A-primed GSK3 beta-phosphorylation sites on MAP1B regulate microtubule dynamics in growing axons. J Cell Sci 122, 2424-2435 (2009).
15A. N. Kurabayashi, T. Hirota, M. Sakai, K. Sanada, Y. Fukada, DYRK1A and glycogen synthase kinase 3beta, a dual-kinase mechanism directing proteasomal degradation of CRY2 for circadian timekeeping. Mol Cell Biol 30, 1757-1768 (2010).
16A. M. S. Jung et al., Regulation of RCAN1 protein activity by DyrklA protein-mediated phosphorylation. J Blot Chem 286, 40401-40412 (2011).
17A. S. Abekhoukh et al., Dyrk1A, a serine/threonine kinase, is involved in ERK and Akt activation in the brain of hyperhomocysteinemic mice. Mol Neurobiol 47, 105-116 (2013).
18A. A. M. Iyer et al., mTOR Hyperactivation in down syndrome hippocampus appears early during development. J Neuropathol Exp Neurol 73, 671-683 (2014).
19A. M. Perluigi et al., Neuropathological role of PI3K/Akt/mTOR axis in Down syndrome brain. Biochim Biophys Acta 1842, 1144-1153 (2014).
20A. X. M. Ma, J. Blenis, Molecular mechanisms of mTOR-mediated translational control. Nat Rev Mol Cell Biol 10, 307-318 (2009).
21A. M. E. Cardenas, N. S. Cutler, M. C. Lorenz, C. J. Di Como, J. Heitman, The TOR signaling cascade regulates gene expression in response to nutrients. Genes Dev 13, 3271-3279 (1999).
22A. T. Powers, P. Walter, Regulation of ribosome biogenesis by the rapamycin-sensitive TOR-signaling pathway in Saccharomyces cerevisiae. Mol Blot Cell 10, 987-1000 (1999). 23A. N. C. Barbet et al., TOR controls translation initiation and early G1 progression in yeast. Mol Blot Cell 7, 25-42 (1996).
24A. M. H. Lu, Y. P. Hsueh, Protein synthesis as a modifiable target for autism-related dendritic spine pathophysiologies. FEBS J, (2021).
25A. L. H. Lo, K. O. Lai, Dysregulation of protein synthesis and dendritic spine morphogenesis in ASD: studies in human pluripotent stem cells. Mol Autism 11, 40 (2020).
26A. Z. X. Xu et al., Elevated protein synthesis in microglia causes autism-like synaptic and behavioral aberrations. Nat Commun 11, 1797 (2020).
27A. W. C. Huang, Y. Chen, D. T. Page, Hyperconnectivity of prefrontal cortex to amygdala projections in a mouse model of macrocephaly/autism syndrome. Nat Commun 7, 13421 (2016).
28A. R. J. Kelleher, 3rd, M. F. Bear, The autistic neuron: troubled translation? Cell 135, 401-406 (2008).
29A. J. J. LeBlanc, M. Fagiolini, Autism: a “critical period” disorder? Neural Plast 2011, 921680 (2011).
30A. P. Bang, M. Polak, J. Woelfle, A. Houchard, E. I. R. S. Group, Effectiveness and Safety of rhIGF-1 Therapy in Children: The European Increlex® Growth Forum Database Experience. Horm Res Paediatr 83, 345-357 (2015).
31A. J. Castro et al., Functional recovery with recombinant human IGF1 treatment in a mouse model of Rett Syndrome. Proc Natl Acad Sci USA 111, 9941-9946 (2014).
32A. D. Tropea et al., Partial reversal of Rett Syndrome-like symptoms in MeCP2 mutant mice. Proc Natl Acad Sci USA 106, 2029-2034 (2009).
33A. J. Zhou et al., Pharmacological inhibition of mTORC1 suppresses anatomical, cellular, and behavioral abnormalities in neural-specific Pten knock-out mice. J Neurosci 29, 1773-1783 (2009).
34A. S. Himpel et al., Specificity determinants of substrate recognition by the protein kinase DYRK1A. J Biol Chem 275, 2431-2438 (2000).
35A. H. L. Rust, P. R. Thompson, Kinase consensus sequences: a breeding ground for crosstalk. ACS Chem Biol 6, 881-892 (2011).
36A. M. Soundararaj an et al., Structures of Down syndrome kinases, DYRKs, reveal mechanisms of kinase activation and substrate recognition. Structure 21, 986-996 (2013).
37A. A. Recasens et al., Global phosphoproteomics reveals DYRK1A regulates CDK1 activity in glioblastoma cells. Cell Death Discov 7, 81 (2021).
38A. F. Gnad, J. Gunawardena, M. Mann, PHOSIDA 2011: the posttranslational modification database. Nucleic Acids Res 39, D253-260 (2011).
39A. F. Gnad et al., PHOSIDA (phosphorylation site database): management, structural and evolutionary investigation, and prediction of phosphosites. Genome Biol 8, R250 (2007).
40A. K. Inoki et al., TSC2 integrates Wnt and energy signals via a coordinated phosphorylation by AMPK and GSK3 to regulate cell growth. Cell 126, 955-968 (2006).
41A. H. H. Zhang, A. I. Lipovsky, C. C. Dibble, M. Sahin, B. D. Manning, S6K1 regulates GSK3 under conditions of mTOR-dependent feedback inhibition of Akt. Mol Cell 24, 185-197 (2006).
42A. G. I. Welsh, C. M. Miller, A. J. Loughlin, N. T. Price, C. G. Proud, Regulation of eukaryotic initiation factor eIF2B: glycogen synthase kinase-3 phosphorylates a conserved serine which undergoes dephosphorylation in response to insulin. FEBS Lett 421, 125-130 (1998).
43A. E. M. Paronett, D. W. Meechan, B. A. Karpinski, A. S. LaMantia, T. M. Maynard, Ranbpl, Deleted in DiGeorge/22q11.2 Deletion Syndrome, is a Microcephaly Gene That Selectively Disrupts Layer 2/3 Cortical Projection Neuron Generation. Cereb Cortex 25, 3977-3993 (2015).
44A. G. D. Gilfillan et al., SLC9A6 mutations cause X-linked mental retardation, microcephaly, epilepsy, and ataxia, a phenotype mimicking Angelman syndrome. Am J Hum Genet 82, 1003-1010 (2008).
45A. E. D. Gamsiz et al., Discovery of Rare Mutations in Autism: Elucidating Neurodevelopmental Mechanisms. Neurotherapeutics 12, 553-571 (2015).
46A. D. D. Armstrong, Neuropathology of Rett syndrome. J Child Neurol 20, 747-753 (2005).
47A. C. A. Williams, D. J. Driscoll, A. I. Dagli, Clinical and genetic aspects of Angelman syndrome. Genet Med 12, 385-395 (2010).
48A. K. D. Winden, D. Ebrahimi-Fakhari, M. Sahin, Abnormal mTOR Activation in Autism. Annu Rev Neurosci 41, 1-23 (2018).
49A. B. L. Finlay, R. B. Darlington, Linked regularities in the development and evolution of mammalian brains. Science 268, 1578-1584 (1995).
50A. B. Crain, C. Cotman, D. Taylor, G. Lynch, A quantitative electron microscopic study of synaptogenesis in the dentate gyrus of the rat. Brain Res 63, 195-204 (1973).
51A. B. D. Semple, K. Blomgren, K. Gimlin, D. M. Ferriero, L. J. Noble-Haeusslein, Brain development in rodents and humans: Identifying benchmarks of maturation and vulnerability to injury across species. Prog Neurobiol 106-107, 1-16 (2013).
52A. R. L. Cox, F. Calderon de Anda, T. Mangoubi, A. Yoshii, Multiple Critical Periods for Rapamycin Treatment to Correct Structural Defects in Tsc-1-Suppressed Brain. Front Mol Neurosci 11, 409 (2018).
53A. M. van Lier et al., Disruption of Critical Period Plasticity in a Mouse Model of Neurofibromatosis Type 1. J Neurosci 40, 5495-5509 (2020).
54A. C. L. Gatto, K. Broadie, Temporal requirements of the fragile x mental retardation protein in modulating circadian clock circuit synaptic architecture. Front Neural Circuits 3, 8 (2009).
55A. C. L. Gatto, K. Broadie, Temporal requirements of the fragile X mental retardation protein in the regulation of synaptic structure. Development 135, 2637-2648 (2008).
56A. H. Deng et al., Inhibition of glycogen synthase kinase-3beta is sufficient for airway smooth muscle hypertrophy. J Blot Chem 283, 10198-10207 (2008).
57A. K. Spittaels et al., Neonatal neuronal overexpression of glycogen synthase kinase-3 beta reduces brain size in transgenic mice. Neuroscience 113, 797-808 (2002).

The invention thus has been disclosed broadly and illustrated in reference to representative embodiments described above. It is understood that various modifications can be made to the present invention without departing from the spirit and scope thereof.

It is further noted that all publications, patents and patent applications cited herein are hereby expressly incorporated by reference in their entirety and for all purposes as if each is individually so denoted. Definitions that are contained in text incorporated by reference are excluded to the extent that they contradict definitions in this disclosure.

1. A method of treating a DYRK1A-related disorder comprising administering IGF-1, (1-3)IGF-1, Trofinetide, NNZ-2591, a Gsk3β inhibitor, or a Pten inhibitor.

2. The method of claim 1, wherein the DYRK1A-related disorder comprises a DYRK1A syndrome, an autism spectrum disorder (ASD), intellectual disability (ID), microcephaly and sociability deficits.

3. The method of claim 1, wherein treating the DYRK1A-related disorder comprises improving sociability, decreasing microcephaly, increasing spine density, and/or improving synaptic function.

4. A method for increasing cortical mass, cortical cell growth, neuronal cell growth, protein synthesis, and/or spinal density in a subject having a loss of function DYRK1A mutation comprising administering IGF-1, (1-3)IGF-1, Trofinetide, NNZ-2591, or a Gsk3β inhibitor.

5. The method of claim 4 wherein the increase in cortical cell growth is an increase in cell size and/or cell number.

6. The method of claim 4 wherein the increase in neuronal cell growth is an increase in cell size and/or cell number.

7. The method of claim 1, comprising administering a Gsk3β inhibitor or a Pten inhibitor.

8-9. (canceled)

10. The method of claim 7, wherein the Gsk3β inhibitor comprises an antisense oligonucleotide or an siRNA wherein the antisense oligonucleotide or the siRNA targets the Gsk3β gene and causes a decrease in expression of the Gsk3β gene.

11. The method of claim 7, wherein the Gsk3β inhibitor comprises SB216763. SAR502250, indirubin-3-oxime, CHIR-99021, AT7519, TWS119, indirubin, SB415286, CHIR-98014, lithium, zinc, tungstate, an indirubin, 6-BIO, hymenialdisine, dibromocantharelline, a meridianin, an aminopyrimidine, CT98014, CT98023, CT99021, TWS119, an arylindolemaleimide, SB-41528, a thiazole, AR-A014418, AZD-1080, a paullone, kenpaullone, alsterpaullone, cazpaullone, an aloisine, a manzamine, manzamine A, a furanosesquiterpene, palinurine, tricantine, a thiadiazolidindione, TDZD-8, NP00111, NP031115, NP031112 (tideglusib), a halomethylketone, HMK-32, or L803-mts.

12. The method of claim 4, comprising administering a Gsk3β inhibitor.

13-14. (canceled)

15. A method of treating a DYRK1A-related disorder comprising administering BDNF, TrkB, mTOR, IGF-1 ERK1, or ERK2, nucleic acid encoding BDNF, TrkB, mTOR, IGF-1 ERK1, or ERK2, or a positive effector of BDNF, TrkB, mTOR, IGF-1 ERK1, or ERK2.

16. The method of claim 15, wherein the DYRK1A-related disorder comprises a DYRK1A syndrome, an autism spectrum disorder (ASD), intellectual disability (ID), microcephaly and sociability deficits.

17. The method of claim 15, wherein treating the DYRK1A-related disorder comprises improving sociability, decreasing microcephaly, increasing spine density, and/or improving synaptic function.

18. The method of claim 15, wherein the positive effector of BDNF comprises 7,8-dihydroxyflavone.

19. The method of claim 15, wherein the positive effector of TrkB comprises 7,8-dihydroxyflavone, deoxygedunin, LM22A-4, HIOC, R7, R13, AS86, Ab1A01, Ab104, Ab4B19, Ab2C03, Ab303, Ab1104, or Ab2908.

20. The method of claim 15 wherein the nucleic acid encoding BDNF, TrkB, mTOR, IGF-1 ERK1, or ERK2 comprises a plasmid, a viral vector, or an mRNA.

21-23. (canceled)

24. The method of claim 7, wherein the Pten inhibitor comprises an antisense oligonucleotide or an siRNA wherein the antisense oligonucleotide or the siRNA targets the PTen gene and causes a decrease in expression of the Pten gene.

25. The method of claim 7, wherein the Pten inhibitor comprises bpV(phen), bpV(pic), bpV(HOpic), bpV(pis), VO-OHpic, or SF1670.

26. The method of claim 1, comprising administering IGF-1, (1-3)IGF-1, Trofinetide, or NNZ-2591.

27. The method of claim 4, comprising administering IGF-1, (1-3)IGF-1, Trofinetide, or NNZ-2591.


SEQUENCE LISTING (134 sequences)

SEQ ID NO: 1 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 2 - DNA, 4766 bp, Homo sapiens
gcgcgcgcgcacacacacacacacacacagagagaacatctctagtaaaaagaaaagttgagctttcttagctagatgtgtgtattagccagaaaaagccaaggagtgaagggttttagagaactggaggagataaagtggagtctgcatatgggaggcatttgaaatggacttaaatgtctttttaatgctgactttttcagttttctccttaccagacacattgttttcatgacattagccccaggcatagacacatcattaaaatgaacatgtcaaaaaatgatttctgtttagaaataagcaaaacattttcagttgtgaccacccaggtgtagaataaagaacagtggaattgggagccctgagttctaacataaactttcttcatgacataaggcaagtcttctatggcctttggtttccttacctgtaaaacaggatggctcaatgaaattatctttcttctttgctataatagagtatctctgtgggaagaggaaaaaaaaagtcaatttaaaggctccttatagttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggtttatatgctagatcttgggggaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctgtatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaagcctattgtaaatacaagttctatttaagtgtactaattaacatataatatatgttttaaatatagaatttttaatgtttttaaatatattttcaaagtacataaaa

SEQ ID NO: 3 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 4 - DNA, 7491 bp, Homo sapiens
ggaaaaacagattccgagccgcaaaagggaagacggattctcagacaaggcttgcaaatgccccgcagccatcatttaactgcacccgcagaatagttacggtttgtcacccgaccctcccggatcgcctaatttgtccctagtgagaccccgaggctctgcccgcgcctggcttcttcgtagctggatgcatatcgtgctccgggcagcgcgggcgcagggcacgcgttcgcgcacaccctagcacacatgaacacgcgcaagggagacagcctctaccgcgattgtagaagagactgtggtgtgaattagggaccgggaggcgtcgaacggaggaacggttcatcttagagactaattttctggagtttctgcccctgctctgcgtcagccctcacgtcacttcgccagcagtagcagaggcggcggcggcggctcccggaattgggttggagcaggagcctcgctggctgcttcgctcgcgctctacgcgctcagtccccggcggtagcaggagcctggacccaggcgccgccggcgggcgtgaggcgccggagcccggcctcgaggtgcataccggacccccattcgcatctaacaaggaatctgcgccccagagagtcccgggagcgccgccggtcggtgcccggcgcgccgggccatgcagcgacggccgccgcggagctccgagcagcggtagcgcccccctgtaaagcggttcgctatgccggggccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggttttgttttgtttcataagatcccactggatgggtagctgaaataaaggaaaagacagagaaaggggctgtggtgcttgttggttgatgctgccatgtaagctggactcctgggactgctgttggcttatcccgggaagtgctgcttatctggggttttctggtagatgtgggcggtgtttggaggctgtactatatgaagcctgcatatactgtgagctgtgattggggaacaccaatgcagaggtaactctcaggcagctaagcagcacctcaagaaaacatgttaaattaatgcttctcttcttacagtagttcaaatacaaaactgaaatgaaatcccattggattgtacttctcttctgaaaagtgtgctttttgaccctactggacatttattgacttaattgcttctgtttattaaaattgacctgcaaagttaaaaaaaaattaaagttgagaacaggtataagtgcacactgaatagtctaatctacatgtaacacatattttagtgtgattttctatactctaatcagcactgaattcagagggtttgactttttcatctataacacagtgactaaaagagttaagggtatatataccatcactttgggacttggtagtattattaaaaggttatttccttcactgtcaataaaagtccaaatgtttagcttaggtctgagagtcaaacaatgttaaggattgtcttaaagttccttagccagcaaaacaaaacaaaacaaaacaaacaaatgaaaaacgtttaaaaagaagaagaagaaaaaaaacaagaacaagcagcaacagctgttttgttggggctatagatttaagttaggcatagtcaatttcagaataactaagagtggaatatatgcatatggtgaaattataaccttgcccttttttatttgccctctgcgatccacctgctttttagaagtctgccgagtgagaaggccacagtatctcatgctgtttgcattacagaactgcagcttttctactctgaaaaggcctgggagcagaatggctggcctgctgtgagcaggagaggagattctaagaaggatagtcccccctacaacatactgtcatactgctgggttttcatgggtaggaaagcttgtcctgaccccagcagcaaagaggtggcaggtcgctaatgaatatatgctttataatgtccttcttcattgctgagagggcagccttagagctgtggatttctgcatcccccctgagtctgacccatggacacctgtttcattcactttagcatcacagtgacctttgtatgctctgttcagtctgtgtcaggcagtatgcttgtcctgaagagaggtttggctatccccaccccaccccaccccaccctgttccttttttatcaggaggacttcagagccaggcctgcagcattttgtttgaaaacacaatcagctctgacagttagacatgcacacagacgccatagctggattggaaacattgatgttttaaaaatttattttttttggaaatagttgcacaaatgctgcaatttagctttaaggttctatagatttttaactagtccaacacagtcagaaacattgttttgaatcctctgtaaaccaaggcattaatcttaataaaccaggatccatttaggtaccacttgatataaaaaggatatccataatgaatattttatactgcatcctttacattagccactaaatacgttattgcttgatgaagacctttcacagaatcctatggattgcagcatttcacttggctacttcatacccatgccttaaagaggggcagtttctcaaaagcagaaacatgccgccagttctcaagttttcctcctaactccatttgaatgtaagggcagctggcccccaatgtggggaggtccgaacattttctgaattcccattttcttgttcgcggctaaatgacagtttctgtcattacttagattccgatctttcccaaaggtgttgatttacaaagaggccagctaatagcagaaatcatgaccctgaaagagagatgaaattcaagctgtgagccaggcaggagctcagtatggcaaaggttcttgagaatcagccatttggtacaaaaaagatttttaaagcttttatgttataccatggagccatagaaaggctatggattgtttaagaactattttaaagtgttccagacccaaaaaggaaaaataaaaaaaaaggaatatttgtacccaacagctagaaggattgcaaggtagatttttgttttaaaatggagagaagtggacagataaggccatttaatatatcaaagatcagttgacatctcctagggaatgatgaaaacagcaggctattagaaaattatttcatatagttctcgtgttcttttcttttttttaatccctgaagggatgatcagtaacatagcttctcttttctgtactctagaccaccccttttcatcattttgctttttatgtctcccataagaaatgtgctttttagagcttcctaatgcatgtgttgcattattgcagcattagaaaaggagaggtagcatttttgctgaaatcgggcctgtcactctccaataaaggttctggcacttcaatgccaggcaggtctcctaaatgaacagaatgatctgtgtgagccgatgcctgcccttccagaggggccactgtccccagccgcagccaactgtgtcccacaggaatgggagcctaggtttccaaatcttgtgattctttaggagaaacatgaaacctggatttcgtgtgaaatgtcccgattgttaaaaagttggctcaattatttttaaaacattttgtaagccaacaaaagtctgtgggctgccagtttattacttttgtcttaaaacatgatcattgttctctcacggtatccttctgtcttcccgttgcaaattcacttttctttcttcctgacattgccattgagggctttgttaccacaagctaagaaactgagtttaacagcccagttatctgcaacatgtcaattacctttgctcctctcctgtgattcccaccatgctgtgaccctcagctgtctccctttgctgggaattctgcaccaatgtctcccctcaacccattccctggttggtcctactcccgtgtggccagagacatcctagcaaatccttcctcctattatatctgacactaatttcttttcaacagcgctcatgtctcttggcccagtcaggtgctgccaggtttagataggaaagtacatgtcccattttcatgggtgcccttaatgtggtccacgtcctatatcttattatatttactcatggctcaatgggggcctccagagaccctctcaggctgctgagctagactaaggaatgcatccaccgtcatcacatgagacactgactctgtgacgacaaaagtacaaacagtctgaggctaagaaaggttcatctcacaacaggaaaaacaaatctcaacacacattagagataattgattcaggggttttctctcccagtctcccagcagggactgatttcatttctgacccactaggttttctttccagaaataggtagcaaggacaagaactaaacaatcccagccccacccagcaacacagaacacaggagtttgcttttggcttctcactctccaagtaaccctgaattaggcccagaatggctgaggcttggagcatctcctcagacagagcagaggcgacacctcttcaggggtgtgtggagtaaatagctcgaagagctgaagacagaaaaccagtttcacgccaggtgcgagagagagcataatggagggaagcccgctttctctctcctcttcttttctctttatttctttagagcacttgacttttttttctctctctctctagtattctaaactgaccccatgaccaactgagaatttatttttgtttcattggttgtttcacagaattagaacacacacgactttttattcctccattgcaaaatggaatcaagatactacacaagacctgtgctttcttcctttgcatgatttacacctccgcctgttttggtgctagctgtctagaacttctctcttggtttgaatctgattccttcacactacactagaagtttatttcatcttgttttgtctagactccagatacagagggacagctggactgaggacaagcaattccatctagcatagggtctctcagggttggtgcatccagccacatgggcagggccagtcacatctagtctatgtccccagagcccttggagttgcgcagcttagctgacttgactccaaggaaattagtacagaagtaaccactctattaagtgtgttctgctatgttcacatgcctgtagtacctgcaaaccatgccaggttcatctaaagacataggggaagattaaggactcttttggacagaccatgaattgaatttgctgccaggtgctgccagactgaatttggctgacagaactcccagcccaggaaagttccatgacaatgactgtcgcagaaggaaatttcccactaaagtcagtccattttcaagttttggtcttcagagacaaaagaacgtcccagccacctgattttgatggtgaggtaactctaagttgaattcaggctagtgttgcagtatagctttggcatgttcatgagtgagcacccagaatgtgttgaaccaacccccacccctaactactgactatgactgcagtgggtttttatggggaaaaaaagtgtgaaaagcaaaaagaaaggaacagagattttttatcacctttattgtaagacagtccatttatgaattgagtataaacacatacaaagtaacaagagattcctaagaaacgcaaatccttgagtttcacgcacttcatgttcaaccatttgctgtaatccagaggcagcctgtgaatcattctcatgccctgttttttttttttttttcctataatgttctgggtttaaaagccatcttttccacattttctgtaaataatggataatcattttaaaaatttttatttttagtgctgttttaacaatgtagatagatcataaatgtacttgctgaattcaatcatttttaacaagccaataaagtttgataattca

SEQ ID NO: 5 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 6 - DNA, 8721 bp, Homo sapiens
gctcccggcttagaggacagcggggaaggcgggcggtggggcagggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacata

SEQ ID NO: 7 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 8 - DNA, 7277 bp, Homo sapiens
cttctgtttgctaaatctcactgtcactgctaaattcagagcagatagagcctgcgcaatggaataaagtcctcaaaattgaaatgtgacattgctctcaacatctcccatctctctggatttctttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagattttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaacctcgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaagtttgttaaaatatgcttgactagagtttccagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacgtgttttctgtcatagttagatgtgataattctaagagtgtctatgacttatttccttcacttaattctatccacagtcaaaaatcccccaaggaggaaagctgaaagatgcactgccatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaacccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttccccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttgtttttgttttttttttttttttactttatacagtctttatgaatttcttaatgttcaaaatgacttggttcttttcttctttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctttttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggtctcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacattaaaattagtcatgtttgattaa

SEQ ID NO: 9 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 10 - DNA, 1085 bp, Homo sapiens
gaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgaggtgggccagtccccagcagcagtggggctgggggcaggggagcaggggggcacgtag

SEQ ID NO: 11 - protein, 360 aa, Homo sapiens [sequence omitted]

SEQ ID NO: 12 - DNA, 5881 bp, Homo sapiens
agtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 13 - protein, 29 aa, Homo sapiens
RETEALLQSIGISPEPPLVPTPMSPSSKS

SEQ ID NO: 14 - protein, 29 aa, Mus musculus
RETEALLQSIGISPEPPLVPTPMSPSSKS

SEQ ID NO: 15 - protein, 29 aa, Mus musculus
RETEALLQSIGISPEPPLVPTPMSPSSKS

SEQ ID NO: 16 - protein, 18 aa, Mus musculus
RSAESLQSLNSGLCPEKI

SEQ ID NO: 17 - protein, 17 aa, Mus musculus
KVASEEEEVPLVVYLKE

SEQ ID NO: 18 - protein, 38 aa, Mus musculus
KTLYEEAEEASDISQQVANLAISPTTPGPSWPGDALRL

SEQ ID NO: 19 - protein, 11 aa, Mus musculus
KASPVYLDILG

SEQ ID NO: 20 - protein, 24 aa, Mus musculus
KLDDGHLNNSLGSPVQADVYFPRL

SEQ ID NO: 21 - protein, 20 aa, Mus musculus
RSQGSPEDPPSQASPGSNKQ

SEQ ID NO: 22 - protein, 23 aa, Mus musculus
RASGQAFELILKPPSPISEAPRT

SEQ ID NO: 23 - protein, 23 aa, Mus musculus
KEAAGGNDSSGATSPINPAVALE

SEQ ID NO: 24 - protein, 25 aa, Mus musculus
KEKEAAGGNDSSGATSPINPAVALE

SEQ ID NO: 25 - protein, 16 aa, Mus musculus
RVPCNVEGISPELEKV

SEQ ID NO: 26 - protein, 34 aa, Mus musculus
RHLSNVSSTGSIDMVDSPQLATLADEVSASLAKQ

SEQ ID NO: 27 - protein, 34 aa, Mus musculus; identical to SEQ ID NO: 26

SEQ ID NO: 28 - protein, 34 aa, Mus musculus; identical to SEQ ID NO: 26

SEQ ID NO: 29 - protein, 15 aa, Mus musculus
MESPEGAGPGEIVKD

SEQ ID NO: 30 - protein, 28 aa, Mus musculus
RALYLEPSDGVSPTQETGEAQSQDDERS

SEQ ID NO: 31 - protein, 34 aa, Mus musculus
KQQESDDSLVDYGEGGEGQFNEDGSFIGQYTVKK

SEQ ID NO: 32 - protein, 21 aa, Mus musculus
RSEGVQDAEPQSLVGSPSTRG

SEQ ID NO: 33 - protein, 44 aa, Mus musculus
KAAATPEPAVAQPDSTALEGATGQAPPSSKGEEATGCAQESQRV

SEQ ID NO: 34 - protein, 29 aa, Mus musculus
KSEEETSPLVTHQNPAGPVAIAPELESKE

SEQ ID NO: 35 - protein, 37 aa, Mus musculus
KVEAIPNSKSEEETSPLVTHQNPAGPVAIAPELESKE

SEQ ID NO: 36 - protein, 23 aa, Mus musculus
RSPSGGAAGPLLTPSQSLDGSRR

SEQ ID NO: 37 - protein, 31 aa, Mus musculus
RSSLFPFEDAFLDDSHGDQALSSGLSSPTRC

SEQ ID NO: 38 - protein, 17 aa, Mus musculus
RNLASPEGTLATLGLKT

SEQ ID NO: 39 - protein, 19 aa, Mus musculus
RRISPADTPVSESSSPLKS

SEQ ID NO: 40 - protein, 22 aa, Mus musculus
RCLAFHDISPQAPTHFLVIPKK

SEQ ID NO: 41 - protein, 19 aa, Mus musculus
RLPATSAESESAVISNGEH

SEQ ID NO: 42 - protein, 18 aa, Mus musculus
RGNVLASHSPMSPENIKM

SEQ ID NO: 43 - protein, 26 aa, Mus musculus
KNMTVEQLLTGSPTSPTVEPEKPTRE

SEQ ID NO: 44 - protein, 23 aa, Mus musculus
KDGSPDAPATPEKEEVAFSEYKT

SEQ ID NO: 45 - protein, 40 aa, Mus musculus
KASQPSPPAQEAGYSTLAQSYTPDHPSELPEEPSSPQERM

SEQ ID NO: 46 - protein, 18 aa, Mus musculus
KETSPETSLIQDEVALKL

SEQ ID NO: 47 - protein, 43 aa, Mus musculus
KDQPAALPLAAEETANLPPSPPPSPASEQTATVEEDLLTASKM

SEQ ID NO: 48 - protein, 39 aa, Mus musculus
KEQPGNTVSSGQEDFPSVLFETAASLPSLSPLSTVSFKE

SEQ ID NO: 49 - protein, 36 aa, Mus musculus
KTSDSKEEIKEPESFNAAAQEAEAPYISIACDLIKE

SEQ ID NO: 50 - protein, 28 aa, Mus musculus
KERLSASPQEVGKPYLESFQPNLHITKD

SEQ ID NO: 51 - protein, 19 aa, Mus musculus
KSLVGTPYWMAPEVISRLS

SEQ ID NO: 52 - protein, 21 aa, Mus musculus
KQPGMGPGSPVSDVDQEEPRF

SEQ ID NO: 53 - protein, 14 aa, Mus musculus
KIGEGTYGVVYKAG

SEQ ID NO: 54 - protein, 21 aa, Mus musculus
RTPLATIADQQQLQLSPLKRL

SEQ ID NO: 55 - protein, 26 aa, Mus musculus
KLVGSGFPLPTSDLDDSLTEDIDEKI

SEQ ID NO: 56 - protein, 25 aa, Mus musculus
KKASEDESDLEDEEEKSQEDTEQKR

SEQ ID NO: 57 - protein, 26 aa, Mus musculus
KDGESYDPYDFSEAETQMPQVHTPKT

SEQ ID NO: 58 - protein, 18 aa, Mus musculus
KAVESTDEDHQTDLDAKM

SEQ ID NO: 59 - protein, 25 aa, Mus musculus
RAADEDWDSELEDDLLGEDLLSGKK

SEQ ID NO: 60 - protein, 18 aa, Mus musculus
RALSPLSPVAIEQTTLKM

SEQ ID NO: 61 - protein, 18 aa, Mus musculus
RALSPLSPVAIEQTTLKM

SEQ ID NO: 62 - protein, 15 aa, Mus musculus
KSSSQENLLDEVMKS

SEQ ID NO: 63 - protein, 30 aa, Mus musculus
KAALGLQDSDDEDAAVDIDEQIESMFNSKK

SEQ ID NO: 64 - protein, 20 aa, Mus musculus
KSISEGHPWHVPDSPSHSKH

SEQ ID NO: 65 - protein, 34 aa, Mus musculus
KILDPNSGEPAPVLSSPTPTDLSTFLSFPSPEKL

SEQ ID NO: 66 - protein, 20 aa, Mus musculus
RYLQDDTSDPTYTSSLGGKI

SEQ ID NO: 67 - protein, 16 aa, Mus musculus
RAQSGSPESSSGQTKG

SEQ ID NO: 68 - protein, 13 aa, Mus musculus
RGSPHYFSPFRPY

SEQ ID NO: 69 - protein, 18 aa, Mus musculus
KEELASDLEEMATSSAKR

SEQ ID NO: 70 - protein, 20 aa, Mus musculus
KEIITEEPSEEEADMPKPKK

SEQ ID NO: 71 - protein, 21 aa, Mus musculus
RVGTNYQAVIPECKPESPARY

SEQ ID NO: 72 - protein, 48 aa, Mus musculus
KESSTAAPSGAGNVASGSGNNSGGPSLLVPLPVNPPSSPTPSFSEAKA

SEQ ID NO: 73 - protein, 19 aa, Mus musculus
RILSDVTHSAVFGVPASKS

SEQ ID NO: 74 - protein, 25 aa, Mus musculus
RDKRPLSGPDVSTPQPGSLTSGTKL

SEQ ID NO: 75 - protein, 25 aa, Mus musculus
KTYNTNAQVPDSAGTATAYLCGVKA

SEQ ID NO: 76 - protein, 22 aa, Mus musculus
RTEENVSDGSPNAGTVEQTPKK

SEQ ID NO: 77 - protein, 12 aa, Mus musculus
RVSNGSPSLERM

SEQ ID NO: 78 - protein, 15 aa, Mus musculus
RSIQFVDWCPTGFKV

SEQ ID NO: 79 - protein, 32 aa, Mus musculus
RETDVPVDTNLIEFDTNYATDDDIVFEDFARL

SEQ ID NO: 80 - protein, 23 aa, Mus musculus
RSLDDLESATVSDGELTGADLKD

SEQ ID NO: 81 - protein, 25 aa, Mus musculus
RLKGQEDSLASAVDATTGQEACDSD

SEQ ID NO: 82 - protein, 18 aa, Mus musculus
RFDQLFDDESDPFEVLKA

SEQ ID NO: 83 - protein, 34 aa, Mus musculus
KGGEDEDGSDEDVVHNEDIHFEPIVSLPEVEVKS

SEQ ID NO: 84 - protein, 16 aa, Mus musculus
RDASPPPAFKPEPPKA

SEQ ID NO: 85 - protein, 34 aa, Mus musculus
KTPTPLMANDSTETSEAGEEEEDHEGDSENKERM

SEQ ID NO: 86 - protein, 18 aa, Mus musculus
RAQSTDSLGTSSSLQSKA

SEQ ID NO: 87 - protein, 27 aa, Mus musculus
ETEALLQSIGISPEPPLVPTPMSPSSK

SEQ ID NO: 88 - protein, 32 aa, Mus musculus
HLSNVSSTGSIDMVDSPQLATLADEVSASLAK

SEQ ID NO: 89 - protein, 16 aa, Mus musculus
SAESLQSLNSGLCPEK

SEQ ID NO: 90 - protein, 18 aa, Mus musculus
DLYQMSDSQLYEAFTFLK

SEQ ID NO: 91 - protein, 34 aa, Mus musculus
QSQGTTPDTTPARTPTEEGTPTSEQNPFLFQEGK

SEQ ID NO: 92 - protein, 32 aa, Mus musculus
QQESDDSLVDYGEGGEGQFNEDGSFIGQYTVK

SEQ ID NO: 93 - protein, 10 aa, Mus musculus
ASPVYLDILG

SEQ ID NO: 94 - protein, 21 aa, Mus musculus
DGSPDAPATPEKEEVAFSEYK

SEQ ID NO: 95 - protein, 21 aa, Mus musculus
LAAGAESPQPASGNSPSEDDR

SEQ ID NO: 96 - protein, 36 aa, Mus musculus
TLYEEAEEASDISQQVANLAISPTTPGPSWPGDALR

SEQ ID NO: 97 - protein, 34 aa, Mus musculus
GVNFAEEPMRSDSENGEEEEAAEAGAFNAPVINR

SEQ ID NO: 98 - protein, 27 aa, Mus musculus
EGTQASEGYFSQSQEEEFAQSEEPCAK

SEQ ID NO: 99 - protein, 21 aa, Mus musculus
ASGQAFELILKPPSPISEAPR

SEQ ID NO: 100 - protein, 22 aa, Mus musculus
LDDGHLNNSLGSPVQADVYFPR

SEQ ID NO: 101 - protein, 15 aa, Mus musculus
NLASPEGTLATLGLK

SEQ ID NO: 102 - protein, 32 aa, Mus musculus
GVVESVVTIEDDFITVVQTTTDEGESGSHSVR

SEQ ID NO: 103 - protein, 18 aa, Mus musculus
SQGSPEDPPSQASPGSNK

SEQ ID NO: 104 - protein, 39 aa, Mus musculus
SQQQKPEEEAVSSSQSPTATEIPGPLGSGLMPPLPFFNK

SEQ ID NO: 105 - protein, 27 aa, Mus musculus
VYNDGEQIIAQGDLADSFFIVESGEVK

SEQ ID NO: 106 - protein, 38 aa, Mus musculus
ASQPSPPAQEAGYSTLAQSYTPDHPSELPEEPSSPQER

SEQ ID NO: 107 - protein, 15 aa, Mus musculus
SKESVPDFPLSPPKK

SEQ ID NO: 108 - protein, 14 aa, Mus musculus
NFLNALTSPIEYQR

SEQ ID NO: 109 - protein, 37 aa, Mus musculus
EQPGNTVSSGQEDFPSVLFETAASLPSLSPLSTVSFK

SEQ ID NO: 110 - protein, 25 aa, Mus musculus
RPVSVSPSSSQEISENQYAVICSEK

SEQ ID NO: 111 - protein, 18 aa, Mus musculus
MDSYEQEEDIDQIVAEVK

SEQ ID NO: 112 - protein, 27 aa, Mus musculus
DGEPIENEEEDDEKHIFSDDSSELTIR

SEQ ID NO: 113 - protein, 26 aa, Mus musculus
ALYLEPSDGVSPTQETGEAQSQDDER

SEQ ID NO: 114 - protein, 36 aa, Mus musculus
APPASKASPAPTPTPAGAASPLAAVAAPATDAPQAK

SEQ ID NO: 115 - protein, 35 aa, Mus musculus
KEGGGDSSASSPTEEEQEQGEMSACSDEGTAQEGK

SEQ ID NO: 116 - protein, 14 aa, Mus musculus
AQSGSPESSSGQTK

SEQ ID NO: 117 - protein, 18 aa, Mus musculus
YLQDDTSDPTYTSSLGGK

SEQ ID NO: 118 - protein, 18 aa, Mus musculus
SISEGHPWHVPDSPSHSK

SEQ ID NO: 119 - protein, 8 aa, Mus musculus
RWTDLLSK

SEQ ID NO: 120 - protein, 29 aa, Mus musculus
QTPPASPSPQPIEDRPPSSPIYEDAAPFK

SEQ ID NO: 121 - protein, 20 aa, Mus musculus
DQGSCGSCWAFGAVEAISDR

SEQ ID NO: 122 - protein, 20 aa, Mus musculus
TVDLQDAEEAVELVQYAYFK

SEQ ID NO: 123 - protein, 16 aa, Mus musculus
AVESTDEDHQTDLDAK

SEQ ID NO: 124 - protein, 23 aa, Mus musculus
AADEDWDSELEDDLLGEDLLSGK

SEQ ID NO: 125 - protein, 23 aa, Mus musculus
KASEDESDLEDEEEKSQEDTEQK

SEQ ID NO: 126 - protein, 19 aa, Mus musculus
TPLATIADQQQLQLSPLKR

SEQ ID NO: 127 - protein, 24 aa, Mus musculus
LVGSGFPLPTSDLDDSLTEDIDEK

SEQ ID NO: 128 - protein, 11 aa, Mus musculus
IGEGTYGVVYK

SEQ ID NO: 129 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 130 - DNA, 7743 bp, Homo sapiens
acacagagcagctcctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaagagaaaaaaatcctgttcattttagtgttcaatttttttattattattgttgttcttatttaaccttgtaaaatatctataaatacaaaccaatttcattgtattctcactttgagggagatccagggggtgggaggggttgtggggagggggaaagcggagcactagaacatacaatctctctcccacgacaatctttttttattaaaagtctgctgttgtatactttaaaaacaggactcctgcctcatgccccttccacaaaagaagaaaacctttttctgtgctgatgggtttttttgaactttgttttcttttaaagtctagtgtgagactttggtatagtgcacagcttgaaattggttgggagcttagcaggtataactcaacggggacttaaatgtcacttgtaaaattaatccatatcttcgggtatttatagacttgcctttggcatgttggtggcaggtgtggcagacaaagaaatgtgtatcattcgtaacccagggaggtcaataaagtttggaactctacagggaagattcttagtagatttgttaaggttttgttttgctctcagttagtgctagtgatgtagaggcttgtacaggaggctgccagaggggaagcagcaagcaagactcaggcacacatgctctacaggtggctctttgtttgcctgaccaaagttctttgcaaatcttagcacagtttcaaactagtgacctgggaggagatggaaggggtgttgagcaggctgagctagctgctgaggtcaaaggctgatgagcccagaggaaggggacaggtcagggatacatctcaccactgtgaataagtttgtccagatttttttctaaagttacttcccttggaaagatacacttgagaggacattgtagttaaataatgtgaactgtaacagtcatctactggtttatttttcatattttttaattgaaaattgagcttgcagaaatagccacattctacacatagttctaattttaaatccaaatctagaatctgtatttaatttgttttttaacctcatgctttttacatttatttattgatgcatgtcagatggtagaaatattaaaaactacacatcagaatgatacagtcacttatacctgctgactttataggaaagctgatgatataaatgtgtgtatatatgttatatatacatatattcaatactgcctttttttttgtctacagtatcaaaattgactggttgaagcatgagaagaatgtttcccccacacccagttaagagtttttgtgtctgttttctttgtgtatcagtgaacgatgttaagaatcagtctctctttttgaagaaaaagcaatattccttggaaagcaaggagaattgaaggactatgtttgccgtgaggaaatagattttcatgactagtttgttttatacttttaaggttggcatctatgtgggccttatatactctaaaatgaactttagtcaccttggtgcttatgggccattacttgacctatgaatctttaaggcacaatcagttgtactttacatttaaagatcacttgagtgatggccgcctttccctcctacccgctccttccccacatgccttccaaggttagctggtaactgtagggctgcagagctgagcccatggttgtgtgtaacttgccctcaccctcctcattgccaccttaggtcactttatgggtctcgtcctccagagggttcggaagtggagtctgttggcagccctcctgcaggccctagcaccctgtcctgctccttaactgtgtgtgtgactctccaagagagttgtcctgcctgctgaagtgaaccagtacccagaaagacaactgtgagccatcttggttttcactcgctgtttagctgaggtcttgggccacaaaaggggtttcacaaacctctggatatatcagagtttatgagaaaggaaacatgctcagtcaaaccaaatcaaacaaattgaattttatgttttataaagtgcttctgaaagctaagatttgaaagaagtctgaaatcaaagtatttggcagcataactccttaaaggtagtggcgttgatagaccattttcagacagaatttataaagaatctgaaaaggcaggtctgtgatagagaaatggacctgcattcagatccaactgcccagcaagcgtttggatgcagacactgctctggacgtggtatactccccagagtccataaaaatcagtgcttattttaggaaacaggttgccccccacaactggggtaaaagaagagagaaaagtcacgcttttctctcatttcattgtgtgtgcatgtgtgcgtgtgtgtgtgtgtgtgtgtgtgctgagatgtgtgatttttctttctcaaggatcatggtgggatcacagaactcttttatacaagtgagatccaggtctctgaatatctttttgtatataataataataaaaagctcctcaccaaattcaagcttgtacattatattttctttctgtgtttttaaatttaagttttattgttttgtatgtaaatatgtggacccaggaactgttattaatgagcaaaaagttactgttcagggcagtgattctgtttaataatcagacaaaatgtagacgagctttttaaagccatatagttttaactctgtacagtaggtaccggcctgtattattgtaacaataactctagcaatgtatagtgtatctatatagtttggagtgccttcgcttccatgtgtttttttttttaatttgttcttttttaaattttaattggtttcctttatccatgtctccctgtccaccccctttccctttgaaataataactcactcataacagtatctttgccccttccacagttaagtttcagtgataccatactcaggagtgggaagaggaaatcatattcgtaatttcatttcgttgaagccctgcctttgttttggttctgaatgtctttcctcctcggtagcagtgagaccggtttcatttcatacttagtccattcagggacttagtgtagcaccagggagccctagagctggaggatatcgaatagattaaattttgctcgtctcttccacaagccctaaccatgggtcttaaaaacagcagattctgggagccttccatgctctctctctctcctcttttatctacttccctcccaaatgagagagtgacagagaattgtttttttataaatcgaagtttcttaatagtatcaggttttgatacgtcagtggtctaaaatgctatagtgcaattactagcagttactgcacggagtgccaccgtgccaatagaggactgttgttttaacaagggaactcttagcccatttcctccctcccgccatctctacccttgctcaatgaaatatcattttaatttcttttaaaaaaaatcagtttaattcttactgtgtgcccaacacgaaggccttttttgaaagaaaaatagaatgttttgcctcaaagtagtccatataaaatgtcttgaatagaagaaaaaactaccaaaccaaaggttactatttttgaaacatcgtgtgttcattccagcaaggcagaagactgcaccttctttccagtgacatgctgtgtcattttttttaagtcctcttaatttttagacacatttttggtttatgttttaacaatgtatgcctaaccagtcatcttgtctgcaccaatgcaaaggtttctgagaggagtattctctatccctgtggatatgaagacactggcatttcatctatttttccctttcctttttaaaggatttaactttggaatcttccaaaggaagtttggccaatgccagatccccaggaatttggggggttttctttcttttcaactgaaattgtatctgattcctactgttcatgttagtgatcatctaatcacagagccaaacacttttctcccctgtgtggaaaagtaggtatgctttacaataaaatctgtcttttctggtagaaacctgagccactgaaaataaaagagacaactagaagcacagtagagtcccagactgagatctacctttgagaggctttgaaagtaatccctggggtttggattattttcacaagggttatgccgttttattcaagtttgttgctccgttttgcacctctgcaataaaagcaaaatgacaaccagtacataaggggttagcttgacaaagtagacttccttgtgttaatttttaagtttttttttccttaactatatctgtctacaggcagatacagatagttgtatgaaaatctgcttgcctgtaaaatttgcatttataaatgtgttgccgatggatcacttgggcctgtacacataccaattagcgtgaccacttccatcttaaaaacaaacctaaaaaacaaaatttattatatatatatatatatatatataaaggactgtgggttgtatacaaactattgcaaacacttgtgcaaatctgtcttgatataaaggaaaagcaaaatctgtataacattattactacttgaatgcctctgtgactgatttttttttcattttaaatataaacttttttgtgaaaagtatgctcaatgttttttttccctttccccattcccttgtaaatacattttgttctatgtgacttggtttggaaatagttaactggtactgtaatttgcattaaataaaaagtaggttagcctggaaatgaaattaaaattcacaagtgtgtggtctttatttcagtacccaaccctcttccttcaccctactattttgccgctgcaatatgtagtcacatcaccatttccattcctctaattagggaaacattaatctttgttatacagaacaagatatcaataccacttcttgttctttccaatgattttattccattgtgtagccccaagaggtgcagcttccaccttggaaacctttggatttgatgtagaggaagctttgcagacactgcttagaaaagaaagaaaacaactctgaaagggacagtttttaaatgtgtataagctgctgtctttgattactgtgttcatgatttggtgtggctgtattttcttttaactttcatcctattagtaatggtctttgggggtctctgtaaaatatatggacaccacgaacagtggggctgtacctcccaggtaaccaacacatgttgtgtttgagtctgctcatttccaatactggatgatgtatgtaaacatgttatgtctcttagtgcaaaaagaaacatcatttttttagggctggctcactctgtcaggcctaatctaaaggctagatataaggtcatgtgactgctgcttcaataaaaacaaatttatattcga

SEQ ID NO: 131 - protein, 403 aa, Homo sapiens [sequence omitted]

SEQ ID NO: 132 - DNA, 8515 bp, Homo sapiens
gttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggctggcacatccagggacccgggccggttttaaacctcccgtgcgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcattgtgtgggtgttttattaaatgagagtttataattcaaattgcttaagtccattgaagttttaattaatgggcagccaaatgtgaatacaaagttttcagtttttttttttcctgctgtccttcaaagcctactgtttaaaaaaaaaaaaaaaaaaaaacatggcctgagagtagagtatctgtctactcatgtttaattaaggaaaaacacttatttttagggctttagtcatcacttcataaattgtataagcacattaaatagcgttctagtcctgaaaaagtccaagattcttagaaaattgtgcatatttttattatgacagatgtttgaagataattccccagaatggatttgatactttagatttcaattttgtggcttttgtctattattctgtactctgccatcagcatatggaaagcttcatttactcatcatgacttgtgccatataaaaattgatatttcggaatagtctaaaggactttttgtacttgaatttaatcatgttgtttctaatattcttaaaagcttgaagactaaagcatatcctttcaacaaagcatagtaaggtaataagaaagtgtagtttgtacaagtgttaaaaaaataaagtagacaatgttacagtgggacttattatttcaagtttacattttctccatgtaattttttaaaaagtaaatgaaaaaatgtgcaataatgtaaaatatgaagtgtatgtgtacacacattttatttttcggtatcttgggtatacgtatggttgaaaactatactggagtctaaaagtattctaatttataagaagacattttggtgatgtttgaaaaatagaaatgtgctagttttgtttttatatcatgtcctttgtacgttgtaatatgagctggcttggttcagtaaatgccatcaccatttccattgagaatttaaaactcaccagtgtttaatatgcaggcttccaaaggcttatgaaaaaaatcaagacccttaaatctagttaatttgctgctaacatgaaactctttggttcttttatttttgccagataattagacacacatctaaagcttagtcttaaatggcttaagtgtagctattgattagtgctgttgctagttcagaaagaaatgtttgtgaatggaaacaagaatattcagtccaaactgttgtaaggacagtacctgaaaaccaggaaacaggataatggaaaaagtcttttaaagatgaaatgttggagccaactttcttatagaattaattgtatgtggctatagaaagcctaatgattgttgcttatttttgagagcatattattcttttatgaccataatcttgctgtttttccatcttccaaaagatcttccttctaatatgtatatcagaatgtgggtagccagtcagacaaattcatattggttggtagctttaaaaagtttgtaatgtgaagacaggaaaggacaaaatagtttgctttggtggtagtactctggttgttaagctaggtattttgagactacttccccatcacaacaacaataaaataatcactcataatcctatcacctggagacatagccatcgttaatatgttagtgactatacaatcatgttttcttctgtatatccatgtatattctttaaaaatgaaatttatactgtacctgatctcaaagctttttagcttagtatatctgtcatgaatttgtaggatgttccattgcatcagaaaacggacagtgatttgattactttctaatgccacagatgcagattacatgtagttattgagaatcctttcgaattcagtggcttaatcatgaatgtctaaatattgttgacattaggatgatacatgtaaattaaagttacatttgtttagcatagacaagcttaacattgtagatgtttctcttcaaaaatcatcttaaacatttgcatttggaattgtgttaaatagaatgtgtgaaacactgtattagtaaacttcatcacctttctacttccttatagtttgaacttttcagtttttgtagttcccaaacagttgctcaatttagagcaaattaatttaacacctgccaaaaaaaggctgctgttggcttatcagttgtctttaaattcaaatgctcatgtgacttttatcacatcaaaaaatatttcattaatgattcacctttagctctgaaaattaccgcgtttagtaattatagtgggcttataaaaacatgcaactctttttgatagttatttgagaattttggtgaaaaatatttagctgagggcagtatagaacttataaaccaatatattgatatttttaaaacatttttacatataagtaaactgccatctttgagcataactacatttaaaaataaagctgcatatttttaaatcaagtgtttaacaagaatttatattttttattttttaaaattaaaaataatttatatttcctctgttgcatgaggattctcatctgtgcttataatggttagagattttatttgtgtggaatgaagtgaggcttgtagtcatggttctagtgtttcagtttgccaagtctgtttactgcagtgaaattcatcaaatgtttcagtgtggttttctgtagcctatcatttactggctatttttttatgtacacctttaggattttctgcctactctatccagttgtccaaatgatatcctacattttacaaatgccctttcagtttctattttctttttccattaaattgccctcatgtcctaatgtgcagtttgtaagtgtgtgtgtgtgtgtctgtgtgtgtgtgaatttgattttcaagagtgctagacttccaatttgagagattaaataatttaattcaggcaaacatttttcattggaatttcacagttcattgtaatgaaaatgttaatcctggatgacctttgacatacagtaatgaatcttggatattaatgaatttgttagtagcatcttgatgtgtgttttaatgagttattttcaaagttgtgcattaaaccaaagttggcatactggaagtgtttatatcaagttccatttggctactgatggacaaaaaatagaaatgccttcctatggagagtatttttcctttaaaaaattaaaaaggttaattattttgacta

SEQ ID NO: 133 - protein, 525 aa, Homo sapiens
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL

SEQ ID NO: 134 - DNA, 5428 bp, Homo sapiens
gcactgagcctaagcagccggtgatggcggcagcggctgtggtggctgcggcgggtccgggcccatgaggcgacgaaggaggcgggacggcttttacccagccccggacttccgagacagggaagctgaggacatggcaggagtgtttgacatagacctggaccagccagaggacgcgggctctgaggatgagctggaggaggggggtcagttaaatgaaagcatggaccatgggggagttggaccatatgaacttggcatggaacattgtgagaaatttgaaatctcagaaactagtgtgaacagagggccagaaaaaatcagaccagaatgttttgagctacttcgggtacttggtaaagggggctatggaaaggtttttcaagtacgaaaagtaacaggagcaaatactgggaaaatatttgccatgaaggtgcttaaaaaggcaatgatagtaagaaatgctaaagatacagctcatacaaaagcagaacggaatattctggaggaagtaaagcatcccttcatcgtggatttaatttatgcctttcagactggtggaaaactctacctcatccttgagtatctcagtggaggagaactatttatgcagttagaaagagagggaatatttatggaagacactgcctgcttttacttggcagaaatctccatggctttggggcatttacatcaaaaggggatcatctacagagacctgaagccggagaatatcatgcttaatcaccaaggtcatgtgaaactaacagactttggactatgcaaagaatctattcatgatggaacagtcacacacacattttgtggaacaatagaatacatggcccctgaaatcttgatgagaagtggccacaatcgtgctgtggattggtggagtttgggagcattaatgtatgacatgctgactggagcacccccattcactggggagaatagaaagaaaacaattgacaaaatcctcaaatgtaaactcaatttgcctccctacctcacacaagaagccagagatctgcttaaaaagctgctgaaaagaaatgctgcttctcgtctgggagctggtcctggggacgctggagaagttcaagctcatccattctttagacacattaactgggaagaacttctggctcgaaaggtggagcccccctttaaacctctgttgcaatctgaagaggatgtaagtcagtttgattccaagtttacacgtcagacacctgtcgacagcccagatgactcaactctcagtgaaagtgccaatcaggtctttctgggttttacatatgtggctccatctgtacttgaaagtgtgaaagaaaagttttcctttgaaccaaaaatccgatcacctcgaagatttattggcagcccacgaacacctgtcagcccagtcaaattttctcctggggatttctggggaagaggtgcttcggccagcacagcaaatcctcagacacctgtggaatacccaatggaaacaagtggcatagagcagatggatgtgacaatgagtggggaagcatcggcaccacttccaatacgacagccgaactctgggccatacaaaaaacaagcttttcccatgatctccaaacggccagagcacctgcgtatgaatctatgacagagcaatgcttttaatgaatttaaggcaaaaaaggtggagagggagatgtgtgagcatcctgcaaggtgaaacgactcaaaatgacagtttcagagagtcaatgtcattacatagaacacttcagacacaggaaaaataaacgtggattttaaaaaatcaatcaatggtgcaaaaaaaaacttaaagcaaaatagtattgctgaactcttaggcacatcaattaattgattcctcgcgacatcttctcaaccttatcaaggattttcatgttgatgactcgaaactgacagtattaagggtaggatgttgcttctgaatcactgttgagttctgattgtgttgaagaagggttatcctttcattaggcaaagtacaaaattgcctataatacttgcaactaaggacaaattagcatgcaagcttggtcaaactttttccagcaaaatggaagcaaagacaaaagaaacttaccaattgatgttttacgtgcaaacaacctgaatcttttttttatataaatatatatttttcaaatagatttttgattcagctcattatgaaaaacatcccaaactttaaaatgcgaaattattggttggtgtgaagaaagccagacaacttctgtttcttctcttggtgaaataataaaatgcaaatgaatcattgttaaccacagctgtggctcgtttgagggattggggtggacctggggtttattttcagtaacccagctgcaatacctgtctgtaatatgagaaaaaaaaaatgaatctatttaatcatttctacttgcagtactgctatgtgctaagcttaactggaagccttggaatgggcataagttgtatgtcctacatttcatcattgtcccgggcctgcattgcactggaaaaaaaaatcgccacctgttcttacaccagtatttggttcaagacaccaaatgtcttcagcccatggctgaagaacaacagaagagagtcaggataaaaaatacatactgtggtcggcaaggtgagggagatagggatatccaggggaagagggtgttgctgtggcccactctctgtctaatctctttacagcaaattggtaagattttcagttttacttctttctactgtttctgctgtctaccttccttatatttttttcctcaacagttttaaaaagaaaaaaaggtctatttttttttctcctatacttgggctacattttttgattgtaaaaatatttgatggccttttgatgaatgtcttccacagtaaagaaaacttagtggcttaatttaggaaacatgttaacaggacactatgtttttgaaattgtaacaaaatctacataaatgatttacaggttaaaagaataaaaataaaggtaactttacctttcttaaatatttcctgccttaaagagagcatttccatgactttagctggtgaaagggtttaatatctgcagagctttataaaaatatatttcagtgcatactggtataatagatgatcatgcagttgcagttgagttgtatcaccttttttgtttgtcttttataatgtcttcagtctgagtgtgcaaagtcaatttgtaatattttgcaaccctaggatttttttaaatagatgctgcttgctatgttttcaaacctttttgagccataggatccaagccataaaattctttatgcatgttgaattcagtcagaaaagagcaaggctttgctttttgaaattgcaactcaaatgagatgggatgaaatcctatgacagtaagcaaaaacagaaccatgaaaaatgattggacatacaccttttcaattgtggcaataattgaaagaatcgataaaagttcatctttggacagaaagcctttaaaaaaaaaatcactccctcttccccctcctcccttattgcagcagcctactgagaactttgactgttgctggtaaattagaagctacaataataattaagggcagaaattatacttaaaaagtgcagatccttgttctttgacaatttgtgatgtctgaaaaaacagaacccgaaaagctatggtgatatgtacaggcattatttcagactgtaaatggcttgtgatactcttgatacttgttttcaaatatgtttactaactgtagtgttgactgcctgaccaaattccagtgaaacttatacaccaaaatattcttcctaggtcctatttgctagtaacatgagcactgtgattggctggctataaccaccccagttaaaccattttcataattagtagtgccagcaatagtggcaaacactgcaacttttctgcataaaaagcattaattgcacagctaccatccacacaaatacatagtttttctgacttcacatttattaagtgaaatttatttcccatgctgtggaaagtttattgagaacttgtttcataaatggatatccctactatgactgtgaaaacatgtcaagtgtcacattagtgtcacagacagaaagcacacacctatgcaatatggcttatctatatttatttgtaaaaatccaagcatagtttaaaatatgatgtcgatattactagtcttgagtttctaagagggttctttatgttataccaggtaagtgtataaaagagattaagtgcttttttttcatcacttgattattttctttaaaatcagctattacaggatatttttttattttatacatgctgttttttaattaaaatataatcactgaagtttactaatttgattttataaggtttgtagcattacagaataactaaactgggatttataaaccagctgtgattaacaatgtaaagtattaattattgaactttgaaccagatttttaggaaaattatgttctttttccccctttatggtcttaactaatttgaatccttcaagaaggatttttccatactattttttaagatagaagataatttgtgggcaggggtggaggatgcatgtatgatactccataaattcaacattctttactataggtaatgaatgattataaacaagatgcatcttagatagtattaatatactgagccttggattatatatttaatataggacctattttgaatattcagttaatcatatggttcctagcttacaagggctagatctaagattattcccatgagaaatgttgaatttatgaagaatagattttaaggctttgaaaatggttaatttctcaaaaacatcaatgtccaaacatctaccttttttcataggagtagacactagcaagctggacaaactatcacaaaagtatttgtcacacataacctgtggtctgttgctgattaatacagtactttttcttgtgtgattcttaacattatagcacaagtattatctcagtggattatccggaataacatctgaaagatgggttcatctatgtttgtgtttgctctttaaactattgtttctcctatcccaagttcgctttgcatctatcagtaaataaaattcttcagctgccttattaggagtgctatgagggtaacacctgttctgcttttcatcttgtatttagttgactgtattatttgatttcggattgaatgaatgtaaatagaaattaaatgcaaatttgaatgaacataaatagaagtgatttatttttttattatcttaaagataggaaggaattagtatacgtatcaggtgtataaatggtttaaatttattttaacatgtcagatgtgttcaagttgtcatataaataaactatgtatgtaaggaatgggttaaaa